(19) United States
(12) Patent Application Publication
(10) Pub. No.: US 2022/0411482 A1
(43) Pub. Date: Dec. 29, 2022

(54) STABLE FORMULATIONS OF SILK-DERIVED PROTEIN

(71) Applicant: SILK TECHNOLOGIES, LTD., Plymouth, MN (US)

(72) Inventors: Brian D. Lawrence, Tampa, FL (US); David W. Infanger, Maple Grove, MN (US); Yue Bai, Plymouth, MN (US); Nicholas Paulson, Roseville, MN (US)

(73) Assignee: SILK TECHNOLOGIES, LTD., Plymouth, MN (US)

(21) Appl. No.: 17/785,392

(22) PCT Filed: Nov. 16, 2020
(86) PCT No.: PCT/US2020/060781

§ 371 Date: Jun. 14, 2022

Related U.S. Application Data
(60) Provisional application No. 62/936,294, filed on Nov. 15, 2019.
(60) Provisional application No. 63/094,709, filed on Oct. 21, 2020.
(60) Provisional application No. 63/094,748, filed on Oct. 21, 2020.

(51) Int. Cl. C07K 14/78; A61K 47/26; A61K 47/18; A61P 27/02

(52) U.S. Cl. CPC C07K 14/78; A61K 47/26; A61K 47/183; A61P 27/02

(57) ABSTRACT

A biotherapeutic ophthalmic solution that may include a silk-derived protein as an active ingredient. Ophthalmic formulations are critical to the delivery of dosage forms, user requirements, and maintaining product stability. The formulations described herein are ophthalmic solutions that are comfortable to the user while product stability is maintained, even after long-term storage. Numerous excipients, manufacturing processes, and container closures were evaluated for their ability to stabilize silk-derived protein under ambient and accelerated conditions. Analyses showed that a small sub-set of protein-containing formulations meet the high physiochemical property standards required for therapeutic ophthalmic solutions.

### RELATED APPLICATIONS

This application claims priority under 35 U.S.C. § 119(e) to U.S. Provisional Patent Application Nos. 63/094,748 filed Oct. 21, 2020, 63/094,709 filed Oct. 21, 2020, and 62/936,294 filed Nov. 15, 2019, which applications are incorporated herein by reference.

### GOVERNMENT SUPPORT

This invention was made with government support under Grant No. W81WH-17-C-0147 awarded by the United States Army. The government has certain rights in the invention.

### BACKGROUND OF THE INVENTION

Peptide and protein therapeutics are increasingly popular for the treatment of multiple diseases. Historic approaches to isolate these molecules included harvesting from animal organs and tissues. However, recent success in recombinant DNA technology has fueled the development of new protein biotherapeutics over the past two decades. The use of peptides, proteins, and protein-based biosimilars offer multiple advantages over chemically synthesized therapeutics for the treatment of disease. For example, purified antibodies, whose secondary and tertiary folding patterns underlie their structure, are remarkably target-specific and maintain functionality following introduction into the patient. Similarly, therapeutic peptides used to stimulate or inhibit cellular signaling (e.g., hormones, blood clotting factors) are potent and fast acting, and are metabolized using common protein degradative pathways of the host.

The efficacy of peptides and proteins relies on their ability to uniquely and effectively interface with their target, such as a cell surface receptor, lipid raft, or intracellular/extracellular molecule. This specificity requires the therapeutic peptide or protein to maintain a functional organization of amino acids and amino acid conformations that form into higher order secondary (e.g., alpha helical, beta sheet), tertiary (3-dimensional shape), or quaternary (multiple protein subunits interacting) structures. These arrangements are directed by electrostatic interactions between amino acid residues, including covalent (e.g., disulfide bonds) and non-covalent bonding (e.g., hydrogen bonding, hydrophobic bonds, ionic interactions), all of which rely on surrounding environmental parameters governed by physiologic homeostasis to promote these associations.

Slight changes in tissue or solution pH alter the concentration of hydrogen ions which in turn will promote or inhibit protonation of amino acid residues, thereby attracting or repelling neighboring amino acids with opposing or like charges, respectively. Similarly, increased temperature of a protein-containing tissue or solution elevates the internal energy, which can lead to protein instability due to peptide hydrolysis or protein structure rearrangement. Accordingly, there is a critical need for a controlled and maintained environment for peptide and protein drug formulations to maintain their efficacy.

While there are a multitude of processes and mechanisms that exist in the human body to maintain homeostasis—from ion channels and proton pumps at the cellular level, to the collective function of every organ in the body, to the systemic vasculature and lymphatic system for the eradication of fluid waste gradients in these organs and tissues—the removal of proteins from these feedback-driven safeguards render them vulnerable to impaired or lost functionality. This is especially true of therapeutic peptides and proteins, where ambient storage temperatures and non-physiologic solution conditions can rapidly degrade their functional structure. Changes to native protein structure can be due to the formation or cleavage break of covalent bonds, termed chemical instability, as well as the result of protein interactions with neighboring proteins or solution additives which impair protein solubility.

Chemical instability of proteins is commonly caused by oxidation (e.g., due to UV light exposure, and/or presence of peroxides or metal ions) or from amino acid deamidation that is instigated by changes in pH or elevated temperature. These latter changes in solution conditions can lead to protein flocculation and decreased protein solubility, which can arise from mechanical (e.g., shear) and interfacial stresses imposed on dissolved proteins in an aqueous solution. As such, the design of a protein-containing formulation must address as many of these stressors as possible to promote a shelf-stable therapeutic and maintain efficacy.

Currently, the primary strategy for therapeutic peptide and protein stability is to formulate a solution that mimics the physiologic environment of tissues. Salt-based buffer systems are commonly used to prevent large swings in solution pH that arise over time (e.g., with the absorption of carbon dioxide that acidifies the solution) or with peptide hydrolysis. Excipients are employed to increase solution osmolality and to reduce the opportunity for protein-protein interactions or flocculation. Similarly, the addition of surfactants is used to reduce interfacial stress and the potential for physical instability. Alternatively, many protein solutions are stored at refrigerated temperatures to extend shelf life, which is not ideal if the therapeutic is to be administered routinely or multiple times in a day. Lastly, some protein therapeutics are stored as lyophilized powders to minimize protein degradation. These solutions are solubilized immediately prior to administration but are prone to drug dosage errors due to variations in solvent volumes used for solubilization.

Accordingly, there is a need for a stable therapeutic protein formulation that are highly soluble in solution, have long term stability and low particulate count, and are compatible with other readily available components. There also is a need for protein formulations for treatment of eye-related conditions that can maintain the stability of a protein in solution for extended periods of time, thus increasing shelf-life and efficacy of the formulation. There is also a need for formulations that may be used to treat an eye-related condition without a protein additive. The present disclosure satisfies these needs.

### SUMMARY OF THE INVENTION

The invention provides a formulation for the physical and chemical stability of proteins such as modified silk fibroin. The silk-derived protein (SDP) described herein is a protein composition that has reduced beta-sheet activity, resulting in a highly soluble material. SDP can be readily incorporated into solution-based product formulations at high concentrations. Another advantage is that SDP has a high level of miscibility with other dissolved ingredients, such as those typically included in a therapeutic formulation.

Conventional agents used in pursuit of aqueous protein stability had no impact or negatively influenced the physical stability of SDP in solution, whereas the selection of the specific components of formulation described herein is unique. The specific buffering salts, osmotic agents, and surfactants extend the stability of SDP at room temperature without protein degradation or reduced protein efficacy.

This disclosure provides a formulation comprising a fibroin-derived protein composition wherein the average molecular weight of the fibroin-derived protein composition is 15-35 kDa. The formulation also comprises a buffering agent, polysorbate-80, and one or more osmotic agents; wherein the formulation has a pH of 4.5 to 6.0 and a particulate count of 50/mL or less after a storage period of greater than 12 weeks, or greater than 24 weeks, at 4° C. to 40° C., with respect to particulates having a diameter of 10 micrometers or more.

Additionally, this disclosure provides a formulation comprising about 0.1 wt. % to about 3 wt. % Silk Derived Protein-4 (SDP-4); polysorbate-80, about 10 millimolar to about 50 millimolar acetate buffer, and an osmotic agent; wherein the formulation has a pH of 5.2 to 5.8, an osmolality of 175 mOsm/kg to 185 mOsm/kg, and a particulate count of 50/mL or less after a storage period of greater than 12 weeks, or greater than 24 weeks, at 40° C., with respect to particulates having a diameter of 10 micrometers or more.

Certain embodiments include a formulation comprising about 0.1 wt. % to about 3 wt. % silk-derived protein wherein. The silk-derived protein can have a primary amino acid sequences of the fibroin-derived protein differ from native fibroin by at least 6% with respect to the absolute values of the combined differences in amino acid content of serine, glycine, and alanine; cysteine disulfide bonds between the fibroin heavy and fibroin light protein chains of the fibroin-derived protein are reduced or eliminated; the fibroin-derived protein comprises greater than 46% glycine amino acids and greater than 30% alanine amino acids; the fibroin-derived protein has a serine content that is reduced by greater than 40% compared to native fibroin protein such that the fibroin-derived protein comprises less than 8% serine amino acids; and the average molecular weight of the fibroin-derived protein is about 15 kDa to about 35 kDa; and polysorbate-80, about 10 millimolar to about 50 millimolar acetate buffer, and an osmotic agent; wherein the formulation has a pH of 5.2 to 5.8, an osmolality of 175 mOsm/kg to 185 mOsm/kg, and a particulate count of 50/mL or less after a storage period of greater than 12 weeks at 4° C. to 40° C. with respect to particulates having a diameter of 10 micrometers or more.

In one embodiment, the buffering salts produce a solution pH of 5.5; the buffering salts used have a functional range between 3.7 and 5.6; the osmotic agents used produce a solution with osmolality of 160-200 mOsm/kg; the osmolytes used have a concentration of 0.5% wt./wt. and 0.9% wt./wt; and the surfactant used has a concentration of 0.05-0.5% wt./wt.

In other aspects, certain embodiments provide an ophthalmologic formulation that may be used treat certain eye related conditions, and in particular, to treat or otherwise lessen the symptoms of dry eye disease.

Thus, preferred embodiments include ophthalmic formulations that may comprise about 0.04 wt. % to about 0.1 wt. % polysorbate-80, an acetate buffer comprising about 0.2 wt. % to about 0.3 wt. % sodium acetate and about 0.01 wt. % to about 0.03 wt. % acetic acid, and an osmotic agent comprising about 0.6 wt. % to about 0.9 wt. % dextrose and about 0.4 wt. % to about 0.9 wt. % magnesium chloride, wherein the formulation has a pH of 5.2 to 5.8 and an osmolality of 175 mOsm/kg to 185 mOsm/kg, and optionally may include a silk-derived protein.

One embodiment of an ophthalmic formulation consists essentially of about 0.04 wt. % to about 0.1 wt. % polysorbate-80, an acetate buffer comprising about 0.2 wt. % to about 0.3 wt. % sodium acetate and about 0.01 wt. % to about 0.03 wt. % acetic acid, and an osmotic agent comprising about 0.6 wt. % to about 0.9 wt. % dextrose and about 0.6 wt. % to about 0.9 wt. % magnesium chloride, wherein the formulation has a pH of 5.2 to 5.8 and an osmolality of 175 mOsm/kg to 185 mOsm/kg, and optionally may include a silk-derived protein.

In some embodiments, an ophthalmic formulation described herein further comprises a therapeutic protein or peptide composition. In certain embodiments, the wt. % of protein or peptide in the formulation is about 0.01% to about 15%. In certain embodiments, the wt % of protein or peptide is about 0.1% to about 5%, or about 1% to about 3%. In preferred embodiments, the protein is a hydrophobic protein. In one specific embodiment, the protein is SDP-4. In various embodiments, the wt. % of SDP-4 in the formulation is about 0.01% to about 15%. In additional embodiments, the wt % of SDP-4 is about 0.1% to about 5%, or about 1% to about 3%, or about 0.1%, 1%, or 3%.

Accordingly, certain embodiments of an ophthalmic formulation comprise one or more buffering agents, a surfactant, and one or more osmotic agents; wherein the formulation has a pH of 4.5 to 6.0 and the formulation maintains a protein in solution for a period greater than 4 weeks without gelation, and is capable of maintaining a particulate count of 50/mL or less after a storage period of greater than 12 weeks at 4° C. to 40° C., with respect to particulates having a diameter of 10 micrometers or more.

In additional embodiments, the ophthalmic formulation may comprise one or more surfactants; one or more osmotic agents; and an acetate buffering system comprising about 0.1 wt. % to about 1.0 wt. % sodium acetate and about 0.01 wt. % to about 0.1 wt. % acetic acid, wherein the buffering system maintains the formulation at a pH of 4.5 to 6.0, and the formulation is capable of maintaining a protein in solution for a period greater than 4 weeks without gelation, and the formulation is capable of maintaining a particulate count of 50/mL or less after a storage period of greater than 12 weeks at 4° C. to 40° C. with respect to particulates having a diameter of 10 micrometers or more, when protein is added to the ophthalmic formulation.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Temperature influence of the physical stability of SDP-4. Summary graph of solution particulate counts in 1.0% wt./wt. SDP-4 solutions maintained at defined temperature and pH (for 30 minutes). No buffering agents or excipients were used. The figure represents subvisible particulate counts using the Coulter method after 4 weeks in the indicated solution conditions. Particulate formation increased with increasing pH when solutions were maintained at 40 degrees Celsius.

FIG. 2. Temperature influence on the physical stability of SDP-4. Summary graph of solution particulate counts in 1.0% wt./wt. SDP-4 solutions maintained at defined temperature and pH (for 200 minutes). No buffering agents or excipients were used. The figure represents subvisible particulate counts using the Coulter method after 4 weeks in the indicated solution conditions. Particulate counts were remarkably decreased under all conditions with increased SDP-4 reaction time. Particulate formation increased with increasing pH when solutions were maintained at 40 degrees Celsius.

FIG. 3. Reaction time of SDP-4 influences physical stability. Summary graph of particulate counts (per Coulter method) in 1% wt./wt. SDP-4 solutions reacted at 30 (dark gray) or 200 (light gray) minutes and then buffered with citric acid (CA) to indicated solution pH. Solutions were stored at 40° C./75% Relative Humidity for 2 weeks prior to measurement. For all pH conditions, 30-minute reacted SDP-4 increased particulate counts relative to 200-minute reacted SDP-4.

FIG. 4. Assessment of the thermal stability of buffered SDP-4 solutions. Summary graph of particulate counts (per Coulter method) in 1% wt./wt. SDP-4 solutions reacted at 200 minutes and then buffered with citric acid to a pH of 5.5. Solutions were stored under defined temperature conditions for 2 weeks. Particulate formation was enhanced with increasing storage temperature.

FIG. 5. Container closure dramatically impacts the physical stability of SDP-4. Summary of particulate counts (per Coulter method) in 1.0% wt./wt. SDP-4 (200 min reaction) solutions stored in glass, low density polyethylene or polypropylene. No buffering agents or excipients were used. Solutions were stored at 40° C./75% relative humidity for 2 weeks prior to measurement. Particulate counts were lowest with a glass container and highest with a polypropylene container; low density polyethylene exhibited an intermediate particulate count.

FIG. 6. Buffering agent impact on the physical stability of SDP-4. Summary graph depicting particulate counts (per Coulter method) in 1% wt./wt. SDP-4 solutions (240 min reaction) buffered with glutamine, acetate, or histidine at concentrations of 10 or 50 mM, pH of 5.5. Solutions were stored in glass serum vials at 40° C./75% relative humidity for 8 weeks before measurements. Glutamate and acetate buffers inhibited particulate formation to a greater extent than histidine buffered solutions.

FIG. 7. Impact of buffer and buffer strength on pH drift. Summary graph of solution pH in formulations containing 1% wt./wt. SDP-4 (240 min reaction) solution buffered with glutamine, acetate, or histidine buffers at concentrations of 10 or 50 mM, pH of 5.5. Initial pH measurements were taken (dark, left side bar) and then again after 8 weeks (light, right side bar) at 40° C./75% relative humidity. Glutamine buffer failed to maintain pH of the SDP-4 solution, while 50 mM acetate and histidine buffers were effective at maintaining a stable pH.

FIG. 8. Impact of osmolality on SDP-4 stability. Summary graph depicting duration of solution stability until failure, defined by particulate counts of 50 or more particles of 10 to 25 μm in size. Two formulations containing 1.0% wt./wt. SDP-4 solution (240 min reaction), 25 mM acetate buffer (pH 5.5) and mannitol as the osmotic agent were stored in glass vials at 40° C./75% relative humidity. Higher osmolality (290 mOsm/kg) failed after one day; however, solutions with less mannitol (180 mOsm/kg) passed up to 14 days.

FIG. 9. Influence of osmotic agents on SDP-4 stability. Summary figure depicting physical stability (measured by particulate count, Coulter method) of 1% wt./wt. SDP-4 (240 min reaction) formulations buffered with acetate (25 mM) at pH 5.5 and defined salt or sugar osmotic agents (to achieve 180 mOsm/kg). Solutions were stored in glass vials at 40° C./75% relative humidity for 2 weeks. Mannitol and sodium chloride (NaCl) increased solution particulates, whereas MgCl₂ and dextrose reduced particulate formation. Regardless of composition, all solutions produced more 10-25 μm particulates relative to larger particulates measured.

FIG. 10. Polysorbate-80 enhances long term stability of SDP-4. Summary graph depicting duration of solution stability until failure, defined by particulate counts >50 ranging from 10 to 25 μm in size. Formulations containing 1% wt./wt. SDP-4 solution (240 min reaction), 25 mM acetate buffer (pH 5.5) and mannitol (to 180 mOsm/kg) were stored in glass vials at 40° C./75% relative humidity. The addition of polysorbate-80 extended the duration to failure from 1 day (for the control, no polysorbate-80) to 90 days.

FIG. 11. Impact of surfactant selection on the physical stability of SDP-4. Summary graph depicting particulate counts in 1% wt./wt. SDP-4 (240 min reaction) formulations containing 25 mM acetate buffer (pH 5.5), 38 mM magnesium chloride, and 39 mM dextrose with either 0.1% wt./wt. polysorbate-20 or polysorbate-80. Formulations were stored in glass vials under environmental conditions of 40° C./75% relative humidity for 4 weeks, then assessed for particulates by the Coulter method. The polysorbate-80 containing formulation had remarkably lower particulate counts than formulations containing polysorbate-20.

FIG. 12. A questionnaire given to patients in a clinical trial using certain embodiments of the formulations disclosed herein.

FIG. 13. Clinical trial design flow chart for ALPHA and BETA phase 2 clinical trials for the treatment of Dry Eye Disease.

FIG. 14. Primary clinical sign endpoint for Dry Eye study is termed Tear Break-Up Time (TBUT). (A) Standardized test procedure schematically shown. (B) SDP-4 (Amlisimod) increased TBUT compared to vehicle out to day 56 of the study (*p<0.001 for SDP-4 (n=75) vs. Vehicle (n=76), Error bars=SE).

FIG. 15. Treated group symptoms significantly improved in subpopulation Dry Eye subjects. (A) SANDE symptom scores improved most with SDP-4 (Amlisimod) eye drops on average at day 56 (p<0.1). (B) An identified patient subpopulation that did not include patients with starting SANDE scores greater than or equal to 70 showed a significant improvement over the whole population, demonstrating that SDP-4 (Amlisimod) is highly effective against vehicle in this patient population (*p=0.02, Vehicle n=32/76, 1% SDP-4 (Amlisimod) n=38/76; Error bars=SE). Y-axis=SANDE change from baseline (pts.) and X-axis=treatment days.

### DETAILED DESCRIPTION OF THE INVENTION

The invention provides ophthalmic formulations containing protein compositions derived from SDP. The protein compositions described herein include or can be prepared from the protein compositions described in U.S. Pat. No. 9,394,355 (Lawrence et al.), which is hereby incorporated by reference. Lower average molecular weight fractions can also be isolated to provide compositions with enhanced anti-inflammatory activity such as the protein compositions described in U.S. Patent Publication No. 2019/0169243 (Lawrence et al.), which is hereby incorporated by reference.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a component” includes a plurality of such components, so that a component X includes a plurality of components X. It is further noted that the claims may be drafted to exclude an optional element. As such, this statement is intended to serve as antecedent basis for the use of exclusive terminology, such as “solely,” “only,” “other than”, and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, element, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, an invention encompasses not only the entire group listed as a whole, but each member of the group individually and all possible subgroups of the main group. Additionally, for all purposes, an invention encompasses not only the main group, but also the main group absent one or more of the group members. An invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

For a therapeutic application, an “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is within the capacity of persons skilled in the art. The term “effective amount” is intended to include an amount of a composition described herein, or an amount of a combination of peptides described herein, e.g., that is effective to treat or prevent a disease or disorder, or to treat the symptoms of the disease or disorder, in a host. Thus, an “effective amount” generally means an amount that provides the desired effect.

Fibroin is a protein derived from the silkworm cocoon (e.g., Bombyx mori). Fibroin includes a heavy chain that is about 350-400 kDa in molecular weight and a light chain that is about 24-27 kDa in molecular weight, wherein the heavy and light chains are linked together by a disulfide bond. The primary sequences of the heavy and light chains are known in the art. The fibroin protein chains possess hydrophilic N and C terminal domains, and alternating blocks of hydrophobic/hydrophilic amino acid sequences allowing for a mixture of steric and electrostatic interactions with surrounding molecules in solution. At low concentration dilutions (1% or less) the fibroin protein molecule is known to take on an extended protein chain form and not immediately aggregate in solution. The fibroin protein is highly miscible with hydrating molecules such as hyaluronic acid (HA), polyethylene glycol (PEG), glycerin, and carboxymethyl cellulose (CMC), has been found to be highly biocompatible, and integrates or degrades naturally within the body through enzymatic action. Native fibroin (also referred to herein as prior art silk fibroin (PASF)), is known in the art and has been described by, for example, Daithankar et al. (Indian J. Biotechnol. 2005, 4, 115-121) and International Publication No. WO 2014/145002 (Kluge et al.).

The terms “silk-derived protein” (SDP) and “fibroin-derived protein” are used interchangeably herein. These materials are prepared by the processes described herein involving heat, pressure, and a high concentration of a heavy salt solution. Therefore ‘silk-derived’ and ‘fibroin-derived’ refer to the starting material of the process that structurally modifies the silk fibroin protein to arrive at a protein composition (SDP) with the structural, chemical and physical properties described herein. The SDP compositions possess enhanced solubility and stability in an aqueous solution. The SDP may be derived from silkworm silk (e.g., Bombyx mori), spider silk, or genetically engineered silk.

As used herein, the terms “molecular weight” and “average molecular weight” refer to weight average molecular weight determined by standard Sodium Dodecyl Sulfate Polyacrylamide Gel Electrophoresis (SDS-PAGE) electrophoresis methods undertaken with a NuPAGE™ 4%-12% Bis-Tris protein gel (ThermoFisher Scientific, Inc.) in combination analysis with ImageJ software (National Institutes of Health). ImageJ is used to determine the relative amount of protein of a given molecular weight in a sample. The software accomplishes this by translating the staining on the gel (i.e., the amount of protein) into a quantitative signal intensity. The user then compares this signal to a standard (or ladder) consisting of species of known molecular weights. The amount of signal between each marker on the ladder is divided by the whole signal. The cumulative summation of each protein sub-population, also referred to herein as fractions and interchangeably also referred to as fragments, allows the user to determine the median molecular weight, which is referred to herein as the average molecular weight. In practice, electrophoresis gels are stained, and then scanned into greyscale images, which are converted into histograms using ImageJ. Total pixel intensity within each gel lane is quantified by ImageJ (i.e., total area under the histogram), and subsequently fractionated into populations demarcated by protein molecular weight standards also stained on the gel. The histogram pixel area between any two molecular weight standards is divided by the total histogram area of the protein, thereby providing the fraction of total protein that falls within these molecular weights. Analysis by other methods may provide different values that account for certain peptides that are not accounted for by SDS-PAGE methods. For example, HPLC can be used to analyze the average molecular weights, which method provides values that are typically about 10-30% lower than determined by SDS-PAGE (increasing differences as molecular weights decrease).

### Embodiments of the Invention

This disclosure provides formulations comprising (a) a fibroin-derived protein composition wherein the primary amino acid sequences of the fibroin-derived protein composition differ from native fibroin by at least 4% with respect to the absolute values of the combined differences in amino acid content of serine, glycine, and alanine; cysteine disulfide bonds between the fibroin heavy and fibroin light protein chains of the fibroin-derived protein are reduced or eliminated; the protein composition has a serine content that is reduced by greater than 25% compared to native fibroin, wherein the serine content is at least about 5%; and the average molecular weight of the fibroin-derived protein composition is 15 to 35 kDa; and (b) a buffering agent, (c) polysorbate-80, and (d) one or more osmotic agents such that the mOsm is 170 mOsm/kg to about 300 mOsm/kg; wherein the formulation has a pH of 4.5 to 6.0 and a particulate count of 50/mL or less with respect to particulates having a diameter of 10 micrometers or more after a storage period of 12 weeks or more at 4° C. to 40° C.

In some embodiments, the protein composition comprises greater than 46.5% glycine amino acids, or the protein composition comprises greater than 30.5% alanine amino acids or greater than 31.5% alanine amino acids. In other embodiments, the protein composition has a serine content that is reduced by greater than 40% compared to native fibroin protein such that the protein composition comprises less than 8% serine amino acids. In additional embodiments, greater than 50% of the protein chains of the protein composition have a molecular weight within the range of 10 kDa to 40 kDa.

In further embodiments, the primary amino acid sequences of the fibroin-derived protein composition differ from native fibroin by at least by at least 6% with respect to the combined difference in serine, glycine, and alanine content; and the average molecular weight of the fibroin-derived protein is 12 to 30 kDa. In various other embodiments, the fibroin-derived protein composition is Silk Derived Protein-4 (SDP-4) having an average molecular weight of about 15 kDa to about 35 kDa, and the pH of the formulation is about 5.0 to about 6.0. In other embodiments, the pH is 5.2 to 5.8.

In various embodiments, the osmolality of the formulation is about 170 mOsm/kg to about 300 mOsm/kg. In some embodiments, the osmolality is about 160 mOsm/kg to about 200 mOsm/kg, about 175 mOsm/kg to about 180 mOsm/kg, about 180 mOsm/kg to about 200 mOsm/kg, about 200 mOsm/kg to about 250 mOsm/kg, or about 250 mOsm/kg to about 300 mOsm/kg.

The expression of weight percentage is to be interpreted as % wt./wt in this disclosure. In various embodiments, embodiments, the wt. % of SDP-4 in a formulation is about 0.01% to about 15%. In additional embodiments, the wt % of SDP-4 is about 0.1% to about 5%, or about 0.1%, about 1%, or about 3%. In some embodiments, the buffer comprises histidine, acetate, glutamate, or a combination thereof. In yet other embodiments, the formulation has a buffer concentration of about 10 millimolar to about 50 millimolar, or about 20 millimolar to about 40 millimolar. In other embodiments, the concentration of each of the one or more osmotic agents in the formulation is about 30 millimolar to about 40 millimolar, or about 35 millimolar. In other embodiments, the buffer comprises about 0.1 wt. % to about 1.0 wt. % sodium acetate and about 0.01 wt. % to about 0.1 wt. % acetic acid. In other embodiments, the buffer comprises about 0.5 wt. % to about 2.0 wt. % sodium acetate and about 0.05 wt. % to about 1.0 wt. % acetic acid.

In other embodiments, the osmotic reagent comprises a monosaccharide, an inorganic salt, or a combination thereof. In additional embodiments, the osmotic reagent comprises mannitol, dextrose, sodium chloride, magnesium chloride, or a combination thereof. In other embodiments, the osmotic reagent comprises about 0.1 wt. % to about 2 wt. % dextrose and about 0.1 wt. % to about 2 wt. % magnesium chloride. In yet other embodiments, the osmotic reagent comprises about 0.01 wt. % to about 2 wt. % dextrose and about 0.01 wt. % to about 2 wt. % magnesium chloride. In further embodiments, the wt. % of polysorbate-80 is about 0.02% to about 2%. In other embodiments, the wt. % of polysorbate-80 is about 0.01% to about 2%. In additional embodiments, the formulation is stored in a vessel comprising glass or polyethylene. In various embodiments, the vessel is a Type I borosilicate glass. In additional embodiments, the vessel can be a low-density polyethylene container. The formulation has been shown to be stable in low-density polyethylene container for greater than six months.

In other embodiments, the storage period or shelf-life is about 4 months to about 8 months, about 8 months to about 12 months, about 1 year to about 2 years, or more than 2 years from date of manufacture. In various embodiments, the particulate count after storage is about 200/mL, about 150/mL, about 100/mL, about 75/mL, about 45/mL, about 35/mL, about 25/mL, about 20/mL, about 15/mL, about 10/mL, about 5/mL or about 1/mL. In yet other embodiments, the storage temperature is about 10° C. to about 30° C., or 15° C. to about 25° C.

This disclosure also provides an aqueous formulation comprising about 0.1 wt. % to about 3 wt. % SDP-4 wherein the primary amino acid sequences of the SDP-4 differs from native fibroin by at least 6% with respect to the absolute values of the combined differences in amino acid content of serine, glycine, and alanine; cysteine disulfide bonds between the fibroin heavy and fibroin light protein chains of the SDP-4 are reduced or eliminated; the SDP-4 comprises greater than 46% glycine amino acids and greater than 30% alanine amino acids; the SDP-4 has a serine content that is reduced by greater than 40% compared to native fibroin protein such that the SDP-4 comprises less than 8% serine amino acids; and the average molecular weight of SDP-4 is about 15 kDa to about 35 kDa; and polysorbate-80, about 10 millimolar to about 50 millimolar acetate buffer, and an osmotic agent; wherein the formulation has a pH of 5.2 to 5.8, an osmolality of 175 mOsm/kg to 185 mOsm/kg, and a particulate count of 50/mL or less after a storage period of greater than 12 weeks at 4° C. to 40° C. with respect to particulates having a diameter of 10 micrometers or more.

In one preferred embodiment, a formulation may consist essentially of a fibroin-derived protein composition wherein the primary amino acid sequences of the fibroin-derived protein composition differ from native fibroin by at least 4% with respect to the absolute values of the combined differences in amino acid content of serine, glycine, and alanine, cysteine disulfide bonds between the fibroin heavy and fibroin light protein chains of the fibroin-derived protein are reduced or eliminated, the protein composition has a serine content that is reduced by greater than 25% compared to native fibroin, wherein the serine content is at least about 5%, wherein the average molecular weight of the fibroin-derived protein composition is less than 35 kDa and greater than 15 kDa, a buffering agent, polysorbate-80, and one or more osmotic agents, wherein the formulation has a pH of 4.5 to 6.0 and a particulate count of 50/mL or less after a storage period of greater than 12 weeks at 4° C. to 40° C. with respect to particulates having a diameter of 10 micrometers or more.

In another preferred embodiment, a formulation may consist essentially of about 0.1 wt. % to about 3 wt. % SDP-4 wherein the primary amino acid sequences of the fibroin-derived protein differs from native fibroin by at least 6% with respect to the absolute values of the combined differences in amino acid content of serine, glycine, and alanine, cysteine disulfide bonds between the fibroin heavy and fibroin light protein chains of the fibroin-derived protein are reduced or eliminated, the fibroin-derived protein comprises greater than 46% glycine amino acids and greater than 30% alanine amino acids, the fibroin-derived protein has a serine content that is reduced by greater than 40% compared to native fibroin protein such that the fibroin-derived protein comprises less than 8% serine amino acids, and the average molecular weight of the SDP-4 is about 15 kDa to about 35 kDa, and polysorbate-80, about 10 millimolar to about 50 millimolar acetate buffer, and an osmotic agent, wherein the formulation has a pH of 5.2 to 5.8, an osmolality of 175 mOsm/kg to 185 mOsm/kg, and a particulate count of 50/mL or less after a storage period of greater than 12 weeks at 4° C. to 40° C. with respect to particulates having a diameter of 10 micrometers or more.

In various embodiments, the acetate buffer comprises about 0.2 wt. % to about 0.3 wt. % sodium acetate and about 0.01 wt. % to about 0.03 wt. % acetic acid. In some embodiments, the osmotic agent comprises about 0.6 wt. % to about 0.9 wt. % dextrose and about 0.6 wt. % to about 0.9 wt. % magnesium chloride. In additional embodiments, the wt. % of polysorbate-80 is about 0.05% to about 0.1%.

In various embodiments, a formulation may comprise a fibroin-derived protein (e.g., SDP-4) as prepared herein present in a final concentration of about 0.1% w/w, sodium acetate present in a final concentration of about 0.25% w/w, glacial acetic acid in a final concentration of about 0.01% w/w, magnesium chloride present in a final concentration of about 0.8% w/w, dextrose present in a final concentration of about 0.8% w/w, and polysorbate-80 present in a final concentration of about 0.05% w/w.

In various embodiments, a formulation may comprise a fibroin-derived protein (e.g., SDP-4) as prepared herein present in a final concentration of about 1% w/w, sodium acetate present in a final concentration of about 0.25% w/w, glacial acetic acid in a final concentration of about 0.01% w/w, magnesium chloride present in a final concentration of about 0.75% w/w, dextrose present tin a final concentration of about 0.75% w/w, and polysorbate-80 present in a final concentration of about 0.05% w/w.

In some embodiments, a formulation may comprise a fibroin-derived protein (e.g., SDP-4) as prepared herein present in a final concentration of about 3% w/w, sodium acetate present in a final concentration of about 0.25% w/w, glacial acetic acid in a final concentration of about 0.01% w/w, magnesium chloride present in a final concentration of about 0.65% w/w, dextrose present in a final concentration of about 0.65% w/w, and polysorbate-80 present in a final concentration of about 0.05% w/w.

Additionally, this disclosure provides a method for treating an ophthalmic disease comprising administering an effective amount of the formulation disclosed above to a subject having an ophthalmic disease, thereby treating the ophthalmic disease. In some embodiments, the ophthalmic disease is dry eye syndrome.

The formulations described herein provide effective treatment and/or reduce the symptoms of eye related conditions. These results are surprising at least in part because the prevailing art discourages a person of ordinary skill in the art from selecting the particular combination of components used in the inventors' formulations. For example, Wang et al., Dual Effects of Tween 80 on Protein Stability., Int J Pharm. 2008 Jan. 22; 347(1-2):31-8, which is directed to studies of the effect of TWEEN-80 on stability and aggregation of the model protein IL-2, discloses that the “[a]ddition of 0.1% Tween 80 significantly increased the rate of IL-2 mutein aggregation during storage” (Wang, Abstract, page 31). However, the inventors found that the use of a polysorbate as a surfactant (e.g., polysorbate 80) in the formulation significantly inhibited aggregation of proteins in solution.

Further, the inventors' formulations unexpectedly display characteristics that contradict the prevailing art. Katakam et al., Effects of Surfactants on the Physical Stability of Recombinant Human Growth Hormone., J Pharm Sci. 1995 June; 84(6):713-6, is directed to the effects of certain surfactants (e.g., BRIJ 35, TWEEN-80, Pluronic F68) on the physical stability of human growth hormone upon exposure to air/water interfaces and non-isothermal stress. Katakam discloses that TWEEN-80 (i.e., polysorbate 80) did not protect hGH from thermal stress: “surfactants at concentration that stabilized hGH with respect to interfacial denaturation [from agitation] did not give any protection against thermal stress”. (Katakam, page 716, 2^{nd} full para.). In contrast, the inventors found that the use of TWEEN-80 (polysorbate 80) protected the protein in solution from thermal stress.

Kreilgaard et al., Effect of Tween 20 on Freeze-Thawing- and Agitation-induced Aggregation of Recombinant Human Factor XIII., J Pharm Sci. 1998 December; 87(12):1597-603 is directed to studying the studying the effects of polysorbate 20 (i.e., TWEEN-20) on freeze-thawing-induced aggregation of recombinant human factor XIII (rFXIII). Kreilgaard discloses that “[t]hese observations suggest that Tween 20 stabilizes rFXIII [protein] primarily by competing with stress-induced soluble aggregates for interfaces, inhibiting subsequent transition to insoluble aggregates” (Kreilgaard, page 1602, last full para.). Additionally, Bam et al., Tween Protects Recombinant Human Growth Hormone against Agitation Induced Damage via Hydrophobic Interactions., J Pharm Sci. 1998 December; 87(12):1554-9 discloses “[i]n the absence of surfactants, recombinant human growth hormone rapidly forms insoluble aggregates during agitation. The nonionic surfactant TWEEN-20, when present at surfactant:protein molar ratios >4, effectively inhibits this aggregation.” (Bam, Abstract). These studies present results that are directly opposite to those found by the inventors—that the use of polysorbate 20 (TWEEN-20) actually destabilized the protein in solution, leading to an increase in aggregation and formation of insoluble particulates.

Furthermore, the prevailing art teaches the use of osmolytes/polyols, such as glycerol, to prevent protein aggregation in solution. For example, Vagenende et al., Mechanisms of Protein Stabilization and Prevention of Protein Aggregation by Glycerol., Biochemistry 2009 Nov. 24; 48(46):11084-96 discloses that “glycerol prevents protein aggregation by inhibiting protein unfolding and by stabilizing aggregation-prone partially unfolded intermediates through preferential interactions with hydrophobic surface regions that favor amphiphilic interface orientations of glycerol” (Vagenende, page 11094, 4^{th} full para). Similarly, Feng et al., Effects of glycerol on the compaction and stability of the wild type and mutated rabbit muscle creatine kinase., Proteins 2008 May 1; 71(2):844-54 discloses that in the presence of glycerol in the refolding buffer, “the aggregation of both proteins behaved similarly: decreased as glycerol concentration increased, and was fully inhibited in 30% glycerol”. (Feng, page 850, first paragraph). (Also see Prieve et al., Glycerol decreases the Volume and Compressibility of Protein Interior., Biochemistry 1996, 35, 2061-2066 which states that “we propose that glycerol induces a release of the so-called ‘lubricant’ water, which maintains conformational flexibility by keeping apart neighboring segments of the polypeptide chain” (hence increasing protein stability) (Prieve, Abstract); Gekko et al., Mechanism of Protein Stabilization: Preferential Hydration in Glycerol- Water Mixtures., Biochemistry 1981, 20, 4667-4676 that teaches “[t]he present measurements of the preferential interactions of proteins with solvent components in the water-glycerol solvent system have shown that of six proteins examined, all are preferentially hydrated in this solvent system . . . . It would appear reasonable, therefore, to generalize this situation for other proteins. Furthermore, it has been known empirically for a long time that the conformation of proteins is stabilized by the presence of glycerol.” (Gekko, page 4674, 2^{nd} full para.); Sedgwick et al., Protein Phase Behavior and Crystallization: Effect of Glycerol., J. Chem. Phys. 2007 Sep. 28; 127(12):125102 that teaches “[w]e find that at a fixed protein concentration, and increasing amount of salt is needed for protein crystallization and crystallization takes progressively longer as the glycerol concentration is increased”. (Sedgwick, page 6, 3^{rd} full para)). Surprisingly, the inventors found that the use of glycerol in the formulations greatly accelerated protein aggregation and the formation of insoluble particulates.

Furthermore, Chen et al., Influence of Histidine on the Stability and Physical Properties of a Fully Human Antibody in Aqueous and Solid Forms., Pharm Res. 2003 December; 20(12):1952-60 discloses the utility of using histidine to prevent protein aggregation: “[i]ncreasing the histidine concentration in the bulk solution inhibited the increases of high-molecular-weight (BMW) species and aggregates upon lyophilization and storage. In addition, histidine bulk enhanced solution stability of the antibody under freezing and thermal stress conditions, as evidenced by the lower levels of aggregates.” (Chen, Abstract). Additionally, Shiraki et al., Amino Acid Esters Prevent Thermal Inactivation and Aggregation of Lysozyme, Biotechnol Prog. 2005 21: 640-643, teaches the advantageous use of amino acid esters in the prevention of thermal inactivation of proteins: “amino acid esters (AAEs) prevent heat induced aggregation and inactivation of hen egg lysozyme. Lysozyme was completely inactivated (<1% original activity) during heat treatment at 98° C. for 30 min in a solution containing 0.2 mg/mL lysozyme in 50 mM Na-phosphate buffer (pH 6.5)”. (Shiraki, Abstract). In contrast to these studies, the inventors found that low concentrations of amino acid esters (arginine) did not prevent protein aggregations while the use of high concentrations of amino acid esters led to gelation of the formulation. While the inventors found that histidine increased protein stability in solution, histidine was not suitable for use in ophthalmic formulations because of histidine-induced irritation caused to the eye to which the formulation was applied.

With respect to the use of buffers and ions with the formulations, the prevailing art teaches the advantages of using calcium ions to stabilize proteins in solution. For example, Saboury et al., Effects of calcium binding on the structure and stability of human growth hormone., Int J Biol Macromol. 2005 Sep. 28; 36(5):305-9 discloses “[c]alcium ions binding increase the protein thermal stability by increasing of the alpha helix content as well as decreasing of both beta and random coil structures”. (Sarboury, Abstract). Additionally, Pikal-Cleland et al., Effect of glycine on pH changes and protein stability during freeze-thawing in phosphate buffer systems., J Pharm Sci. 2002 September; 91(9):1969-79 teaches the advantages of using glycine to minimize discrete pH microenvironment formation during solution freezing which underlie protein instability: “[t]he presence of glycine at higher concentration (>100 mM) in the sodium phosphate buffer resulted in a more complete crystallization of the disodium salt as indicated by the frozen pH values closer to the equilibrium value (pH 3.6)”. (Pikal-Cleland, Abstract). However, the inventors found that the use of calcium ions negatively impacted protein solubility in solution while the use of glycine had no discernible impact on protein stability.

Given the teachings of the prevailing art, a person of ordinary skill in the art would not be motivated to pursue a formulation comprising the combination of components selected by Applicant, nor could the person of ordinary skill in the art produce the formulations and associated features with any reasonable expectation of success that the formulations would be effective in treating eye-related conditions, and, in particular, dry eye disease.

### Preparation of SDP Compositions

The protein compositions used in the ophthalmic formulations can be prepared as described in U.S. Pat. No. 9,394,355 (Lawrence et al.) and U.S. Patent Publication No. 2019/0169243 (Lawrence et al.). The SDP can be derived from Bombyx mori silkworm fibroin or other fibroin from the Bombyx genus or other silk proteins.

SDP material can be prepared by the following process.

1. Silk cocoons are prepared by removing pupae material and pre-rinsing in warm water.

2. Native fibroin protein fibers are extracted from the gum-like sericin proteins by washing the cocoons in water at high water temperature, typically 95° C. or more, at alkaline pH.

3. The extracted fibroin fibers are dried and then dissolved using a solvent system that neutralizes hydrogen bonding between the beta-sheets; a 54% LiBr aqueous solution of 20% w/v silk fibroin protein is effective for this neutralization step.

4. The fibroin protein dissolved in LiBr solution is processed in an autoclave environment (˜121° C. [˜250° F.], at ˜15-17 PSI pressure, for approximately 30 minutes at temperature).

5. The heat-processed fibroin protein and LiBr solution are then dialyzed to remove lithium and bromide ions from the solution. At this point in the process the material has been chemically transformed to SDP.

6. The dialyzed SDP is then filtered to remove any non-dissolved aggregates and contaminating bioburden.

The SDP solution is produced using a distinctly different process than the process used for current silk fibroin solution production. Notably, autoclaving the silk fibroin protein in LiBr solution (not after LiBr is removed) initiates chemical transitions that produce the stabilized SDP material. The fibroin protein is dissolved in the LiBr solution, neutralizing hydrogen bonding and electrostatic interactions of the solubilized native fibroin protein. Under these conditions, the protein lacks specific secondary structure confirmations in solution. As a result, the thermodynamic energy required to hydrolyze covalent bonding within the fibroin protein chain is at its lowest, thereby facilitating hydrolytic cleavage and confirmational changes after formation of dehydro-alanine structures.

SDP preparatory conditions include a temperature set to 121° C. for 30 minutes at 15-17 PSI in an autoclave. However, in various embodiments, the processing conditions may be modified to stabilize the SDP material to varying degrees. Additional protein solubilization agents can be used in the process, including other or additional halide salts such as calcium chloride and sodium thiocyanate, organic agents such as urea, guanidine hydrochloride, and 1,1,1,3,3,3-hexafluoroisopropanol, additional strong ionic liquid solution additives such as calcium nitrate and 1-butyl-3-methylimidazolium chloride, or a combination thereof.

### SDP Compositions

SDP composition described herein can be derived from silk fibroin and possess enhanced solubility and stability in aqueous solutions. The compositions can be used to treat and reduce inflammation. In one embodiment, the SDP and/or fractions thereof have primary amino acid sequences that differ from native fibroin by at least 4% (via summation of the absolute values of the differences) with respect to the combined amino acid content of serine, glycine, and alanine. In some embodiments, a plurality of the protein fragments of SDP can terminate in amide (—C(═O)NH₂) groups. SDP can have a serine content that is reduced by greater than 40% compared to native fibroin, wherein the serine content is at least about 5%. The cysteine disulfide bonds between the fibroin heavy and fibroin light protein chains of fibroin may be reduced or eliminated. In certain embodiments, at least 75 percent of the protein fragments have a molecular weight of less than about 60 kDa. The composition may comprise less than 8.5% serine amino acid residues. In some embodiments, the average molecular weight of the SDP is less than 55 kDa. The SDP compositions possess enhanced stability in an aqueous solution.

In some cases, the SDP protein compositions are prepared by a process comprising heating an aqueous fibroin solution at an elevated pressure. The aqueous fibroin solution includes lithium bromide at a concentration of at least 8M. The aqueous fibroin solution is heated to at least about 105° C. (221° F.) under a pressure of at least about 10 PSI for at least about 20 minutes, to provide the protein composition. As a result of these processing conditions, the polypeptides of the protein composition comprise less than 8.5% serine amino acid residues, and a plurality of the protein fragments terminate in amide (C(═O)NH₂) groups.

In some cases, SDP compositions are prepared by a process comprising heating an aqueous fibroin solution at an elevated pressure, wherein the aqueous fibroin solution comprises lithium bromide at a concentration of 9-10M, and wherein the aqueous fibroin solution is heated to a temperature in the range of about 115° C. (239° F.) to about 125° C. (257° F.), under a pressure of about 15 PSI to about 20 PSI for at least about 20 minutes; to provide the protein composition. The protein composition can include less than 6.5% serine amino acid residues.

In some cases, methods of preparing can use lithium bromide having a concentration between about 8.0M and about 11M. In some embodiments, the concentration of lithium bromide is about 9M to about 10M, or about 9.5M to about 10M.

In some embodiments, the aqueous fibroin solution that contains lithium bromide is heated to at least about 107° C. (225° F.), at least about 110° C. (230° F.), at least about 113° C. (235° F.), at least about 115° C. (239° F.), or at least about 120° C. (248° F.).

In some embodiments, the aqueous fibroin solution that contains lithium bromide is heated under a pressure of at least about 12 PSI, at least about 14 PSI, at least about 15 PSI, or at least about 16 PSI, up to about 18 PSI, or up to about 20 PSI.

In some embodiments, the aqueous fibroin solution that contains lithium bromide is heated for at least about 20 minutes, at least about 30 minutes, at least about 45 minutes, or at least about 1 hour, up to several (e.g., 12-24) hours.

SDP compositions are chemically distinct from native silk fibroin protein as a result of the preparation process, resulting in changes in amino acid content and the formation of terminal amide groups. The resulting SDP has enhanced solubility and stability in aqueous solution. The SDP can be used in a method for forming, for example, ophthalmic formulations with a protein composition described herein, for example, an aqueous solution of the protein composition. The solution can include about 0.01% to about 35% w/v SDP. The solution can be about 65% to about 99.9% w/v water.

In some embodiments, SDP is prepared using a process that induces hydrolysis, amino acid degradation, or a combination thereof, of fibroin protein such that the average molecular weight of the protein is reduced from about 100-200 kDa for silk fibroin produced using prior art methods to about 35-90 kDa, or about 40-50 kDa, for the SDP material described herein. The resulting polypeptides can be a random assortment of peptides of various molecular weights averaging to the ranges recited herein.

In addition, the amino acid chemistry can be altered by reducing cysteine content to levels non-detectable by standard assay procedures. For example, the serine content can be reduced by over 50% from the levels found in the native fibroin, which can result in increases of overall alanine and glycine content by 5% (relative amino acid content), as determined by standard assay procedures. The SDP material can have a serine content of less than about 8% relative amino acid content, or a serine amino acid content of less than about 6% relative amino acid content. The SDP material can have a glycine content above about 46.5%, and/or an alanine content above about 30% or above about 30.5%. The SDP material can be absent of detectable cysteine content, for example, as determined by HPLC analysis of the hydrolyzed polypeptide of the protein composition. The SDP material can form 90% less, 95% less, or 98% less beta-sheet secondary protein structures as compared to native silk fibroin protein, for example, as determined by the FTIR analysis.

SDP compositions possess enhanced stability in aqueous solution, wherein: the primary amino acid sequences of the SDP composition differs from native fibroin by at least 4% with respect to the combined (absolute value) difference in serine, glycine, and alanine content (SDP vs. PASF); cysteine disulfide bonds between the fibroin heavy and fibroin light protein chains are reduced or eliminated; and the composition has a serine content that is reduced by greater than 25% compared to native fibroin protein. The average molecular weight of the SDP composition can be less than 60 kDa and greater than about 35 kDa, or greater than about 40 kDa, as determined by the MWCO of the dialyzing membrane and SD S-PAGE analysis.

In some cases, SDP compositions possess primary amino acid sequences that differ from native fibroin by at least 6% with respect to the combined difference in serine, glycine, and alanine content; cysteine disulfide bonds between the fibroin heavy and fibroin light protein chains are reduced or eliminated; and the composition has a serine content that is reduced by greater than 40% compared to native fibroin protein. The average molecular weight of the SDP composition can be less than about 55 kDa and greater than about 35 kDa, as determined by the MWCO of the dialyzing membrane and SDS-PAGE analysis.

In some cases, SDP compositions possess primary amino acid sequences modified from native silk fibroin; cysteine disulfide bonds between the fibroin heavy and fibroin light protein chains are reduced or eliminated; the average molecular weight of the SDP composition is less than about 60 kDa and greater than about 35 kDa; and a 5% w/w aqueous solution of the SDP composition maintains an optical absorbance at 550 nm of less than 0.25 for at least two hours after five seconds of sonication.

In some cases, SDP compositions possess enhanced stability in aqueous solutions, wherein: the primary amino acid sequences of the SDP composition is modified from native silk fibroin such that they differ from native fibroin by at least 5% with respect to the combined (absolute value) difference in serine, glycine, and alanine content. In some embodiments, the difference of is at least 6%, 8%, 10%, 12% or 14% compared to native fibroin. Cysteine disulfide bonds between the fibroin heavy and fibroin light protein chains are reduced or eliminated; the average molecular weight of the SDP composition is less than about 60 kDa and greater than about 35 kDa; and the SDP composition maintains an optical absorbance at 550 nm of less than 0.2 for at least two hours after five seconds of sonication.

SDP compositions can be isolated and/or purified as a dry powder or film, for example, by dialysis and/or filtration. Alternatively, SDP compositions can be isolated and/or purified as stable aqueous solutions, which can be modified for use as a therapeutic formulation, such as an ophthalmic formulation described herein.

In various embodiments, the amino acid composition of the SDP can differ from the amino acid composition of native fibroin by at least 4%, by at least 4.5%, by at least 5%, or by at least 5.5%, or by at least 6%, with respect to the content of serine, glycine, and alanine combined.

In some cases, the SDP compositions described herein have a serine content that is reduced by greater than 25%, by greater than 30%, by greater than 35%, by greater than 40%, or by greater than 45%, compared to the serine content of native fibroin protein.

The average molecular weight of SDP compositions can be less than about 80 kDa, less than about 70 kDa, less than about 60 kDa, or less than about 55 kDa, or the composition has an average molecular weight of about 50-60 kDa, or about 51-55 kDa. In various embodiments, the average molecular weight of the SDP composition can be greater than 35 kDa, greater than about 40 kDa, or greater than about 50 kDa. Accordingly, the (weight average) average molecular weight of SDP compositions can be about 36 kDa to about 80 kDa, about 36 kDa to about 65 kDa, about 36 kDa to about 60 kDa, or about 40 kDa to about 55 kDa. In various embodiments, the average molecular weight of the SDP composition is about 45 kDa to about 65 kDa, about 45 kDa to about 60 kDa, about 50 kDa to about 65 kDa, or about 50 kDa to about 60 kDa.

The SDP compositions can be soluble in water at 40% w/w without any precipitation observable by ocular inspection.

In some embodiments, the SDP compositions comprise less than 8% serine amino acid residues. In some cases, protein compositions comprise less than 7.5% serine amino acid residues, less than 7% serine amino acid residues, less than 6.5% serine amino acid residues, or less than 6% serine amino acid residues. The serine content of the peptide compositions is generally at least about 4%, or at least about 5%, or about 4-5%.

In some embodiments, SDP compositions comprise greater than 46.5% glycine amino acids, relative to the total amino acid content of the protein composition. In some cases, protein compositions comprise greater than 47% glycine amino acids, greater than 47.5% glycine amino acids, or greater than 48% glycine amino acids.

In some embodiments, the SDP compositions comprise greater than 30% alanine amino acids, relative to the total amino acid content of the protein composition. In some cases, protein compositions comprise greater than 30.5% alanine, greater than 31% alanine, or greater than 31.5% alanine.

In some embodiments, the SDP compositions can completely re-dissolve after being dried to a thin film. In various embodiments, protein compositions can lack beta-sheet protein structure in aqueous solution. The protein composition can maintain an optical absorbance in aqueous solution of less than 0.25 at 550 nm after at least five seconds of sonication.

In some embodiments, the SDP protein compositions can be in combination with water. In some cases, protein compositions can completely dissolve in water at a concentration of 10% w/w, or even greater concentrations such as 15% w/w, 20% w/w, 25% w/w, 30% w/w, 35% w/w, or 40% w/w. In some embodiments, protein compositions can be isolated and purified, for example, by dialysis, filtration, or a combination thereof.

In various embodiments, the SDP compositions can enhance the spreading of an aqueous solution comprising the protein composition and ophthalmic formulation components, for example, compared to the spreading of a corresponding composition that does not include the protein composition. This enhanced spreading can result in an increase in surface area of the aqueous solution by greater than twofold, or greater than threefold.

In various embodiments, the SDP compositions do not form a gel at concentrations up to 20% w/v, up to 30% w/v, or up to 40% w/v in water. In some embodiments, SDP compositions can have glycine-alanine-glycine-alanine (GAGA) (SEQ ID NO: 1) segments of amino acids that comprise at least about 47.5% of the amino acids of the SDP composition. In some cases, SDP compositions can also have GAGA (SEQ ID NO: 1) segments of amino acids that comprise at least about 48%, at least about 48.5%, at least about 49%, at least about 49.5%, or at least about 50%, of the amino acids of the protein composition.

In various embodiments, the SDP compositions can have glycine-alanine (GA) segments of amino acids that comprise at least about 59% of the amino acids of the SDP composition. In some cases, SDP compositions can also have GA segments of amino acids that comprise at least about 59.5%, at least about 60%, at least about 60.5%, at least about 61%, or at least about 61.5%, of the amino acids of the protein composition. In typical embodiments, the fibroin has been separated from sericin. In various embodiments, the SDP composition re-dissolves after drying as a thin film, a property not found with native fibroin.

In one specific embodiment, the invention provides an SDP composition prepared by a process comprising heating an aqueous fibroin solution at an elevated pressure, wherein the aqueous fibroin solution comprises lithium bromide at a concentration of 9-10 M, and wherein the aqueous fibroin solution is heated to a temperature in the range of about 115° C. (239° F.) to about 125° C. (257° F.), under a pressure of about 15 PSI to about 20 PSI for at least about 30 minutes; to provide the protein composition, wherein the protein composition comprises less than 6.5% serine amino acid residues. The protein composition has an aqueous viscosity of less than 10 cP as a 15% w/w solution in water.

Stability Evaluations. The stability of a protein solution can be evaluated a number of different ways. One suitable evaluation is the Lawrence Stability Test (U.S. Pat. No. 9,394,355 (Lawrence et al.). Another suitable evaluation is the application of sonication to a protein solution, followed by optical absorbance analysis to confirm continued optical clarity (and lack of aggregation, beta-sheet formation, and/or gelation). Standard sonication, or alternatively ultrasonication (sound frequencies greater than 20 kHz), can be used to test the stability of an SDP solution. Solutions of SDP are stable after subjecting to sonication. The SDP composition maintains an optical absorbance at 550 nm of less than 0.25 for at least two hours after five seconds of sonication. For example, a 5% w/w solution of the protein composition maintains an optical absorbance of less than 0.1 at 550 nm after five seconds of sonication at ˜20 kHz, the standard conditions used for the sonication described herein. In various embodiments, SDP composition aqueous solutions do not gel upon sonication at concentrations of up to 10% w/w. In further embodiments, SDP composition aqueous solutions do not gel upon ultrasonication at concentrations of up to 15% w/w, up to 20% w/w, up to 25% w/w, up to 30% w/w, up to 35% w/w, or up to 40% w/w.

Low viscosity. As a result of its preparation process and the resulting changes in the chemical structures of its peptide chains, SDP has a lower viscosity than native silk fibroin (PASF). As a 5% w/w solution in water (at 25.6° C.), native silk fibroin has a viscosity of about 5.8 cP, whereas under the same conditions, SDP has a viscosity of about 1.8 cP, and SDP-4 has a viscosity of about 2.7 cP (e.g., 2.6-2.8 cPs). SDP maintains a low viscosity compared to PASF at higher concentrations as well. The SDP composition can have an aqueous viscosity of less than 5 cP, or less than 4 cP, as a 10% w/w solution in water. In various embodiments, SDP remains in solution up to a viscosity of at least 9.8 cP. SDP also has an aqueous viscosity of less than 10 cP as a 15% w/w solution in water. SDP can also have an aqueous viscosity of less than 10 cP as a 24% w/w solution in water.

The process described herein provides a protein composition where the fibroin light chain protein is not discernable after processing, as well when the sample is run using standard Sodium Dodecyl Sulfate Polyacrylamide Gel Electrophoresis (SDS-PAGE) electrophoresis methods undertaken with a NuPAGE™ 4%-12% Bis-Tris protein gel (ThermoFisher Scientific, Inc.). Furthermore, the resulting SDP material forms minimal to no beta-sheet protein secondary structure post-processing, while silk fibroin solution produced using prior art methods forms significant amounts of beta-sheet secondary structure. In one embodiment, the SDP material can be prepared by processing silk fibroin fibers under autoclave or autoclave-like conditions (i.e., approximately 120° C. and 14-18 PSI) in the presence of a 40-60% w/v lithium bromide (LiBr) solution

### SDP Composition Fractions

Silk Technologies, Ltd. has developed a silk-derived protein (SDP) product that can be readily incorporated into ophthalmic product formulations for reducing inflammation and enhancing the wound healing process. The SDP product can be separated into smaller protein fractions or sub-populations based on molecular weight to enhance the anti-inflammatory and wound healing properties. SDP protein sub-populations, also referred to as fractions or fragments, can be separated by any suitable and effective method, for example, by size exclusion chromatography or membrane dialysis. For example, the fractions can be separated in to 2-4 different groups based on decreasing average molecular weights. Example 6 describes one method for preparing four different fractions that have the same overall amino acid content and terminal amide content but different average molecular weights. It was surprisingly discovered that the different fractions also possess different biological properties, for example, for reducing inflammation in the body and in various tissues as a result of differences in cellular uptake of the different fractions.

Low average molecular weight fractions of SDP reduce inflammation and treat dry eye. Also described are compositions for treating ocular conditions, such as, but not limited to, dry eye disease, and/or injury, including corneal wounds. The treatments can include the administration of a formulation that includes SDP, or a low molecular weight SDP sub-population (SDP-4). In certain embodiments, the invention provides methods for treating a disease state and/or wound comprising administering to a subject in need thereof a composition comprising low molecular weight SDP (e.g., SDP-4).

SDP-4 is a subpopulation of SDP protein wherein the primary amino acid sequences that differ (via summation of absolute value differences) from native fibroin by at least 4% with respect to the combined amino acid content of serine, glycine, and alanine. A plurality of the protein fragments can terminate in amide (—C(═O)NH₂) groups. SDP-4 compositions have a serine content that is reduced by greater than 40% compared to native fibroin, wherein the serine content is at least about 5%. The cysteine disulfide bonds between the fibroin heavy and fibroin light protein chains of fibroin may be reduced or eliminated. In some embodiments, at least 75 percent of the protein fragments have a molecular weight of less than about 100 kDa. Such compositions reduce inflammation and promote cell migration and/or proliferation in the tissue to treat the disease state and/or enhance closure of the wound. The SDP compositions possess enhanced solubility and stability in an aqueous solution.

SDP composition fractions can have an average molecular weight between about 15 kDa and 60 kDa. In one embodiment, a low molecular weight fraction having an average molecular weight of about 15-35 kDa is isolated, which fraction is referred as SDP-4.

In some embodiments, at least 60 percent of the protein fragments have a molecular weight of less than about 60 kDa, or less than about 55 kDa, to promote cell migration and proliferation in the tissue to close the wound. In another embodiment, at least 90 percent of the protein fragments have a molecular weight of less than about 100 kDa and promote cell migration and proliferation in the tissue to close the wound.

In some embodiments, at least 80 percent of the protein fragments have a molecular weight between about 10 kDa and 85 kDa. In some embodiments, at least 50 percent of the protein fragments have a molecular weight between about 18 kDa and 60 kDa. In some embodiments, at least 85 percent of the protein fragments have a molecular weight of greater than about 12 kDa. In some embodiments, at least 90 percent of the protein fragments have a molecular weight of greater than about 10 kDa.

In certain embodiments, the invention provides an SDP composition comprising low molecular weight SDP and a pharmaceutically acceptable carrier. The low molecular weight SDP can have an average molecular weight of less than 60 kDa.

In one preferred embodiment, the SDP-4 fraction has an average molecular weight of 15-35 kDa, as determined by SDS-PAGE/ImageJ analysis, as previously described above, and a pH 8.1-8.3, an osmolarity of about 23 mOsm, and a viscosity of about 1.5-3 cP at 25° C., each as a 50 mg/mL solution in water.

In one preferred embodiment, the SDP-4 fraction has an average molecular weight of 15-30 kDa, as determined by SDS-PAGE/ImageJ analysis, as previously described above, and a pH 8.1-8.3, an osmolarity of about 23 mOsm, and a viscosity of about 1.5-3 cP at 25° C., each as a 50 mg/mL solution in water.

In one preferred embodiment, the SDP-4 fraction has an average molecular weight of 15-25 kDa, as determined by SDS-PAGE/ImageJ analysis, as previously described above, and a pH 8.1-8.3, an osmolarity of about 23 mOsm, and a viscosity of about 1.5-3 cP at 25° C., each as a 50 mg/mL solution in water.

In another preferred embodiment, the SDP-4 fraction has an average molecular weight of about 18-22 kDa, as determined by SDS-PAGE/ImageJ analysis, as previously described above, and a pH of about 8.1-8.3, an osmolarity of about 23 mOsm, and a viscosity of about 1.5-3 cP at 25° C., each as a 50 mg/mL solution in water.

In some SDP-4 fraction embodiments, about 39% of the protein fragments of SDP-4 are between the range of 25 kDa to 50 kDa, about 57.7% of the protein fragments are between the range of 20 kDa to 60 kDa, about 72.1% of the protein fragments are between the range of 15 kDa to 85 kDa, about 83.6% of the protein fragments are between the range of 10 kDa to 85 kDa, and about 85.3% of the protein fragments are between the range of 10 kDa to 100 kDa.

Various SDP compositions can be prepared to include low molecular weight protein fragments or high molecular weight protein fragments or combinations thereof. Low molecular weight protein fragments reduce inflammation and/or enhance cell migration and/or proliferation on a diseased tissue surface and/or wound. Low molecular weight protein fragments are also useful in treating inflamed tissue surfaces due to an active disease state and/or the presence of a wound or wounds. In some cases, it may be useful to apply a composition of low molecular weight protein fragments to enhance the wound healing process. These cases may include wounds acquired on the battlefield during war, surgical wounds of a person who desires faster healing, for example, of an infection or for pain relief. The wound healing process is enhanced by increasing cell numbers, reducing inflammatory molecules, such as MMP-9, and/or increasing epithelial cell proliferation.

High molecular weight protein fragments may increase cell adhesion to the basement membrane or aid in basement membrane formation. In some cases, it may be useful to apply a composition of high molecular weight protein fragments for chronic wounds or wounds that fester or wounds that have difficulty healing up, such as diabetic ulcers or skin burns. Whereas low molecular weight protein fragments may be involved in wound closure rate, high molecular weight protein fragments are involved in wound closure quality. In some cases, it may be used to apply a composition of carefully selected amounts of low molecular weight protein fragments and high molecular weight protein fragments for optimal wound healing rate and quality. The wound healing process is enhanced by increasing structural proteins, such focal adhesion kinases (FAK) and/or tight junctions between cells, such as zonula occluden (ZO-1) structures.

Low average molecular weight fractions such as SDP-4 possess certain properties making the fraction distinct from SDP and higher molecular weight fractions. For example, SDP cellular uptake is dependent on molecular weight of the peptide chains. SDP peptide molecules smaller than about 60 kDa in size are readily absorbed by cells in culture, and more specifically human corneal limbal epithelial (hCLE) cells. SDP molecules larger than about 60 kDa in size are mostly excluded from being absorbed by the cell cultures. It is also important to note that SDP molecules do not co-localize with lysosomal-associated membrane protein 1 (LAMP-1), which is a marker for the lysosomal endocytotic degradation pathway. As a result, the SDP molecules appear to associate with a non-specified cellular membrane receptor, in which molecules of less than about 60 kDa are then absorbed by the hCLE cells. More importantly, because the SDP molecules are not absorbed through the lysosomal degradation pathway, they are bioavailable and able to elicit biological activity.

### Aqueous SDP Formulations

The SDP compositions and sub-fractions described herein can be formulated with water and/or a pharmaceutical carrier. In a specific embodiment, the carrier is acetate buffered saline, for example, in an ocular formulation.

In some embodiments, ophthalmic compositions are provided for the treatment of dry eye syndrome in a human or mammal. Compositions provided herein can be an aqueous solution that includes an amount of SDP effective for treating dry eye syndrome. For example, the effective amount of the SDP in the aqueous solution can be about 0.01% by weight to about 80% by weight SDP. In other embodiments, the aqueous solution can include SDP at about 0.1% by weight to about 10% by weight, or about 0.5% by weight to about 2% by weight. In certain specific embodiments, the ophthalmic composition can include about 0.05% w/v SDP, about 0.1% w/v SDP, about 0.2% w/v SDP, about 0.25% w/v SDP, about 0.5% w/v SDP, about 0.75% w/v SDP, about 1% w/v SDP, about 1.5% w/v SDP, about 2% w/v SDP, about 2.5% w/v SDP, about 5% w/v SDP, about 8% w/v SDP, or about 10% w/v SDP.

In various embodiments, the ophthalmic formulation can include additional components in the aqueous solution, such as a demulcent agent, a buffering agent, and/or a stabilizing agent. The demulcent agent can be, for example, hyaluronic acid (HA), hydroxyethyl cellulose, hydroxypropyl methylcellulose, dextran, gelatin, a polyol, carboxymethyl cellulose (CMC), polyethylene glycol, propylene glycol (PG), hypromellose, glycerin, polysorbate 80, polyvinyl alcohol, or povidone. The demulcent agent can be present, for example, at about 0.01% by weight to about 10% by weight, or at about 0.2% by weight to about 2% by weight. In one specific embodiment, the demulcent agent is HA. In various embodiments, the HA can be present at about 0.2% by weight of the formulation. One or more of these components can also be excluded from the formulation.

The buffering or stabilizing agent of an ophthalmic formulation can be phosphate buffered saline, borate buffered saline, citrate buffer saline, sodium chloride, calcium chloride, magnesium chloride, potassium chloride, sodium bicarbonate, zinc chloride, hydrochloric acid, sodium hydroxide, edetate disodium, or a combination thereof. One or more of these components can also be excluded from the formulation.

An ophthalmic formulation can further include an effective amount of an antimicrobial preservative. The antimicrobial preservative can be, for example, sodium perborate, polyquaterium-1 (e.g., Polyquad® preservative), benzalkonium (BAK) chloride, sodium chlorite, brimonidine, brimonidine purite, polexitonium, or a combination thereof. One or more of these components can also be excluded from the formulation.

An ophthalmic formulation can also include an effective amount of a vasoconstrictor, an antihistamine, or a combination thereof. The vasoconstrictor or antihistamine can be naphazoline hydrochloride, ephedrine hydrochloride, phenylephrine hydrochloride, tetrahydrozoline hydrochloride, pheniramine maleate, or a combination thereof. One or more of these components can also be excluded from the formulation.

In one embodiment, an ophthalmic formulation can include an effective amount of SDP as described herein in combination with water and one or more ophthalmic components. The ophthalmic components can be, for example, a) polyvinyl alcohol; b) PEG and hyaluronic acid; c) PEG and propylene glycol, d) CMC and glycerin; e) propylene glycol and glycerin; f) glycerin, hypromellose, and PEG; or a combination of any one or more of the preceding components. The ophthalmic formulation can include one or more inactive ingredients such as HP-guar, borate, calcium chloride, magnesium chloride, potassium chloride, zinc chloride, and the like. The ophthalmic formulation can also include one or more ophthalmic preservatives such as sodium chlorite (Purite® preservative (NaClO₂), polyquad, BAK, EDTA, sorbic acid, benzyl alcohol, and the like.

Ophthalmic components, inactive ingredients, and preservatives can be included at about 0.1% to about 5% w/v, such as about 0.15%, 0.2%, 0.25%, 0.3%, 0.4%, 0.5%, 0.75%, 1%, 1.5%, 2%, 2.5%, or 5%, or a range in between any two of the aforementioned values.

SDP is highly stable in water, where shelf life solution stability is more than twice that of native silk fibroin in solution. For example, the SDP is highly stable in water, where shelf life solution stability is more than 10 times greater compared to native silk fibroin in solution. The SDP material, when in an aqueous solution, does not gel upon sonication of the solution at a 5% (50 mg/mL) concentration. In other embodiments, the SDP material, when in an aqueous solution, does not gel upon sonication of the solution at a 10% (100 mg/mL) concentration.

### Aqueous Opthalmic Formulations

The disclosure also generally provides certain ophthalmological and/or aqueous formulations that may, for example, be used to treat an eye relate condition. Applicant has found that the use of certain ingredients in an ophthalmologic formulation such as an acetate buffering system and low pH level (below neutral pH levels) are surprisingly effective in treating dry eye disease. Further, Applicant has found also that the formulations disclosed herein are effective in stabilizing a protein in solution for unexpectedly long periods of time while simultaneously showing low level of particulates. The use of a combination of specific buffering agents, osmotic agents, and surfactants was identified that, not only is surprisingly effective in treating dry eye disease, but also extends the use of certain proteins at room temperature without protein degradation or reduced protein efficacy.

Accordingly, exemplary formulations include one or more buffering agents, a surfactant, and one or more osmotic agents. In some embodiments, the formulations also may include a pH level of about 4.5 to 6.0. The formulations optionally may include a protein that may be stabilized in solution for extended periods of time. In these embodiments, the formulation is capable of, for example, maintaining the protein in solution for a period greater than 4 weeks without gelation, and is capable of maintaining a particulate count of 50 particles/mL or less after a storage period of greater than 12 weeks at 4° C. to 40° C., with respect to particulates having a diameter of 10 micrometers or more.

In some embodiments, the buffering agents may comprise histidine, acetate, glutamate, or a combination thereof. In yet other embodiments, the formulation includes one or more buffering agents having a final concentration of about 10 millimolar to about 50 millimolar, or about 20 millimolar to about 40 millimolar. In other embodiments, the concentration of each of the one or more osmotic agents in the formulation is about 30 millimolar to about 40 millimolar, or about 35 millimolar.

In preferred embodiments, the buffering agents may comprise about 0.1 wt. % to about 1.0 wt. % sodium acetate and about 0.01 wt. % to about 0.1 wt. % acetic acid. In other embodiments, the buffer comprises about 0.5 wt. % to about 2.0 wt. % sodium acetate and about 0.05 wt. % to about 1.0 wt. % acetic acid.

Preferably, the buffering agents (e.g., sodium acetate and glacial acetic acid) maintain a pH of the formulation of about 4.5 to about 6.0, about 5.0 to about 6.0, about 5.2 to about 5.8, about 5.3 to about 5.7, or about 5.4, or about 5.5.

In some embodiments, the osmotic agents in the formulation may comprise a monosaccharide, an inorganic salt, or a combination thereof. In additional embodiments, the osmotic agent may comprise mannitol, dextrose, sodium chloride, magnesium chloride, or a combination thereof. In other embodiments, the osmotic agent may comprise about 0.1 wt. % to about 2 wt. % dextrose and about 0.1 wt. % to about 2 wt. % magnesium chloride. In yet other embodiments, the osmotic agent may comprise about 0.01 wt. % to about 2 wt. % dextrose, and about 0.01 wt. % to about 2 wt. % magnesium chloride. In further embodiments, the osmotic agent may comprise about 0.6 wt. % to about 0.9 wt. % dextrose and about 0.6 wt. % to about 0.9 wt. % magnesium chloride.

Certain embodiments of a formulation also may include one or more surfactants. Surfactants include, but are not limited to, non-ionic detergents, that is, a detergent that includes molecules with head groups that are uncharged. Non-ionic detergents include polyoxyethylene (and related detergents), and glycosidic compounds (e.g., alkyl glycosides). Alkyl glucosides include octyl β-glucoside, n-dodecyl-β-D-maltoside, beta-decyl-maltoside, and Digitonin. Examples of polyoxyethylene detergents include polysorbates (e.g., Polysorbate 40, polysorbate 60, polysorbate 80 (also known as TWEEN-40, TWEEN-60, and TWEEN-80, respectively), TRITON-X series (e.g., TRITON X-100), TERGITOL series of detergents (e.g., NP-40), the BRIJ series of detergents (e.g., BRIJ-35, BRIJ-58, BRIJ-L23, BRIJ-L4, BRIJ-010), and PLURONIC F68. Preferably, the surfactant is polysorbate 40, polysorbate 60, or polysorbate 80. In certain preferred embodiments, the surfactant is polysorbate 80. Preferably, the surfactant is present in a formulation having a final concentration of about 0.02% to about 1% w/w, and more preferably, at a final concentration of about 0.02% to about 0.5% w/w. In one certain preferred embodiment, polysorbate 80 is present in a final concentration of about 0.01% to about 2.0% or about 0.02% to about 0.5%. In another embodiment, the only surfactant present in the formulation is polysorbate 80.

In various embodiments, the osmolality of the formulation is about 170 mOsm/kg to about 300 mOsm/kg. In other embodiments, the osmolality is about 160 mOsm/kg to about 200 mOsm/kg, about 175 mOsm/kg to about 180 mOsm/kg, about 180 mOsm/kg to about 200 mOsm/kg, about 200 mOsm/kg to about 250 mOsm/kg, or about 250 mOsm/kg to about 300 mOsm/kg. In one preferred embodiment, the osmolality is about 160 mOsm/kg to about 280 mOsm/kg or about 175 mOsm/kg to about 185 mOsm/kg.

In additional embodiments, the formulation is stored in a vessel comprising glass or polyethylene. In various embodiments, the vessel is a Type I borosilicate glass. In additional embodiments, the vessel can be a low-density polyethylene container. The formulation has been shown to be stable in low-density polyethylene container for greater than six months.

In other embodiments, the storage period or shelf-life of the formulation is about 4 months to about 8 months, about 8 months to about 12 months, about 1 year to about 2 years, or more than 2 years from date of manufacture. In various embodiments, the particulate count after storage is about 200/mL, about 150/mL, about 100/mL, about 75/mL, about 45/mL, about 35/mL, about 25/mL, about 20/mL, about 15/mL, about 10/mL, about 5/mL or about 1/mL. In yet other embodiments, the storage temperature is about 10° C. to about 30° C., or 15° C. to about 25° C.

One preferred embodiment of an ophthalmic or aqueous formulation comprises about 0.04 wt. % to about 0.1 wt. % polysorbate-80, an acetate buffer comprising about 0.2 wt. % to about 0.3 wt. % sodium acetate and about 0.01 wt. % to about 0.03 wt. % acetic acid, and an osmotic agent comprising about 0.6 wt. % to about 0.9 wt. % dextrose and about 0.6 wt. % to about 0.9 wt. % magnesium chloride such that the formulation has a pH of 5.2 to 5.8 and an osmolality of 175 mOsm/kg to 185 mOsm/kg.

One certain preferred embodiment of an ophthalmic or aqueous formulation comprises one or more surfactants, one or more osmotic agents, and an acetate buffering system comprising about 0.1 wt. % to about 1.0 wt. % sodium acetate and about 0.01 wt. % to about 0.1 wt. % acetic acid, wherein the buffering system maintains the formulation at a pH of 4.5 to 6.0.

One preferred embodiment of an ophthalmic or aqueous formulation comprises 0.1% to 1.0% sodium acetate, 0.01% to 0.1% acetic acid, 0.1% to 2% magnesium chloride, 0.1% to 2% dextrose, 0.02% to 2% polysorbate-80, an osmolality of about 160-200 mOsm/kg, and a pH of about 4.5-6.

One preferred embodiment of an ophthalmic or aqueous formulation comprises about 0.25 sodium acetate, about 0.01% acetic acid, about 0.75% magnesium chloride, about 0.75% dextrose, about 0.05% polysorbate-80, an osmolality of about 160-200 mOsm/kg, and a pH of about 4.5-6.

Another preferred embodiment of an ophthalmic or aqueous formulation comprises 0.1% to 1.0% sodium acetate, 0.01% to 0.1% acetic acid, 0.1% to 2% magnesium chloride, 0.1% to 2% dextrose, 0.02% to 2% polysorbate-80, an osmolality of about 160-200 mOsm/kg, and a pH of about 4.5-6.

Another preferred ophthalmic or aqueous formulation comprises about 0.25 sodium acetate, about 0.01% acetic acid, about 0.75% magnesium chloride, about 0.75% dextrose, about 0.05% polysorbate-80, and has an osmolality of about 180-190 mOsm/kg and a pH of 5.2-5.7.

Another preferred embodiment of an ophthalmic or aqueous formulation consists essential of 0.1% to 1.0% sodium acetate, 0.01% to 0.1% acetic acid, 0.1% to 2% magnesium chloride, 0.1% to 2% dextrose, 0.02% to 2% polysorbate-80, an osmolality of about 160-200 mOsm/kg, and a pH of about 4.5-6.

In yet a further preferred embodiment, an ophthalmic or aqueous formulation consists essentially of about 0.25 sodium acetate, about 0.01% acetic acid, about 0.75% magnesium chloride, about 0.75% dextrose, about 0.05% polysorbate-80, an osmolality of about 180-190 mOsm/kg, and a pH of 5.2-5.7.

In certain embodiments, the ophthalmic or aqueous formulation may stabilize a protein in solution for extended periods of time. For example, the formulations are capable of maintaining a protein in solution, if present, for a period greater than 4 weeks without gelation, and the formulation is capable of maintaining a particulate count of 50/mL or less after a storage period of greater than 12 weeks at 4° C. to 40° C. with respect to particulates having a diameter of 10 micrometers or more.

In various embodiments, the wt. % of protein in the formulation is about 0.01% to about 15%. In additional embodiments, the wt % of protein is about 0.1% to about 5%, or about 1% to about 3%.

In certain embodiments, the protein included in the ophthalmic or aqueous formulation is a hydrophobic protein. When referring to a hydrophobic protein, it is understood that the protein may have a “net” hydrophobicity, this is, overall, the protein is more hydrophobic than hydrophilic. Net hydrophobicity is determined using a hydropathic index of amino acids. For example, each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In this example, the more positive values are more hydrophobic. (For example, see Kyte et al., A simple method for displaying the hydropathic character of a protein., J. Mol. Biol. (1982) 157(1):105-32, incorporated herein by reference).

Hydrophobic proteins are those that have a positive total hydropathic index after the following operation: each amino acid in the polypeptide chain is converted to its respective index value and the values are summed to yield a total hydropathic index. The hydrophobic/non-hydrophobic nature of polypeptides and peptides can likewise be determined. It is understood that certain proteins and polypeptides may have regions that are hydrophobic and that these regions interfere with analysis or usefulness of the molecules, for example, MALDI MS. In these cases, the hydropathic index for the region is of interest and is determined. In certain cases, the region will comprise consecutive amino acids and in other cases the region will comprise a hydrophobic surface brought together by higher order folding of the polypeptide chain (such as, tertiary structure).

In some embodiments, the protein is a small fibrous protein (i.e., having little or no tertiary structure) comprising an average molecular weight of about 10 kDa to about 50 kDa, about 10 kDa to about 35 kDa, 15 kDa to about 35 kDa, about 15 kDa to about 30 kDa, about 15 kDa to about 25 kDa, about 16 kDa to about 23 kDa, or about 18 kDa to about 22 kDa. In some embodiments, the protein comprises less than 8% serine amino acid residues. In other embodiments, the protein comprises less than 7.5% serine amino acid residues, less than 7% serine amino acid residues, less than 6.5% serine amino acid residues, or less than 6% serine amino acid residues.

In some embodiments, the protein comprises greater than 46% glycine amino acids, relative to the total amino acid content of the protein. In other embodiments, the protein comprises greater than 46.5% glycine amino acids, greater than 47% glycine amino acids, greater than 47.5% glycine amino acids, or greater than 48% glycine amino acids.

In some embodiments, the protein comprises greater than 30% alanine amino acids, relative to the total amino acid content of the protein. In other embodiments, the protein comprises greater than 30.5% alanine amino acids, greater than 31% alanine amino acids, greater than 31.5% alanine amino acids, greater than 32% alanine amino acids, greater than 32.5% alanine amino acids, greater than 33% alanine amino acids, or greater than 33.5% alanine amino acids.

In certain embodiments, the protein comprises 46.5% to 48% glycine amino acids and 30% to 33.5% alanine amino acids relative to the total amino acid content of the protein.

In other embodiments, the protein comprises greater than 46% glycine amino acids and greater than 30% alanine amino acids relative to the total amino acid content of the protein.

Exemplary proteins for use in a formulation may be characterized based on their features in solution. For example, when an exemplary protein is present, the resultant formulation may have an aqueous viscosity of less than 4 cP as a 10% w/w solution in water. In various embodiments, the formulation has an aqueous viscosity of less than 10 cP as a 15% w/w solution in water or an aqueous viscosity of less than 10 cP as a 24% w/w solution in water.

In preferred embodiments, the protein is a fibroin-derived protein (e.g., SDP-4). In various embodiments, the wt. % of fibroin-derived protein in the formulation is about 0.01% to about 15%. In additional embodiments, the wt % of fibroin-derived protein is about 0.1% to about 5%, or about 1% to about 3%. Thus, in preferred embodiments that include a protein, the disclosure provides a formulation comprising a fibroin-derived protein wherein the primary amino acid sequences of the fibroin-derived protein composition differ from native fibroin by at least 4% with respect to the absolute values of the combined differences in amino acid content of serine, glycine, and alanine; cysteine disulfide bonds between the fibroin heavy and fibroin light protein chains of the fibroin-derived protein are reduced or eliminated; the protein composition has a serine content that is reduced by greater than 25% compared to native fibroin, wherein the serine content is at least about 5%; and the average molecular weight of the fibroin-derived protein composition is less than 35 kDa and greater than 15 kDa; and a buffering agent, polysorbate-80, and one or more osmotic agents; wherein the formulation has a pH of 4.5 to 6.0 and a particulate count of 50/mL or less after a storage period of greater than 12 weeks at 4° C. to 40° C. with respect to particulates having a diameter of 10 micrometers or more.

In various other embodiments, the fibroin-derived protein composition is Silk Derived Protein-4 (SDP-4) having an average molecular weight of about 15 kDa to about 35 kDa, or about 18 kDa to about 22 kDa, and the pH of the formulation is 5.2 to 5.8. In other embodiments, the pH is about 5.0 to about 6.0.

One certain preferred embodiment is an aqueous formulation for use in the treatment of eye related conditions that stabilizing a protein in solution comprising about 0.1 wt. % to about 3 wt. % fibroin-derived protein wherein the primary amino acid sequences of the fibroin-derived protein differs from native fibroin by at least 6% with respect to the absolute values of the combined differences in amino acid content of serine, glycine, and alanine; cysteine disulfide bonds between the fibroin heavy and fibroin light protein chains of the fibroin-derived protein are reduced or eliminated; the fibroin-derived protein comprises greater than 46% glycine amino acids and greater than 30% alanine amino acids; the fibroin-derived protein has a serine content that is reduced by greater than 40% compared to native fibroin protein such that the fibroin-derived protein comprises less than 8% serine amino acids; and the average molecular weight of the fibroin-derived protein is about 15 kDa to about 35 kDa; and polysorbate-80, about 10 millimolar to about 50 millimolar acetate buffer, and an osmotic agent; wherein the formulation has a pH of 5.2 to 5.8, an osmolality of 175 mOsm/kg to 185 mOsm/kg, and a particulate count of 50/mL or less after a storage period of greater than 12 weeks at 4° C. to 40° C. with respect to particulates having a diameter of 10 micrometers or more.

In various embodiments, the acetate buffer comprises about 0.2 wt. % to about 0.3 wt. % sodium acetate and about 0.01 wt. % to about 0.03 wt. % acetic acid. In other embodiments, the osmotic agent comprises about 0.6 wt. % to about 0.9 wt. % dextrose and about 0.6 wt. % to about 0.9 wt. % magnesium chloride. In additional embodiments, the wt. % of polysorbate-80 is about 0.05% to about 0.1%.

Additionally, this disclosure provides a method for treating an ophthalmic disease comprising administering an effective amount of the formulation disclosed herein to a subject having an ophthalmic disease, thereby treating the ophthalmic disease. In some embodiments, the ophthalmic disease is dry eye syndrome.

In view of the foregoing, the disclosure provides for the following embodiments:

1. An ophthalmic formulation comprising one or more buffering agents, a surfactant, and one or more osmotic agents; wherein the formulation has a pH of 4.5 to 6.0 and the formulation is capable of maintaining a protein in solution for a period greater than 4 weeks without gelation, and is capable of maintaining a particulate count of 50/mL or less after a storage period of greater than 12 weeks at 4° C. to 40° C. with respect to particulates having a diameter of 10 micrometers or more. The formulation can include or exclude a protein composition such as SDP-4.

2. The formulation of clause 1 wherein the osmolality of the formulation is about 160 mOsm/kg to about 280 mOsm/kg.

3. The formulation of clause 2 wherein the osmolality of the formulation is about 175 mOsm/kg to about 185 mOsm/kg.

4. The formulation of clause 1 wherein the one or more buffering agents comprise histidine, acetate, glutamate, or a combination thereof.

5. The formulation of clause 4 wherein the formulation has a buffer concentration of about 10 millimolar to about 50 millimolar, or the concentration of each of the one or more osmotic agents in the formulation is about 30 millimolar to about 40 millimolar.

6. The formulation of clause 4 wherein the one or more buffering agents comprise about 0.1 wt. % to about 1.0 wt. % sodium acetate and about 0.01 wt. % to about 0.1 wt. % acetic acid.

7. The formulation of clause 1 wherein the one or more osmotic agents comprise a monosaccharide, an inorganic salt, or a combination thereof.

8. The formulation of clause 7 wherein the one or more osmotic agents comprise mannitol, dextrose, sodium chloride, magnesium chloride, or a combination thereof.

9. The formulation of clause 7 wherein the one or more osmotic agents comprise about 0.10 wt. % to about 2.0 wt. % dextrose and about 0.10 wt. % to about 2.0 wt. % magnesium chloride.

10. The formulation of clause 1 wherein the surfactant is polysorbate 40, polysorbate 60, or polysorbate 80.

11. The formulation of clause 1 wherein the wt. % of the surfactant is about 0.02% to about 1.0%.

12. The formulation of clause 10 wherein the surfactant is polysorbate 80 present at a wt. % of about 0.02% to about 0.5%

13. The formulation of clause 1 wherein the formulation has a pH of 5.2 to 5.8 and an osmolality of 175 mOsm/kg to 185 mOsm/kg.

14. The formulation of any one of clauses 1-13, wherein the formulation further comprises a protein, wherein the wt. % of the protein in the formulation is about 0.01% to about 3%.

15. The formulation of clause 14 wherein the protein is hydrophobic protein.

16. The formulation of clause 15 wherein a primary amino acid sequence of the hydrophobic protein comprises about 33% alanine and about 48% glycine.

17. The formulation of clause 14 wherein the protein is a fibroin-derived protein comprising: a primary amino acid sequences of the fibroin-derived protein composition differ from native fibroin by at least 4% with respect to the absolute values of the combined differences in amino acid content of serine, glycine, and alanine; cysteine disulfide bonds between the fibroin heavy and fibroin light protein chains of the fibroin-derived protein are reduced or eliminated; the fibroin-derived protein has a serine content that is reduced by greater than 25% compared to native fibroin, wherein the serine content is at least about 5%; and the average molecular weight of the fibroin-derived protein in the formulation is less than 35 kDa and greater than 15 kDa.

18. An aqueous formulation comprising about 0.04 wt. % to about 0.1 wt. % polysorbate-80; an acetate buffer comprising about 0.2 wt. % to about 0.3 wt. % sodium acetate and about 0.01 wt. % to about 0.03 wt. % acetic acid; and an osmotic agent comprising about 0.6 wt. % to about 0.9 wt. % dextrose and about 0.6 wt. % to about 0.9 wt. % magnesium chloride; wherein the formulation has a pH of 5.2 to 5.8 and an osmolality of 175 mOsm/kg to 185 mOsm/kg.

19. The aqueous formulation of clause 18 further comprising a protein having a wt. % of about 0.01% to about 3%.

20. The aqueous formulation of clause 19 wherein the protein is a hydrophobic protein having an average molecular weight of less than 35 kDa and greater than 15 kDa.

21. The aqueous formulation of clause 19 or 20 wherein the formulation has a particulate count of 50/mL or less after a storage period of greater than 12 weeks at 4° C. to 40° C. with respect to particulates having a diameter of 10 micrometers or more.

22. An aqueous formulation consisting essentially of: about 0.04 wt. % to about 0.1 wt. % polysorbate-80; an acetate buffer comprising about 0.2 wt. % to about 0.3 wt. % sodium acetate and about 0.01 wt. % to about 0.03 wt. % acetic acid; and an osmotic agent comprising about 0.6 wt. % to about 0.9 wt. % dextrose and about 0.6 wt. % to about 0.9 wt. % magnesium chloride; wherein the formulation has a pH of 5.2 to 5.8 and an osmolality of 175 mOsm/kg to 185 mOsm/kg.

23. An ophthalmic formulation comprising one or more surfactants; one or more osmotic agents; and an acetate buffering system comprising about 0.1 wt. % to about 1.0 wt. % sodium acetate and about 0.01 wt. % to about 0.1 wt. % acetic acid, wherein the buffering system maintains the formulation at a pH of 4.5 to 6.0; and the formulation is capable of maintaining a protein in solution for a period greater than 4 weeks without gelation, and the formulation is capable of maintaining a particulate count of 50/mL or less after a storage period of greater than 12 weeks at 4° C. to 40° C. with respect to particulates having a diameter of 10 micrometers or more, when protein is added to the ophthalmic formulation.

24. The ophthalmic formulation of clause 23 wherein the one or more surfactants is polysorbate 80.

25. The ophthalmic formulation of clause 23 further comprising a protein, wherein the protein is a hydrophobic protein having an average molecular weight of less than 35 kDa and greater than 15 kDa.

26. An ophthalmic formulation consisting essentially of one or more surfactants; one or more osmotic agents; and an acetate buffering system comprising about 0.1 wt. % to about 1.0 wt. % sodium acetate and about 0.01 wt. % to about 0.1 wt. % acetic acid, wherein the buffering system maintains the formulation at a pH of 4.5 to 6.0.

27. An ophthalmic formulation consisting essentially of a silk fibroin-derived protein; one or more surfactants; one or more osmotic agents; and an acetate buffering system comprising about 0.1 wt. % to about 1.0 wt. % sodium acetate and about 0.01 wt. % to about 0.1 wt. % acetic acid, wherein the buffering system maintains the formulation at a pH of 4.5 to 6.0; and the formulation is capable of maintaining a protein in solution for a period greater than 4 weeks without gelation, and the formulation is capable of maintaining a particulate count of 50/mL or less after a storage period of greater than 12 weeks at 4° C. to 40° C. with respect to particulates having a diameter of 10 micrometers or more, when protein is added to the ophthalmic formulation.

28. The ophthalmic formulation of clause 27 wherein the silk-derived protein has an average molecular weight of about 35 kDa to about 15 kDa. 29. The ophthalmic formulation of clause 27 wherein the one or surfactants is polysorbate 80.

30. A method for treating an ophthalmic disease comprising administering an effective amount of the formulation of any one of clauses 1-29 to a subject having an ophthalmic disease, thereby treating the ophthalmic disease. 31. The method of clause 30 wherein the ophthalmic disease is Dry Eye Syndrome.

### Therapeutic Methods

The invention provides for the use of SDP in formulations to reduce inflammation, for example, inflammation on or in the human cornea. Such reduction in inflammation has been demonstrated in both in vitro and in vivo experimental models. Specifically, work was undertaken to show that SDP works to reduce inflammation in human corneal models by inhibiting NF-κB-associated cell signaling pathways, known drivers of inflammation in the body, in which one specific example is dry eye disease. It was found that inhibition of these pathways ultimately led to reduced genetic expression and tissue residence of MMP-9, which is also a known driver of dry eye and ocular inflammation.

The invention thus provides methods for reducing inflammation and for treating wounds, including corneal wounds, comprising the administration of SDP to the site of interest. The methods can include administering a formulation comprising a composition of silk-derived protein (SDP), or molecular fractions thereof, to inflamed tissue, e.g., living animal tissue in a wound. In some embodiments, the subject has an ocular condition that results in inflamed tissue, for example, as in dry eye disease. In some embodiments, the wound is an ocular wound, a surgical wound, an incision, or an abrasion. The ocular wound can be, for example, a corneal wound.

SDP and SDP-4 can thus be used to treat and/or reduce the inflammation caused by conditions such as a wound, infection, or disease. Examples of such conditions include ocular wounds, surgical wounds, incisions, or abrasions. In some cases, the inflammation is caused by an ocular condition, such as, dry eye disease or syndrome, corneal ulcer, corneal erosion, corneal abrasion, corneal degeneration, corneal perforation, corneal scarring, an epithelial defect, keratoconjunctivitis, idiopathic uveitis, corneal transplantation, age-related macular degeneration (AMD, wet or dry), diabetic eye conditions, blepharitis, glaucoma, ocular hypertension, post-operative eye pain and inflammation, posterior segment neovascularization (PSNV), proliferative vitreoretinopathy (PVR), cytomegalovirus retinitis (CMV), endophthalmitis, choroidal neovascular membranes (CNVM), vascular occlusive diseases, allergic eye disease, tumors, retinitis pigmentosa, eye infections, scleritis, ptosis, miosis, eye pain, mydriasis, neuralgia, cicatrizing ocular surface diseases, ocular infections, inflammatory ocular diseases, ocular surface diseases, corneal diseases, retinal diseases, ocular manifestations of systemic diseases, hereditary eye conditions, ocular tumors, increased intraocular pressure, herpetic infections, ptyrigium (scleral tumor), wounds sustained to ocular surface, post-photorefractive keratotomy eye pain and inflammation, thermal or chemical burns to the cornea, scleral wounds, keratoconus and conjunctival wounds. In some embodiments, the inflammation and/or ocular condition is caused by aging, an autoimmune condition, trauma, infection, a degenerative disorder, endothelial dystrophies, and/or surgery. In one specific example, SDP or SDP-4 is used in a formulation to treat dry eye syndrome.

The following Examples are intended to illustrate the above inventions and should not be construed as to narrow its scope. One skilled in the art will readily recognize that the Examples suggest many other ways in which the inventions could be practiced. It should be understood that numerous variations and modifications may be made while remaining within the scope of the inventions.

### EXAMPLES

### Example 1. Preparation of OTC and Anti-Inflammatory Eye Drop Formulations

An eye drop composition can be prepared to take advantage of the therapeutic properties of SDP to treat the ocular system because of disease or injury. SDP molecules can be optionally isolated based on molecular weights or used as a whole composition. A composition of protein molecules of low average molecular weight, such as less than about 35 kDa and greater than about 15 kDa, may be prepared and is referred to as SDP-4. A second composition of protein molecules that includes all molecular weights of the SDP composition or molecules more than about 40 kDa can also be prepared. Each composition can include water, at least one buffer or buffer system (e.g., phosphate buffered saline (PBS), citrate, borate, Tris, 4-(2-hydroxyethyl)-1-piperazineethanesulfonic acid (HEPES)), optionally at least one preservative (e.g., perborate, benzalkonium chloride (BAK)) and optionally at least one additional excipient, surfactants, stabilizers or salt (e.g., sulfanilic acid, trehalose, glycerin, ethylenediaminetetraacetic acid (EDTA), polyethylene glycol (PEG), mannitol, polysorbate, sodium chloride (NaCl), magnesium chloride (MgCl₂), calcium chloride (CaCl₂)), or lithium bromide (LiBr)).

The eye formulation containing the first compositions above can be applied as a therapeutic product to a dry eye disease patient, a wounded patient, or a surgical wound of an otherwise healthy patient (e.g., for post-refractive or cataract surgery). The disease or injury can be monitored over time for inflammation and wound closure rate, and for patient comfort and pain assessment. The second compositions can be used in over-the-counter products, such as an artificial tears eye drop product, as a protein excipient to help with enhancing formulation wetting, spreading, and patient comfort.

An example of an eye drop formulation would contain as low as 0.1% wt./vol. SDP-4 or SDP to as high as 10% wt./vol. SDP-4 or SDP. The SDP-4 or SDP material would be dissolved into purified water, where a buffer system such as citric acid buffer, Tris buffer, PBS buffer, or borate buffer would be created in a 1 mmol to 1,000 mmol concentration. Additional excipient ingredients may be added to the formulation. A surfactant, such as polysorbate, could be added in the range of a 0.01%-0.1% wt./vol. concentration. Stabilizing sugar molecules can be added, such as trehalose, dextrose, or sucrose, at concentrations ranging from 10 mmol-500 mmol. Demulcent molecules can be added as ocular lubricants, such as PEG, carboxy methyl cellulose, hypromellose, hydroxypropyl methylcellulose, or glycerin, at concentrations ranging from 0.1%-2.0% wt./vol. Salts may also be added to reduce molecular interactions and stabilize the formulation, such as NaCl, MgCl₂, CaCl₂), or LiBr, at concentration ranging from 10 mmol-500 mmol. Amino acid molecules can be added as stabilizing agents, such as L-glutamine or L-arginine, at concentrations ranging from 10 mmol-500 mmol. Chelating agents can be added as stabilizing agents, such as EDTA, at concentrations ranging from 0.01%-0.1% wt./vol. Antimicrobial agents can be added to the formulation, such as perborate or BAK, at concentrations of up to 0.015% wt./vol.

In Table 1 below are some example base formulations that have been produced containing the SDP-4 and/or SDP molecules, in which additional additives or excipients can be added to enhance formulation applications described above:

TABLE 1
Examples of Base Formulations

| | Composition | | | | |
|---|---|---|---|---|---|
| Ingredient | 1 | 2 | 3 | 4 | 5 |
| SDP-4 or SDP | 5 or 10 g | 5 or 10 g | 5 or 10 g | 5 or 10 g | 5 or 10 g |
| Phosphate | 10 mmol | — | — | — | — |
| NaCl | 137 mmol | — | — | — | — |
| KCl | 2.7 mmol | — | — | — | — |
| Citric Acid | — | 82 mmol | 8 mmol | — | — |
| Trisodium | — | 18 mmol | 92 mmol | — | — |
| Citrate | | | | | |
| Tris | — | — | — | 7.02 g | 0.76 g |
| Hydrochloric | | | | | |
| Acid | | | | | |
| Tris Base | — | — | — | 0.67 g | 5.47 g |
| Water | 1 L | 1 L | 1 L | 1 L | 1 L |
| pH | 7.4 | 3.0 | 6.2 | 7.2 | 9.0 |

SDP, or an SDP fraction such as SDP-4, can also be added to known eye formulations such as commercial and prescription eye drops and ointments to improve wetting and patient comfort. Examples of ophthalmic solutions that SDP or SDP-4 can be added to include brimonidine tartrate, brimonidine tartrate/timolol maleate, alcaftadine, bimatoprost, cyclosporine, gatifloxacin, ketorolac tromethamine, or lifitegrast ophthalmic solutions. Examples of other formulations that SDP or SDP-4 can be added to are described in U.S. Pat. Nos. 5,468,743; 5,880,283; 6,333,045; 6,562,873; 6,627,210; 6,641,834; 6,673,337; 7,030,149; 7,320,976; 7,323,463; 7,351,404; 7,388,029; 7,642,258; 7,842,714; 7,851,504; 8,008,338; 8,038,988; 8,101,161; 8,133,890; 8,207,215; 8,263,054; 8,278,353; 8,299,118; 8,309,605; 8,338,479; 8,354,409; 8,377,982; 8,512,717; 8,524,777; 8,541,463; 8,541,466; 8,569,367; 8,569,370; 8,569,730; 8,586,630; 8,629,111; 8,632,760; 8,633,162; 8,642,556; 8,648,048; 8,648,107; 8,664,215; 8,685,930; 8,748,425; 8,772,338; 8,858,961; 8,906,962; and 9,248,191, and U.S. Pat. Nos. 7,314,938; 7,745,460; 7,790,743; 7,928,122; 8,084,047; 8,168,655; 8,367,701; 8,592,450; 8,927,574; 9,045,457; 9,085,553; 9,216,174; 9,353,088; and 9,447,077.

### Example 2. Pre-Formulation Studies

Bombyx mori silkworm cocoons were purchased from Shanghai Yu Yuan Company. Raw silk fibers were extracted using a 0.3% wt./wt. Na₂CO₃(J.T. Baker, USP Grade) solution for 75 minutes at 95° C. and then rinsed thoroughly with purified water (SilkTech Biopharmaceuticals) for 20 minutes. The rinse cycle was then repeated an additional three times to ensure that all residual Na₂CO₃ and the extracted glue-like sericin proteins have been washed away. The degummed extracted silk fibers were then pressed to remove excess water and then dried at 70° C. for 16 hours in a convection oven.

The dried extracted silk fibers were then solubilized in 54% wt./wt. lithium bromide (LiBr) solution (FMC Lithium, Inc) at a ratio of 4× LiBr volume per gram of extracted fiber in a process called Reaction. This step was performed at various solubilization times under temperatures of 121° C. and 15 psi, yielding an intermediate solution called SDP/LiBr intermediate. This intermediate solution was then fractionated using a Tangential Flow Filtration (TFF) 30 kDa Sartorius Hydrosart cut off filter and retaining all fractions below 30 kDa, Next, this fraction was filtered using a ITT 10 kDa Sartorius Hydrosart cut off filter and retaining all fractions above 10 kDa. The resulting product is Silk Derived Protein-4 (SDP-4), All Sartorius Hydrosart cut off filters were purchased from Sartorius Stedim.

During the initial formulation developmental phase (Table 2-6), stock solution of various buffers, salts, sugars and surfactants were created. These stock solutions were then added directly into SDP-4 (Reacted for 30 minutes) and diluted with purified water until the desired. concentration of excipient and SDP-4 was reached. This solution was mixed until fully dissolved and then filtered with a 0.2 μm polyethersulfone filter (VWR) and placed in 50 mL polypropylene conicals (VWR). The containers containing the formulation was then placed in a stability chamber under conditions of 40° C. and 75% relative humidity and monitored for stability. Table 2 below shows the chemical and manufacturer of the initial formulation development. Each item in the pre-formulation study (Table 3-6) was evaluated using qualitative analyses. The acceptance criteria for a passing formulation is that it must not gel and be essentially free of visible particulates. The acceptance criteria were not met for each item within the pre-formulation study and therefore failed the pre-formulation study screening process.

TABLE 2
Excipients and their manufacturer

| Excipient | Manufacturer | Excipient | Manufacturer |
|---|---|---|---|
| Magnesium Chloride | VWR | Sodium Citrate | VWR |
| Calcium Chloride | Millipore | Tween 20 | VWR |
| Sodium Chloride | Carolina | Tween 80 | VWR |
| Phosphate Buffered | VWR | TRITON | Millipore |
| Saline | | Trehalose | Swanson |
| Tris HCl | VWR | Glycerin | Spectrum |
| TRIS NaOH | VWR | EDTA | BDH |
| Sodium Borate | VWR | PEG-400 | Sigma |
| Boric Acid | BDH | D-Mannitol | VWR |
| Citric Acid | VWR | L-Glutamine | VWR |
| L-Arginine | VWR | | |
| Bovine Serum | VWR | | |
| Albumin | | | |

TABLE 3
Effect of Salt on SDP-4 (30 Minute Reaction)

| SDP-4 | | Salt | Days | |
|---|---|---|---|---|
| Concentration | Salt | Concentration | until Aggregation/ | |
| (% wt./wt.) | Added | (Molarity) | Gelation | Description |
| 1% | Control | — | 6 | Small Aggregates. |
| | | | | High Count (>6 |
| | | | | particulates per mL) |
| 1% | MgCl2 | 0.05 | 6 | Small Aggregates. |
| | | | | High Count (>6 |
| | | | | particulates per mL) |
| 1% | CaCl2 | 0.05 | 6 | Large Aggregates. |
| | | | | Very high count (>10 |
| | | | | particulates per mL) |
| 1% | NaCl | 0.05 | 6 | Large Aggregates. |
| | | | | Medium Count (>4 |
| | | | | particulates per mL) |
| 5% | Control | — | 35 | Gelation |
| 5% | MgCl2 | 0.05 | 35 | Gelation |
| 5% | CaCl2 | 0.05 | 35 | Gelation |
| 5% | NaCl | 0.05 | 35 | Gelation |

TABLE 4
Effect of Buffers on SDP-4 (30 Minute Reaction)

| SDP-4 | | | Days | |
|---|---|---|---|---|
| Concentration | | Buffer | until Aggregation/ | |
| (% wt./wt.) | Buffers | Concentration | Gelation | Description |
| 5% | PBS | 0.01M | 7 | Gelation |
| | (pH: 7.2) | | | |
| 5% | Tris | 0.1M | 21 | Gelation |
| | (pH: 7.2) | | | |
| 1% | Sodium | 0.05M | 9 | Small aggregates. |
| | Borate | | | High Count (>6 |
| | (pH: 7.2) | | | particulates per |
| | | | | mL) |
| 1% | Tris | 0.05M | 2 | Small aggregates. |
| | (pH: 7.2) | | | Medium Count |
| | | | | (>4 particulates |
| | | | | per mL) |
| 1% | Citric Acid | 0.1M | 7 | Gelation |
| | (pH: 5.9) | | | |

TABLE 5
Effect of Surfactants on SDP-4 (30 Minute Reaction)

| SDP-4 | | | Days until | |
|---|---|---|---|---|
| Concentration | Additives/ | Additive/Buffer | Aggregation/ | Description of |
| (% wt./wt.) | Buffer | Concentration | Gelation | Aggregates |
| 1% | Tween 20 | 1% wt./wt. | 2 | High Count. Shard |
| | | | | like aggregates |
| | | | | (>6 particulates per |
| | | | | mL) |
| 1% | Tris | 0.05M | 2 | Very high count. |
| | Tween 20 | 1% wt./wt. | | Shard like aggregates |
| | | | | (>10 particulates per |
| | | | | mL) |
| 1% | Tris | 0.05M | 5 | Low count. Small |
| | TRITON | 1% wt./wt. | | aggregates |
| | | | | (~2 particulates mL) |
| 1% | Sodium Borate | 0.05M | 2 | Very high count. |
| | Tween 20 | 1% wt./wt. | | Shard like aggregates |
| | | | | (>10 particulates per |
| | | | | mL) |
| 1% | Sodium Borate | 0.05M | 9 | Low count. Small |
| | TRITON | 1% wt./wt. | | aggregates |
| | | | | (~2 particulates mL) |
| 1% | Tween 20 | 0.10% wt./wt. | 14 | Low count. Shard like |
| | | | | aggregates |
| | | | | (~2 particulates mL) |
| 1% | Tween 80 | 0.10% wt./wt. | 14 | Low count. Shard like |
| | | | | aggregates |
| | | | | (~2 particulates mL) |

TABLE 6
Effect of Additives on SDP-4 (30 Minute Reaction)

| SDP-4 | | | Days until | |
|---|---|---|---|---|
| Concentration | | Additive/Buffer | Aggregation/ | Description of |
| (% wt./wt.) | Additives/Buffer | Concentration | Gelation | Aggregates |
| 5% | Trehalose | 0.05M | 48 | Gel |
| 1% | Trehalose | 0.05M | 6 | Large Aggregates. Very |
| | | | | High Count |
| | | | | (>10 particulates per |
| | | | | mL) |
| 5% | Glycerin | 1% wt./wt. | 41 | Gel |
| 1% | EDTA | 0.10% wt./wt. | 5 | Large Aggregates. |
| | | | | Medium Count |
| | | | | (>4 particulates per mL) |
| 1% | PEG-400 | 1% wt./wt. | 5 | Large Aggregates. Low |
| | | | | Count |
| | | | | (~2 particulates mL) |
| 1% | D-Mannitol | 1% | 9 | Small and Large |
| | | | | Aggregates. Very High |
| | | | | Count |
| | | | | (>10 particulates per |
| | | | | mL) |
| 1% | Tris | 0.05M | 5 | Medium Aggregates. |
| | PEG-400 | 1% wt./wt. | | High Count |
| | | | | (>6 particulates per mL) |
| 1% | Tris | 0.05M | 9 | Small Aggregates. Low |
| | PEG-8000 | 1% wt./wt. | | Count |
| | | | | (~2 particulates mL) |
| 1% | Tris | 0.05M | 9 | Medium Aggregates. |
| | Glycerin | 1% wt./wt. | | High Count |
| | | | | (>6 particulates per mL) |
| 1% | Tris | 0.05M | 9 | Large Aggregates. Low |
| | EDTA | 0.10% wt./wt. | | Count |
| | | | | (~2 particulates mL) |
| 1% | Tris | 0.05M | 5 | Medium Aggregates. |
| | D-Mannitol | 1% wt./wt. | | High Count |
| | | | | (>6 particulates per mL) |
| 1% | Sodium Borate | 0.05M | 9 | Medium Aggregates. |
| | PEG-400 | 1% wt./wt. | | High Count |
| | | | | (>6 particulates per mL) |
| 1% | Sodium Borate | 0.05M | 9 | Small aggregates. Low |
| | PEG-8000 | 1% wt./wt. | | Count |
| | | | | (~2 particulates mL) |
| 1% | Sodium Borate | 0.05M | 5 | Small aggregates. High |
| | Glycerin | 1% wt./wt. | | count |
| | | | | (>6 particulates per mL) |
| 1% | Sodium Borate | 0.05M | 5 | Large aggregates. Low |
| | EDTA | 0.01% wt./wt. | | Count |
| | | | | (~2 particulates mL) |
| 1% | Sodium Borate | 0.05M | 9 | Medium Aggregates. |
| | D-Mannitol | 1% wt./wt. | | High Count |
| | | | | (>6 particulates per mL) |

TABLE 7
Effect of Amino Acids and Protein on SDP-4 (30 Minute Reaction)

| SDP-4 | | | Days until | |
|---|---|---|---|---|
| Concentration | Additives | Additive | Aggregation/ | |
| (% wt./wt.) | Added | Concentration | Gelation | Description |
| 1% | L-Arginine | 500 mM | 46 | High Count |
| | | | | Aggregation |
| | | | | (>6 particulates per |
| | | | | mL) |
| 1% | L-Arginine | 50 mM | 46 | High Count |
| | | | | Aggregation |
| | | | | (>6 particulates per |
| | | | | mL) |
| 1% | L-Glutamine | 50 mM | 46 | Gelation |
| 1% | L- | 50 mM/ | 46 | High Count |
| | Glutamine/L- | 50 mM | | Aggregation |
| | Arginine | | | (>6 particulates per |
| | | | | mL) |
| 1% | Bovine Serum | 0.1% wt./wt. | 46 | Low Count |
| | Albumin | | | Aggregation |
| | | | | (~2 particulates per |
| | | | | mL) |

### Example 3. Effect of pH and Temperature on SDP-4

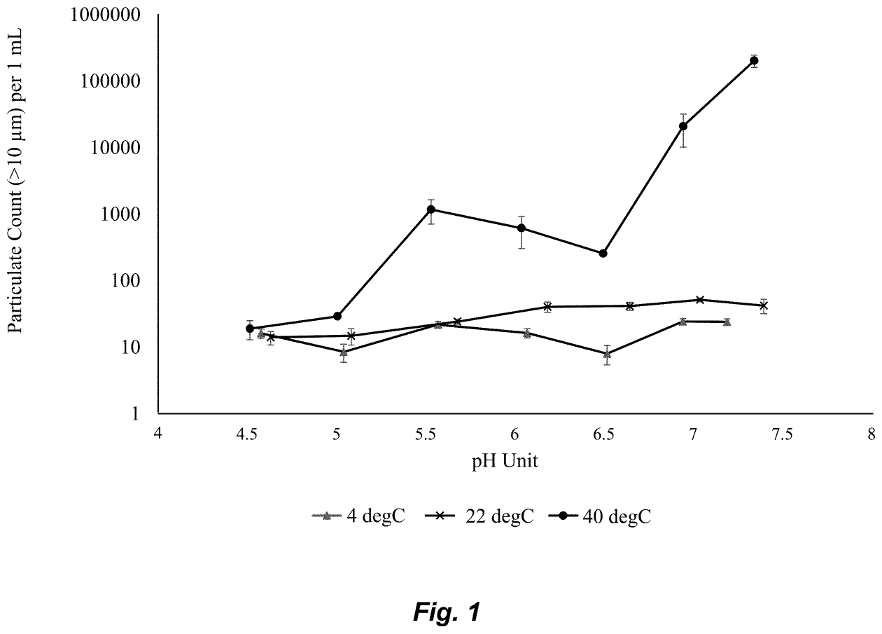

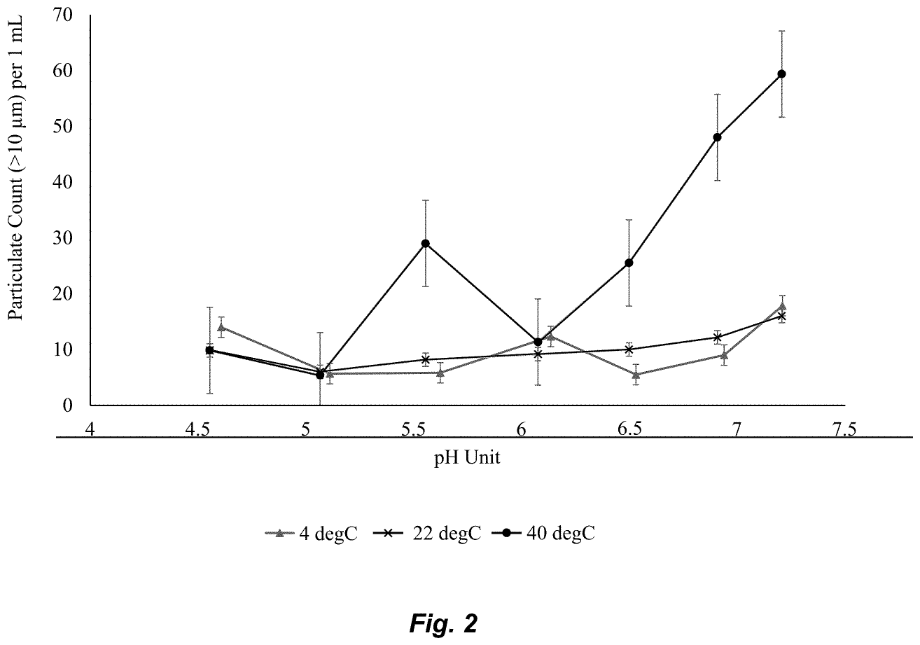

Dried Extracted Fiber was reacted on the benchtop reactor for 30 or 200 minutes. The intermediate was further processed using TFF with 30 kDa and 10 kDa Sartorius Hydrosart filters resulting in SDP-4 (30-minute reaction) and SDP-4 (200-minute reaction). These two test articles were then titrated to the desired pH using 1144 hydrochloric acid (Lab Chem). The samples were then diluted to a concentration of 1% wt./wt. and then filtered using a polyethersulfone filter (VWR) and then aliquoted into 50 mL polypropylene conicals. These conicals containing the SDP-4 with various reaction time and pH were placed in stability chambers under conditions of 4° C., 25° C., and 40° C. At the specified time points, samples were taken out of the stability chambers and analyzed for pH and particulate matter using an Orion Versa Star pH meter and Coulter Particulate Counter, respectively. FIG. 1 shows the summary graph of 30-minute reacted SDP-4 under various pH and temperature conditions. FIG. 2 shows the summary graph of 200-minute reacted SDP-4 under these same conditions. A comparison between 30 minute and 200-minute reaction shows that formation of aggregates is substantially reduced with increased SDP reaction time.

Flocculation of SDP-4 protein occurs below a pH of 5.0. For this study, the flocculants were removed using a combination of centrifugation and filtration. The retained SDP-4 supernatant and filtrate was then used in the study. As seen in FIG. 2, within a pH range of 4.5-6.0, the fewest number of particulates were formed. Above a pH of 5.0 and at 40° C. conditions, subvisible and visible particulates increased. Collectively, this study demonstrates that physical stability of SDP-4 decreases with increasing pH, increasing storage temperature, and shorter reaction time.

### Example 4. Effect of pH and Temperature in Citric-Acid Buffer Formulation

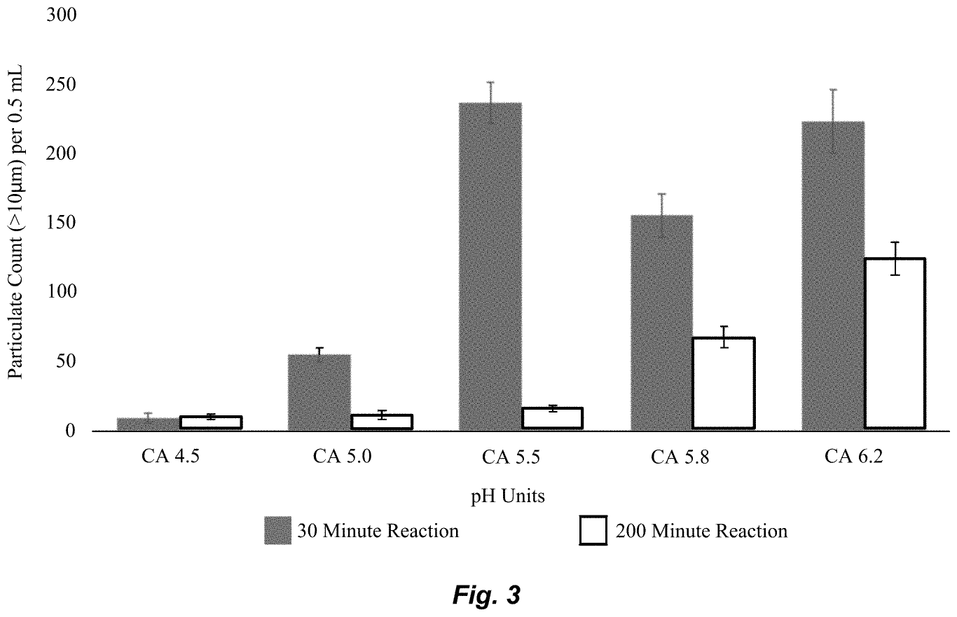

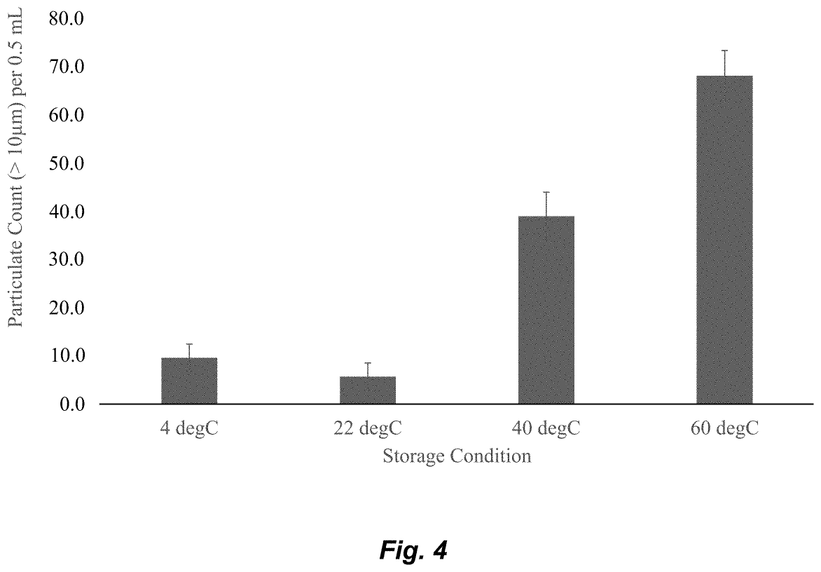

Silk Derived Protein-4 (30 or 200-minute benchtop reaction) was added to citric acid buffer. The citric acid buffer consists of citric acid (VWR) and sodium citrate (VWR). By adding different ratios of citric acid and sodium citrate, the desired pH was obtained. SDP-4 was added to the citric acid buffer and then diluted with purified water to reach a final concentration of 50 nM citric acid buffer and 1% wt./wt. SDP-4 concentration. The formulations were then filtered using a polyethersulfone filter (VWR) and then aliquoted into 50 mL polypropylene conicals (VWR). These conicals containing SDP-4 with various reaction time and citric acid formulation pH were placed in stability chamber under conditions of 40° C. After two weeks, samples were measured for particulates using a Coulter particulate counter. FIG. 3 shows the impact of reaction times and pH on the formation of particulates, and FIG. 4 demonstrates the impact of storage temperature on the particulate formation. These studies demonstrate that SDP-4 physical stability decreases with increasing pH, increasing storage temperature, and shorter reaction time in a citrate-buffered formulation.

### Example 5. Compatibility of SDP-4 and Container Closure Systems

Developmental batches of SDP-4 were manufactured at SilkTech Biopharmaceuticals and stored in various container closure to study the effects of compatibility of SDP-4 and the container closure.

Three types of container closures were chosen for this study: low density polyethylene (LDPE), glass, and polypropylene (PP). Container closures were washed with purified water to remove particulates from the manufacturing process. A developmental batch of SDP-4 with a concentration of 5.79% wt./wt. was diluted with purified water by adding 2954.6 g of Purified water to 617.0 g of SDP-4. Containers were filled with serological pipettes to 50% and 100% of the volume to analyze headspace. After filling, samples were stored under accelerated conditions of 40° C. and 75% relative humidity (RH). Samples were measured for appearance and particulate matter (using Coulter method) after two weeks. Table 8 shows the details of the materials and equipment used.

TABLE 8
Summary of Materials and Equipment

| | | Product | |
|---|---|---|---|
| Material/Equipment | Manufacturer | Code | Lot Number |
| SDP-4 | Silk | N/A | 1300004-3 |
| | Technologies, | | |
| | Ltd. | | |
| Glass Container Closure | VWR | 66012-044 | 083A02 |
| (22 mL Borosilicate Glass | | | |
| with Phenolic Screw Cap) | | | |
| LDPE Container Closure | Thermo | 2103-0001 | 1196397 |
| (20 mL Nalgene Laboratory | Fischer | | |
| Bottle, Wide Mouth) | | | |
| PP Container Closure | VWR | 89039-662 | 10742-734CC |
| (50 mL High-Performance | | | |
| Centrifuge | | | |
| Tube with Plug Cap) | | | |
| Particle Counter | Beckman | Z2 | EQPT-00172 |
| | Coulter | | |

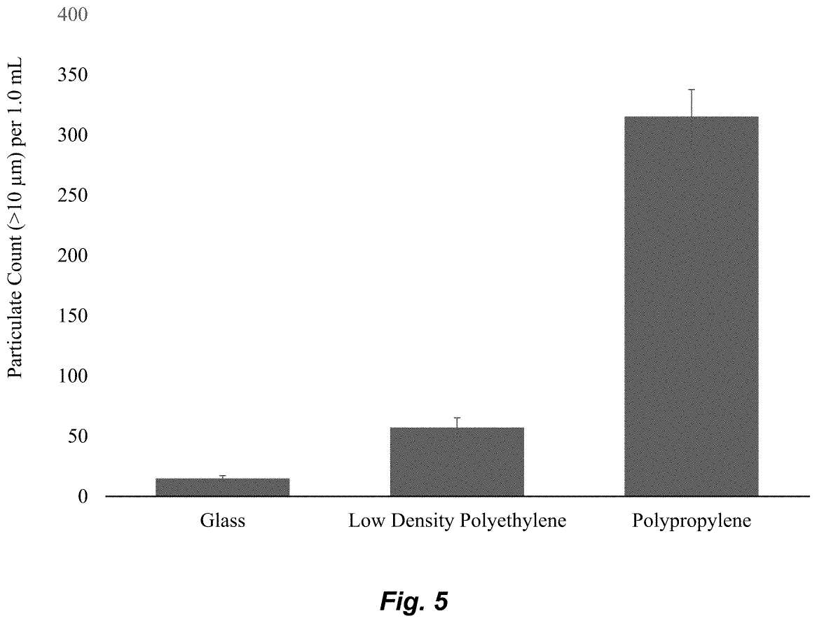

Table 9 shows the results after 2 weeks under conditions of 40° C. and 75% RH. Table 10 shows the summary of results. FIG. 5 shows the particulate count result of average subvisible particulate Count (>10 μm) per mL at 50% of the container volume capacity.

TABLE 9
Results of Tested Closure Systems

| | Appearance | | | | | | |
|---|---|---|---|---|---|---|---|
| | Visible | Particulate Count | | | Standard Deviation | | |
| Packaging | Particles | >10 um | 10 to 25 um | >25 um | >10 um | 10 to 25 um | >25 um |
| Glass 50% | No | 19.3 | 19.0 | 0.3 | 3.3 | 2.8 | 0.5 |
| Glass 50% | No | 10.7 | 10.0 | 0.7 | 1.2 | 1.4 | 0.5 |
| Glass 100% | No | 19.3 | 18.7 | 0.7 | 1.7 | 1.9 | 0.5 |
| Glass 100% | No | 33.3 | 32.3 | 1.0 | 4.5 | 4.6 | 0.8 |
| LDPE 50% | N/A | 59.7 | 58.3 | 1.3 | 7.7 | 7.0 | 1.2 |
| LDPE 50% | N/A | 54.7 | 52.0 | 2.7 | 8.4 | 6.7 | 1.7 |
| LDPE 100% | N/A | 55.3 | 54.7 | 0.7 | 22.8 | 21.9 | 0.9 |
| LDPE 100% | N/A | 36.7 | 35.7 | 1.0 | 4.9 | 4.9 | 0.0 |
| PP 50% | Yes | 210.7 | 207.7 | 2.7 | 28.8 | 28.1 | 0.9 |
| PP 50% | Yes | 420.0 | 414.3 | 5.7 | 16.1 | 16.4 | 1.9 |
| PP 100% | No | 248.0 | 245.0 | 3.0 | 7.1 | 5.7 | 1.4 |
| PP 100% | No | 276.0 | 271.7 | 4.3 | 11.9 | 9.0 | 2.9 |

TABLE 10
Summary of Results of Tested Closure System

| | Appearance/ | Average Subvisible |
|---|---|---|
| | Visible | Particulate |
| Packaging Type | Particulates | Count (10 to 25 μm) |
| Glass 50% Full | No | 15 |
| Glass 100% Full | No | 26 |
| LDPE 50% Full | N/A | 55 |
| LDPE 100% Full | N/A | 46 |
| Polypropylene 50% Full | Yes | 311 |
| Polypropylene 100% Full | No | 262 |

Glass showed lowest number of subvisible particulates and no visible particulates or aggregates were observed. Low density polyethylene storage had higher subvisible particulate counts relative to solutions stored in glass. Visible particulates in LDPE were not visually observed due to the opacity of the container closure. Polypropylene storage showed the highest number of subvisible visible particulates of any container closure. Headspace does not seem to be a factor in formation of particulates in glass and LDPE.

Based on the data and observations for developmental SDP-4 at 1% wt./wt., glass formed less particulate than the LDPE and polypropylene container closure. Glass was chosen as the primary container for SDP-4 with the understanding that phase appropriate stability study on the SDP-4 will be performed.

### Example 6. Pre-Formulations Assessment and Stability Screen

Pre-formulations of SDP-4 were assessed for stability. The osmolality of solutions was adjusted to 290 mOsm/kg (+10 mOsm/kg) with either sodium chloride or mannitol. The descriptions of the 10× diluent and active formulations (where SDP-4 is labeled active pharmaceutical ingredient, API-1) are shown in Table 11.

The diluents were diluted 1:10 with milli-Q water, filtered through a 0.2 μm, 25 mm Acrodisc (Pall p/n 4907) and 20 mL aliquoted into 20 cc clear glass serum vials to be used as controls. 20×1 mL of each of the active formulations was filtered through a 0.2 μm, 25 mm Acrodisc (Pall p/n 4907), aliquoted into 20 cc clear glass serum vials and labeled. The 1× diluent and active formulations were placed at 32.5° C. to 40° C.

All samples were checked at the 1, 2- and 4-week time points for appearance. Samples showing “Opalescence” or “Turbidity” had additional “% Transmittance” performed at 500 nm. All non-gelled samples had % T 500 nm performed at weeks 2 and 4.

The acceptance criteria for a passing formulation is that it must not gel, clear and be essentially free of visible particulates.

TABLE 11
Appearance and % T 500 nm after 4 week under 40° C. conditions

| | Time | | % Transmittance |
|---|---|---|---|
| Formulation | point | Appearance | 500 nm |
| (1x) 0.05% Citrate Buffer, pH | 1 week | Clear Colorless Solution. No | N/A |
| 5.5 Diluent | | Opalescence. | |
| BCL633-001-01, Control | | No Visible Particulates. | |
| | 2 weeks | Clear Colorless Solution. No | 101.9% |
| | | Opalescence. | |
| | | No Visible Particulates. | |
| | 4 weeks | Clear, Colorless solution. Some | 99.6% |
| | | small particles | |
| (1x) 0.1% Histidine Buffer, pH | 1 week | Clear Colorless Solution. No | N/A |
| 6.1 Diluent | | Opalescence. | |
| BCL633-001-02, Control | | No Visible Particulates. | |
| | 2 weeks | Clear Colorless Solution. No | 101.6% |
| | | Opalescence. | |
| | | No Visible Particulates. | |
| | 4 weeks | Clear, Colorless solution. Some | 100.0% |
| | | small particles | |
| (1x) 0.2% Sodium Phosphate | 1 week | Clear Colorless Solution. No | N/A |
| Buffer, pH 7.2 Diluent, | | Opalescence. | |
| BCL633-001-03, Control | | No Visible Particulates. | |
| | 2 weeks | Clear Colorless Solution. No | 101.9% |
| | | Opalescence. | |
| | | No Visible Particulates. | |
| | 4 weeks | Clear, Colorless solution. Some | 100.0% |
| | | small particles | |
| (1x) 0.75% Tromethamine | 1 week | Clear Colorless Solution. No | N/A |
| Buffer, pH 8.1 Diluent | | Opalescence. | |
| BCL633-001-04, Control | | No Visible Particulates. | |
| | 2 weeks | Clear Colorless Solution. No | 100.0% |
| | | Opalescence. | |
| | | No Visible Particulates. | |
| | 4 weeks | Clear, Colorless solution. Some | 100.3% |
| | | small particles | |
| | Time | | % Trans. |
| Formulation | point | Appearance | 500 nm |

API and Buffer

| | | | |
|---|---|---|---|
| 1.0% API-1, 0.05% Citrate | 1 week | Clear Colorless Solution. No | N/A |
| Buffer, pH 5.5 | | Opalescence. | |
| BCL633-001-05 | | No Visible Particulates. | |
| | 2 weeks | Slightly Yellow Solution, Some | 97.6% |
| | | Particulates, No Opalescence | |
| | 4 weeks | Clear, Colorless solution. Some small | 96.3% |
| | | particles | |
| 1.0% API-1, 0.1% Histidine | 1 week | Clear Colorless Solution. No | N/A |
| Buffer, | | Opalescence. | |
| pH 6.1 | | No Visible Particulates. | |
| BCL633-001-06 | 2 weeks | Slightly Yellow Solution, Some | 98.9% |
| | | Particulates, No Opalescence | |
| | 4 weeks | Clear, Colorless solution. Small | 93.4% |
| | | particles/globules | |
| 1.0% API-1, 0.2% Sodium | 1 week | Clear Colorless Solution. No | 96.6% |
| Phosphate Buffer, pH 7.2, | | Opalescence. | |
| BCL633-001-07 | | Some Small Particulates. | |
| | 2 weeks | Slightly Yellow Solution. No | 94.6% |
| | | Opalescence, | |
| | | Some Particulates, Viscous solution. | |
| | 4 weeks | Slightly Yellow, Gelled material. | N/A |
| | | Slightly Opalescent. | |
| | | (Sample re-liquefied upon shaking; | |
| | | Slightly Yellow Thick solution.) | |
| 1.0% API-1, 0.75% | 1 week | Clear Colorless Solution. No | 85.9% |
| Tromethamine Buffer, pH 8.1 | | Opalescence. | |
| BCL633-001-08 | | Some Small Particulates. | |
| | 2 weeks | Slightly Yellow Solution, No | 95.6% |
| | | Opalescence, | |
| | | Some Particulates, Viscous solution. | |
| | 4 weeks | Slightly Yellow, Gelled material. | N/A |
| | | Slightly Opalescent. Some small | |
| | | particles. | |
| | | (Sample re-liquefied upon shaking; | |
| | | Slightly Yellow Thick solution.) | |

API, Buffer and Surfactant

| | | | |
|---|---|---|---|
| 1.0% API-1, 0.1% PS-80, | 1 week | Clear Colorless Solution. No | N/A |
| 0.05% Citrate Buffer, pH 5.5 | | Opalescence. | |
| BCL633-001-11 | | No Visible Particulates. | |
| | 2 weeks | Slightly Yellow Solution, No | 100.7% |
| | | Opalescence, | |
| | | No Visible Particulates | |
| | 4 weeks | Slightly Yellow Solution, No | 96.5% |
| | | Opalescence, | |
| | | No Visible Particulates | |
| 1.0% API-1, 0.1% PS-80, 0.1% | 1 week | Clear Colorless Solution. No | N/A |
| Histidine Buffer, pH 6.1, | | Opalescence. | |
| BCL633-001-12 | | No Visible Particulates. | |
| | 2 weeks | Slightly Yellow Solution, No | 92.0% |
| | | Opalescence, | |
| | | No Visible Particulates | |
| | 4 weeks | Slightly Yellow moderately Viscous | 41.9% |
| | | solution. Slightly Opalescent, No | |
| | | particulates. | |
| 1.0% API-1, 0.1% PS-80, 0.2% | 1 week | Clear Colorless Solution. Slightly | 94.7% |
| Sodium Phosphate Buffer, pH | | Opalescent. | |
| 7.2, | | No Visible Particulates. | |
| BCL633-001-13 | 2 weeks | Opalescent, Viscous solution. | 35.0% |
| | | No Visible Particulates. | |
| | 4 weeks | Slightly Yellow moderately Viscous | 10.1% |
| | | solution. Slightly Opalescent, No | |
| | | particulates. | |
| 1.0% API-1, 0.1% PS-80, | 1 week | Clear Colorless Solution. Slight | 81.7% |
| 0.75% Tromethamine Buffer, | | Opalescence. | |
| pH 8.1, | | No Visible Particulates. | |
| BCL633-001-14 | 2 weeks | Opalescent, Viscous solution. | 38.3% |
| | | No Visible Particulates. | |
| | 4 weeks | Slightly Yellow moderately Viscous | 15.2% |
| | | solution. Slightly Opalescent, No | |
| | | particulates. | |
| 1.0% API-1, 1.0% Povidone, | 1 week | Clear Colorless Solution. No | N/A |
| 0.05% Citrate Buffer, pH 5.5 | | Opalescence. | |
| BCL633-001-15 | | No Visible Particulates. | |
| | 2 weeks | Slightly Yellow Solution, No | 97.9% |
| | | Opalescence, | |
| | | Large and Medium | |
| | | Particulates/globules | |
| | 4 weeks | Slightly Yellow Solution. Some | 95.0% |
| | | small/medium sized particles. No | |
| | | Opalescence. | |
| 1.0% API-1, 1.0% Povidone, | 1 week | Clear Colorless Solution. No | N/A |
| 0.1% Histidine Buffer, pH 6.1 | | Opalescence. | |
| BCL633-001-16 | | Large, Globular Particulates. | |
| | 2 weeks | Slightly Yellow Solution, No | 98.0% |
| | | Opalescence, | |
| | | Large and Medium | |
| | | Particulates/globules | |
| | 4 weeks | Slightly Yellow, Gelled material. Some | N/A |
| | | suspended particulates. Slightly | |
| | | Opalescent.(Sample re-liquefied upon | |
| | | shaking; | |
| | | Slightly Yellow, Viscous solution.) | |
| 1.0% API-1, 1.0% Povidone, | 1 week | Clear Solution. Gelled. Slightly | 102.9% |
| 0.2% Sodium Phosphate | | Opalescent. | |
| Buffer, pH 7.2 | | No Visible Particulates. | |
| BCL633-001-17 | | (Sample re-liquefied upon shaking; | |
| | | Clear, Thick solution. Some | |
| | | Opalescence) | |
| | 2 weeks | Slightly Yellow, Gelled material. | N/A |
| | | Opalescent | |
| | | (Sample re-liquefied upon shaking; | |
| | | Thick solution. Appearance cannot be | |
| | | determined. Too many suspended | |
| | | bubbles) | |
| | 4 weeks | Slightly Yellow Gelled material. | N/A |
| | | (Sample re-liquefied upon shaking; | |
| | | Thick solution. Too many bubbles) | |
| 1.0% API-1, 1.0% Povidone, | 1 week | Clear Solution. Gelled. Slightly | 89.4% |
| 0.75% Tromethamine Buffer, | | Opalescent. | |
| pH 8.1 | | No Visible Particulates. | |
| BCL633-001-18 | | (Sample re-liquefied upon shaking; | |
| | | Clear, Thick solution. Some | |
| | | Opalescence) | |
| | 2 weeks | Slightly Yellow, Gelled material. | N/A |
| | | Opalescent | |
| | | (Sample re-liquefied upon shaking; | |
| | | Thick solution. Appearance cannot be | |
| | | determined. Too many suspended | |
| | | bubbles) | |
| | 4 weeks | Slightly Yellow, Gelled material. Some | N/A |
| | | Opalescence. | |
| | | (Sample re-liquefied upon shaking; | |
| | | Slightly Yellow Thick solution. Too | |
| | | many suspended bubbles) | |

API, Buffer, Surfactant and Osmotic Agent

| | | | |
|---|---|---|---|
| 1.0% API-1, 0.86% Sodium | 1 week | Clear Colorless Solution. No | N/A |
| Chloride, 0.1% PS-80, 0.05% | | Opalescence. | |
| Citrate Buffer, | | No Visible Particulates. | |
| pH 5.5 | 2 weeks | Slightly Yellow Solution, No | 94.1% |
| BCL633-001-19 | | Opalescence, | |
| | | No Visible Particulates | |
| | 4 weeks | Slightly Yellow Solution, Opalescent, | 76.0% |
| | | No Visible Particulates | |
| 1.0% API-1, 0.85% Sodium | 1 week | Clear Colorless Solution. No | N/A |
| Chloride, 0.1% PS-80, 0.1% | | Opalescence. | |
| Histidine Buffer, | | No Visible Particulates. | |
| pH 6.1 | 2 weeks | Slightly Yellow Solution, Slightly | 68.3% |
| BCL633-001-20 | | Opalescent, | |
| | | No Visible Particulates | |
| | 4 weeks | Slightly Yellow Solution, Opalescent, | 40.6% |
| | | Some Particulates/globules | |
| 1.0% API-1, 0.76% Sodium | 1 week | Clear Colorless Solution. Slightly | 94.8% |
| Chloride, 0.1% PS-80, 0.2% | | Opalescent. | |
| Sodium Phosphate Buffer, pH | | No Visible Particulates. | |
| 7.2 | 2 weeks | Slightly Yellow Solution, Slightly | 52.3% |
| BCL633-001-21 | | Opalescent, | |
| | | No Visible Particulates | |
| | 4 weeks | Slightly Yellow Solution, Opalescent, | 22.6% |
| | | No Visible Particulates | |
| 1.0% API-1, 0.57% Sodium | 1 week | Clear Colorless Solution. Slightly | 90.7% |
| Chloride, 0.1% PS-80, 0.75% | | Opalescent. | |
| Tromethamine Buffer, pH 8.1 | | No Visible Particulates. | |
| BCL633-001-22 | 2 weeks | Slightly Yellow Solution, Slightly | 52.6% |
| | | Opalescent, | |
| | | No Visible Particulates | |
| | 4 weeks | Slightly Yellow Solution, Opalescent, | 26.8% |
| | | No Visible Particulates | |
| 1.0% API-1, 0.86% Sodium | 1 week | Clear Colorless Solution. No | N/A |
| Chloride, 1.0% Povidone, | | Opalescence. | |
| 0.05% Citrate Buffer, pH 5.5 | | No Visible Particulates. | |
| BCL633-001-23 | 2 weeks | Slightly Yellow Solution, No | 101.7% |
| | | Opalescent, | |
| | | Some Particulates/globules | |
| | 4 weeks | Slightly Yellow Solution, No | 96.2% |
| | | Opalescent, | |
| | | Some Particulates/globules | |
| 1.0% API-1, 0.84% Sodium | 1 week | Clear Colorless Solution. No | N/A |
| Chloride, 1.0% Povidone, 0.1% | | Opalescence. | |
| Histidine Buffer, pH 6.1 | | No Visible Particulates. | |
| BCL633-001-24 | 2 weeks | Slightly Yellow, Gelled material. | N/A |
| | | Slightly Opalescent. | |
| | | Some Particulates. | |
| | | (Sample re-liquefied upon shaking; | |
| | | Slightly Yellow, Thick solution. Slightly | |
| | | Opalescent) | |
| | 4 weeks | Slightly Yellow, Gelled material. | N/A |
| | | Opalescent. Too many bubbles to | |
| | | determine particulates. | |
| | | (Sample re-liquefied upon shaking; | |
| | | Slightly Yellow, Thick solution.) | |
| 1.0% API-1, 0.76% Sodium | 1 week | Clear Solution. Gelled. Slightly | 103.4% |
| Chloride, 1.0% Povidone, 0.2% | | Opalescent. | |
| Sodium Phosphate Buffer, pH | | No Visible Particulates. | |
| 7.2 | | (Sample re-liquefied upon shaking; | |
| BCL633-001-25 | | Clear, Thick solution. Some | |
| | | Opalescence) | |
| | 2 weeks | Slightly Yellow, Gelled material. | N/A |
| | | Slightly Opalescent. | |
| | | No Particulates. | |
| | | (Sample re-liquefied upon shaking; | |
| | | Slightly Yellow, Thick solution, Slightly | |
| | | Opalescent. Too many bubbles to read | |
| | | accurately) | |
| | 4 weeks | Slightly Yellow, Gelled material. | N/A |
| | | Opalescent. Too many bubbles to | |
| | | determine particulates | |
| | | (Sample re-liquefied upon shaking; | |
| | | Slightly Yellow, Thick solution. Too | |
| | | many bubbles to read accurately) | |
| 1.0% API-1, 0.56% Sodium | 1 week | Clear Solution. Gelled. Slightly | 99.7% |
| Chloride, 1.0% Povidone, | | Opalescent. | |
| 0.75% Tromethamine Buffer, | | No Visible Particulates. | |
| pH 8.1 | | (Sample re-liquefied upon shaking; | |
| BCL633-001-26 | | Clear, Thick solution. Some | |
| | | Opalescence) | |
| | 2 weeks | Slightly Yellow, Gelled material. | N/A |
| | | Slightly Opalescent. | |
| | | No Particulates. | |
| | | (Sample re-liquefied upon shaking; | |
| | | Slightly Yellow, Thick solution. Too | |
| | | many bubbles to read accurately) | |
| | 4 weeks | Slightly Yellow, Gelled material. | N/A |
| | | Slightly Opalescent. | |
| | | Too many bubbles to determine | |
| | | particulates. | |
| | | (Sample re-liquefied upon shaking; | |
| | | Slightly Yellow, Thick solution. Too | |
| | | many bubbles to read accurately) | |
| 1.0% API-1, 4.6% Mannitol, | 1 week | Clear Colorless Solution. No | N/A |
| 0.1% PS-80, 0.05% Citrate | | Opalescence. | |
| Buffer, pH 5.5 | | No Visible Particulates. | |
| BCL633-001-27 | 2 weeks | Slightly Yellow Solution, No | 101.6% |
| | | Opalescence, | |
| | | No Visible Particulates | |
| | 4 weeks | Slightly Yellow Solution, No | 95.7% |
| | | Opalescence, | |
| | | Some small Particles. | |
| 1.0% API-1, 4.5% Mannitol, | 1 week | Clear Colorless Solution. No | N/A |
| 0.1% PS-80, 0.1% Histidine | | Opalescence. | |
| Buffer, pH 6.1 | | No Visible Particulates. | |
| BCL633-001-28 | 2 weeks | Slightly Yellow Solution, No | 91.5% |
| | | Opalescence, | |
| | | No Visible Particulates | |
| | 4 weeks | Slightly Yellow Solution, Slightly | 50.5% |
| | | Opalescent, | |
| | | Some small Particles. | |
| 1.0% API-1, 4.1% Mannitol, | 1 week | Clear Colorless Solution. Slightly | 78.9% |
| 0.1% PS-80, 0.2% Sodium | | Opalescent. | |
| Phosphate Buffer, pH 7.2 | | No Visible Particulates. | |
| BCL633-001-29 | 2 weeks | Slightly Yellow Solution, Opalescent, | 43.0% |
| | | No Visible Particulates, Viscous | |
| | 4 weeks | Slightly Yellow Solution, Opalescent, | 15.7% |
| | | No Visible Particulates, Viscous | |
| 1.0% API-1, 3.1% Mannitol, | 1 week | Clear Colorless Solution. Slightly | 92.1% |
| 0.1% PS-80, 0.75% | | Opalescent. | |
| Tromethamine Buffer, pH 8.1 | | No Visible Particulates. | |
| BCL633-001-30 | 2 weeks | Slightly Yellow Solution, Opalescent, | 47.0% |
| | | No Visible Particulates, Viscous | |
| | 4 weeks | Slightly Yellow Solution, Opalescent, | 18.6% |
| | | No Visible Particulates, Viscous | |
| 1.0% API-1, 4.6% Mannitol, | 1 week | Clear Colorless Solution. No | N/A |
| 1.0% Povidone, 0.05% Citrate | | Opalescence. | |
| Buffer, pH 5.5 | | No Visible Particulates. | |
| BCL633-001-31 | 2 weeks | Slightly Yellow Solution, No | 101.0% |
| | | Opalescence, | |
| | | Some Particulates | |
| | 4 weeks | Slightly Yellow Solution, No | 97.3% |
| | | Opalescence, | |
| | | Some Particulates | |
| 1.0% API-1, 4.5% Mannitol, | 1 week | Clear Colorless Solution. No | N/A |
| 1.0% Povidone, 0.1% Histidine | | Opalescence. | |
| Buffer, pH 6.1 | | No Visible Particulates. | |
| BCL633-001-32 | 2 weeks | Slightly Yellow Solution, No | 99.1% |
| | | Opalescence, | |
| | | Some Particulates | |
| | 4 weeks | Slightly Yellow, Gelled material. | N/A |
| | | Slightly Opalescent. | |
| | | No Visible Particulates. | |
| | | (Sample re-liquefied upon shaking; | |
| | | Slightly Yellow, Thick solution.) | |
| 1.0% API-1 4.1% Mannitol, | 1 week | Clear Solution. Gelled. Slightly | 79.0% |
| 1.0% Povidone, 0.2% Sodium | | Opalescent. | |
| Phosphate Buffer, pH 7.2 | | No Visible Particulates. | |
| BCL633-001-33 | | (Sample re-liquefied upon shaking; | |
| | | Clear, Thick solution. Some | |
| | | Opalescence) | |
| | 2 weeks | Slightly Yellow, Gelled material. | N/A |
| | | Slightly Opalescent. | |
| | | No Particulates. | |
| | | (Sample re-liquefied upon shaking; | |
| | | Slightly Yellow, Thick solution. Too | |
| | | many bubbles to read accurately) | |
| | 4 weeks | Slightly Yellow, Gelled material. | N/A |
| | | Slightly Opalescent. | |
| | | Too many bubbles to determine | |
| | | particulates. | |
| | | (Sample re-liquefied upon shaking; | |
| | | Slightly Yellow, Thick solution. Too | |
| | | many bubbles to read accurately) | |
| 1.0% API-1, 3.0% Mannitol, | 1 week | Clear Solution. Gelled. Slightly | 139.5% |
| 1.0% Povidone, 0.75% | | Opalescent. | |
| Tromethamine Buffer, pH 8.1 | | No Visible Particulates. | |
| BCL633-001-34 | | (Sample re-liquefied upon shaking; | |
| | | Clear, Thick solution. Some | |
| | | Opalescence) | |
| | 2 weeks | Slightly Yellow, Gelled material. | N/A |
| | | Slightly Opalescent. | |
| | | No Particulates. | |
| | | (Sample re-liquefied upon shaking; | |
| | | Slightly Yellow, Thick solution. Too | |
| | | many bubbles to read accurately) | |
| | 4 weeks | Slightly Yellow, Gelled material. | N/A |
| | | Slightly Opalescent. | |
| | | Too many bubbles to determine | |
| | | particulates. | |
| | | (Sample re-liquefied upon shaking; | |
| | | Slightly Yellow, Thick solution. Too | |
| | | many bubbles to read accurately) | |

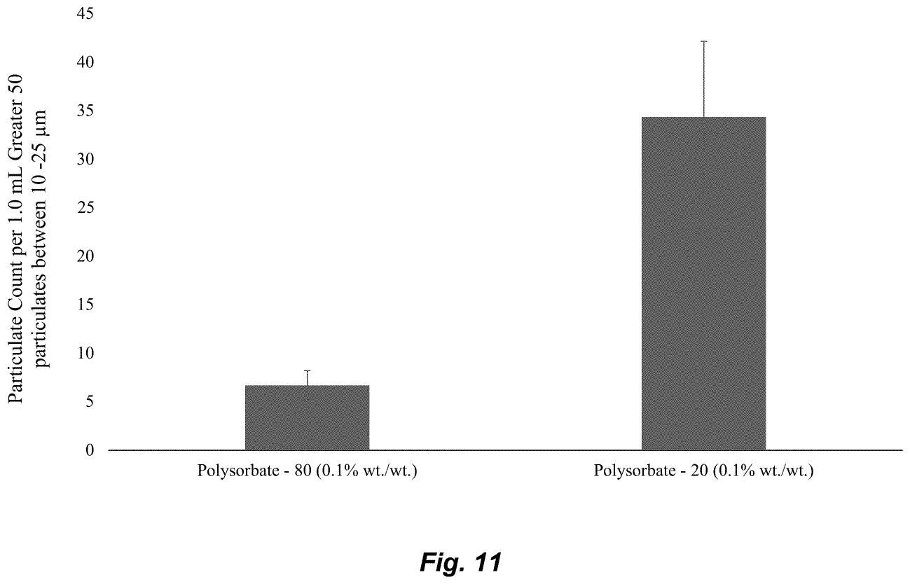

1-Week Observations: The solutions containing povidone and sodium phosphate or tromethamine gelled. Silk Derived Protein-4 (API-1, FIG. 11) prepared with the sodium phosphate, pH 7.2 buffer and the tromethamine, pH 8.1 buffer were the only sample to show opalescence.

2-Week Observations: The solutions containing povidone and sodium phosphate, pH 7.2 or tromethamine pH 8.1 was a firmer gel than at 1 week. The histidine, pH 6.1 buffer with povidone gelled. Overall, most of the solutions started to yellow after 2 weeks. An improvement to the % T 500 nm method for evaluating turbidity/opalescence was established by allowing the samples settle for about 4 hours after appearance testing, then gently mixed to prevent bubble formation, and using a quartz cuvette to read % T @ 500 nm. The steps have allowed for a more robust method for reading % T @ 500 nm. Some of the samples have thickened since week one and were monitored for further gelling.

4-Week Observations: The solutions containing povidone and histidine pH 6.1, sodium phosphate, pH 7.2 or tromethamine pH 8.1 gelled. Some of the solutions became viscous but did not gel. The solutions formulated at low pH or with citrate and polysorbate-80 performed better than povidone. Neither mannitol nor sodium chloride exhibited superior performance over the other.

### Example 7. Pre-Formulations Assessment Using Diglycine Buffer and Excipients

Pre-Formulation studies were performed using a diglycine buffer system with commonly used ophthalmic excipients and commonly used surfactants for stabilizing proteins. Table 12 shows the effect of polyethylene glycol-40 (PEG-40), diglycine buffer and SDP-4 with different sugars (mannitol, trehalose, and sorbitol). Formulations were filtered using a 0.2 μm PES filter to remove particulates and stored in Type I borosilicate glass serum vials under 40° C. temperature conditions and monitored at 3 weeks. All formulations failed the screening process due to the formation of particulates.

TABLE 12
The effect of PEG-40 and diglycine formulation systems

| Ingredient | Form. 1 | Form. 2 | Form. 3 | Form. 4 | Form. 5 |
|---|---|---|---|---|---|
| PEG-40 (% wt./wt.) | 0.5 | 0.5 | 0.5 | 0.5 | 0.5 |
| Diglycine (% wt./wt.) | 0.25 | 0.25 | 0.25 | 0.25 | 0.25 |
| SDP-4 (% wt./wt.) | 1.0 | 1.0 | 1.0 | 1.0 | 1.0 |
| Mannitol (% wt./wt.) | 4.0 | 2.0 | — | 4.0 | 2.0 |
| Trehalose (% wt./wt.) | — | 4.0 | — | — | 4.0 |
| Sorbitol (% wt./wt.) | — | — | 4.0 | — | — |
| pH (% wt./wt.) | 6.4 | 6.9 | 6.9 | 5.6 | 5.5 |
| Osmolarity (mOsm/L) | 254 | 248 | 250 | 248 | 240 |
| Result | Formation | Formation | Formation | Formation of | Formation |
| | of | of | of | Particulates. | of |
| | Particulates. | Particulates. | Particulates. | Failed | Particulates. |
| | Failed | Failed | Failed | Screening | Failed |
| | Screening | Screening | Screening | | Screening |

Additional formulation studies were performed using Tetronic 1107 with diglycine buffer systems. Table 13 shows the effect of Tetronic 1107 with diglycine buffer systems and SDP-4 with glycerol and mannitol. Formulations were filtered using a 0.2 μm PES filter to remove particulates and stored in Type I borosilicate glass serum vials under 40° C. temperature conditions and monitored at 3 weeks. All formulations failed the screening process due to the formation of particulates.

TABLE 13
The effect of Tetronic 1107 and diglycine buffer systems

| Ingredient | Form. 6 | Form. 7 | Form. 8 | Form. 9 |
|---|---|---|---|---|
| Tetronic 1107 | 1.0 | 0.5 | 1.0 | 0.5 |
| (% wt./wt.) | | | | |
| Diglycine | 0.15 | 0.15 | 0.15 | 0.15 |
| (% wt./wt.) | | | | |
| SDP-4 | 1.0 | 1.0 | 1.0 | 1.0 |
| (% wt./wt.) | | | | |
| Glycerol | 2.0 | 2.0 | — | — |
| (% wt./wt.) | | | | |
| Mannitol | — | — | 4.0 | 4.0 |
| (% wt./wt.) | | | | |
| pH | 7.2 | 7.1 | 7.2 | 6.9 |
| Osmolarity | 252 | 254 | 256 | 248 |
| Result | Formation of | Formation of | Formation of | Formation of |
| | Particulates. | Particulates. | Particulates. | Particulates. |
| | Failed | Failed | Failed | Failed |
| | Screening | Screening | Screening | Screening |

### Example 8. Formulation Development Buffer Selection

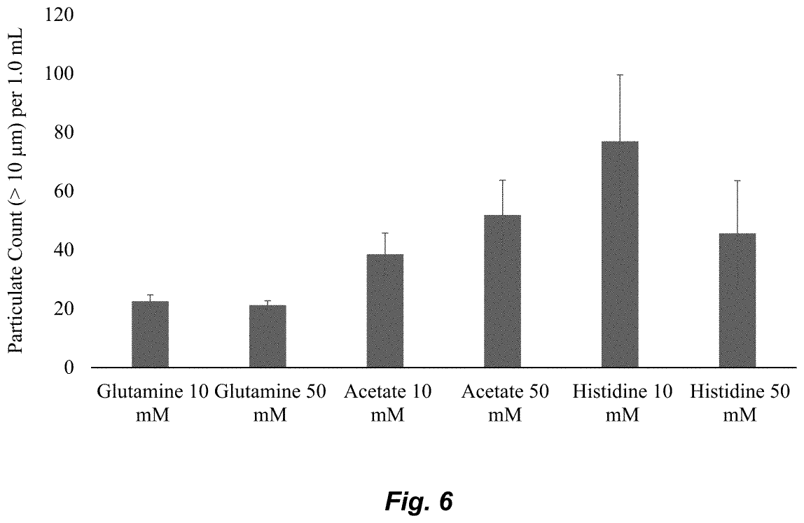

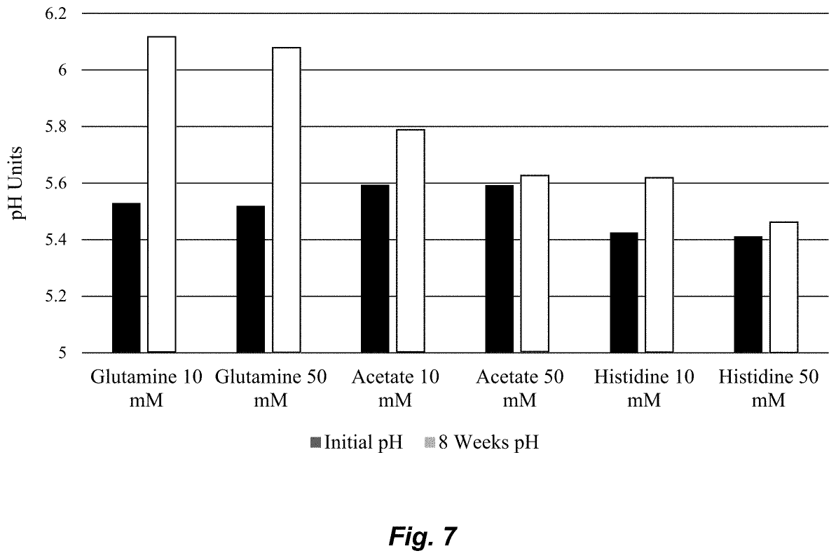

Given the findings in example 1-4, a selection of 3 buffers at a pH of 5.5 were evaluated to achieve the pH requirements of the SDP-4. These buffers include histidine, acetate, and glutamate buffers at concentrations of 10 and 50 mM. Acetate buffers consist of sodium acetate (VWR) and acetic acid (VWR) mixed at specified ratios to reach the desired pH. Histidine (VWR) and glutamine (VWR) buffers were adjusted using 1M Hydrochloric Acid (Lab Chem). Each buffer was adjusted to reach a desired pH value of 5.5. Silk Derived Protein-4 was added to the buffer and diluted with purified water to reach a desired buffer concentration of 10 mM and 50 mM. The final concentration of the SDP-4 in formulation was diluted to 1.0% wt/wt. The formulated SDP-4 was then filtered using polyethersulfone filters (VWR) and aliquoted into Type I, glass borosilicate vials (Prince Sterilization). The vials were placed in a stability chamber at 40° C. for 8 weeks. Initial and final measurements of pH and particulate count were performed using Orion Versa Star pH meter and Coulter Particulate Counter. FIG. 6 represents the particulate count after 8 weeks under storage conditions of 40° C. and 75% relative humidity. Glutamate and acetate buffers inhibited particulate formation relative to the histidine buffer. All glutamate- and acetate-buffered solutions were essentially free of visible particulates. FIG. 7 shows formulation pH initially and after 8 weeks and demonstrates pH drift that occurred in these formulations. Glutamate was insufficient to maintain pH of the SDP-4 while 50 mM acetate and histidine buffers were effective to maintain solution pH over time. Given the effective buffering capacity and the low particulate formation observed, the acetate-buffered formulations were selected for subsequent studies. A final buffer concentration of 25 mM was selected as a midpoint between evaluated buffer strengths, as this would meet requirements to maintain formulation pH.

### Example 9. Effect of Osmolality on SDP-4 Formulations

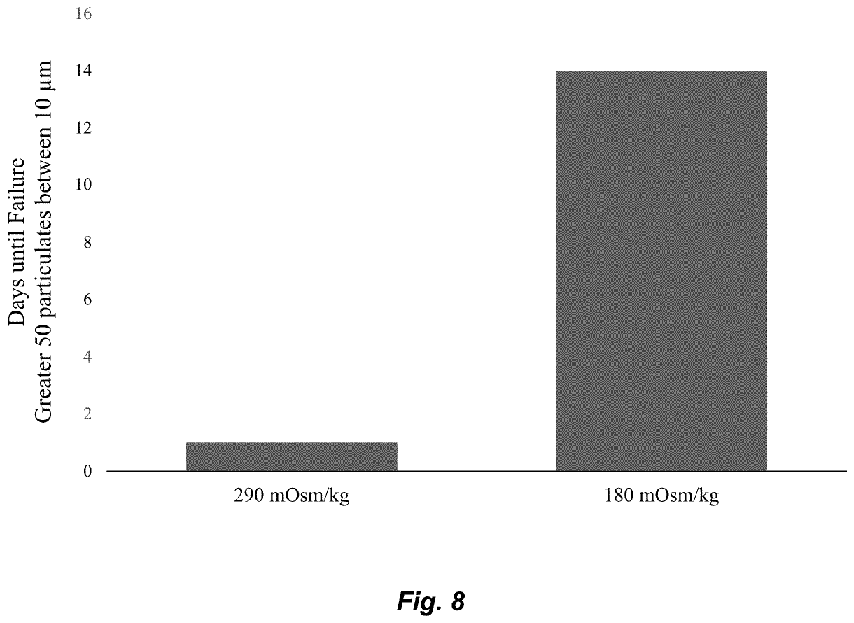

A study was performed to monitor the effect of osmolality on the stability of an acetate buffered formulation. Two formulations, each containing 25 mM acetate buffer and 1.0% wt./wt. SDP-4 were formulated with different levels of mannitol in order to reach an osmolality of 180 and 290 mOsm/kg. The formulated SDP-4 acetate buffered formulations were filtered using PES filters to remove any initial particulates and aliquoted into Type I, glass borosilicate vials. The vials were placed in a stability chamber at 40° C. and monitored daily, FIG. 8 shows the results of these two formulations. It can be seen from the figure that the formulation with an osmolality of 290 mOsm/kg fails within one day where the formulation with an osmolality of 180 mOsm/kg is stable for 14 days. The criteria for a failing formulation is one that exceeds 50 particulates count per mL for particulate sizes between 10 and 25 This study demonstrated that the physical stability of SDP-4 formulations is dependent upon the osmolality of the formulation.

### Example 10. Osmolality Increasing Excipient Selection

The following excipients were considered to increase the osmolality of the formulation: sodium chloride (NaCl), magnesium chloride (MgCl₂), mannitol, and dextrose. Results from an initial screening described in Example 5 excluded commonly used excipients including glycerol, povidone, calcium chloride (CaCl₂)), trehalose, ethylenediaminetetraacetic acid (EDTA), and polyethylene glycol 400 (PEG400), since none of these inhibited particulate formation over time.

Dried Extracted Fiber was reacted on production scale reactor for 240 minutes at the required temperature and pressure. The SDP/LiBr intermediate was fractioned on a benchtop TIT unit resulting in SDP-4. Acetate buffers consisting of sodium acetate (VWR) and acetic acid (VWR) were mixed at a specified ratio to reach the desired acetate buffer pH of 5.4. Excipients were then added to the acetate buffer solution followed by SDP-4. The final concentration of the acetate buffer was 25 mM and the final concentration of SDP-4 was 1.0% wt./′wt. All excipients were added in various amounts to reach the target osmolality of 180 mOsm/kg. The formulations were then filtered using polyethersulfone filters (VWR) and aliquoted into Type I, glass borosilicate vials (Prince Sterilization). The vials were placed in a stability chamber at 25° C. and 40° C. and evaluated for particulates using visual appearance test and Coulter particulate counter. Table 14 shows the list of raw materials used and their manufacturer.

TABLE 14
Excipients and their manufacturer

| | Manu- | | Manu- |
|---|---|---|---|
| Excipient | facturer | Excipient | facturer |
| Sodium Acetate Trihydrate | J.T. Baker | Mannitol | J.T. Baker |
| Glacial Acetic Acid | J.T. Baker | Sodium Chloride | J.T. Baker |
| Super Refined | Croda | Magnesium | J.T. Baker |
| Polysorbate 20 | | Chloride | |
| Super Refined | Croda | Dextrose | J.T. Baker |
| Polysorbate 80 | | | |

Table 15 summarizes 2-week observations of formulations in Type I borosilicate glass. It was identified during visible particulate screening of the MgCl₂ that particulates did not form at 25° C., but started forming shard- and globular-like particulates at 40° C. Dextrose formulations formed fewer particulates than mannitol formulations at 25° C. Additionally, it was observed that salts formed fewer aggregates yet were susceptible to gelation. Conversely, sugars retard gelation yet formed more aggregates. Therefore, a blend of a salt and sugar is optimal to forestall formation of particulates and gelation.

TABLE 15
Excipient Screening for Visible Particulates

| Formulation | | |
|---|---|---|
| (% by | Description of | Description of |
| Osmolality | Particulates | Particulates |
| Contribution) | at 25° C. | at 40° C. |
| 100% | Shard-like particulates, | Shard- and fiber-like particulates |
| Mannitol | <10 per 20 mL | >25 particulates in a 20 mL |
| | of solution. | solution. |
| 100% | Shard-like particulates, | Shard- and globular-like |
| NaCl | <10 per 20 mL | particulates >25 particulates |
| | of solution. | in 20 mL solution. |
| 100% | No visible | Shard- and globular-like |
| MgCh | particulates | particulates >25 particulates |
| | | in a 20 mL solution. |
| 100% | Shard-like particulates, | Shard- and fiber-like particulates |
| Dextrose | <5 per 20 mL | >25 particulates in a |
| | of solution. | 20 mL solution. |
| 50% NaCl/ | Shard-like particulates, | Shard- and globular-like |
| 50% Dextrose | <10 per 20 mL | particulates, between 10 to 25 |
| | of solution. | particulates in a 20 mL solution. |
| 50% MgCh/ | Shard-like particulates, | Shard- and globular-like |
| 50% Dextrose | <5 per 20 mL | particulates, between 10 to 25 |
| | of solution. | particulates in a 20 mL solution. |
| 50% NaCl/ | Shard-like particulates, | Shard- and globular-like |
| 50% Mannitol | <5 per 20 mL | particulates >25 particulates |
| | of solution. | in a 20 mL solution. |
| 50% MgCh/ | Shard-like particulates, | Shard- and globular-like |
| 50% Mannitol | <5 per 20 mL | particulates >25 particulates |
| | of solution. | in a 20 mL solution. |
| 70% NaCl/ | Shard-like particulates, | Shard- and globular-like |
| 30% Mannitol | <10 per 20 mL | particulates >25 particulates |
| | of solution | in a 20 mL solution. |
| 30% NaCl/ | Shard-like particulates, | Shard- and globular-like |
| 70% Mannitol | <10 per 20 mL | particulates >25 particulates |
| | of solution | in a 20 mL solution. |

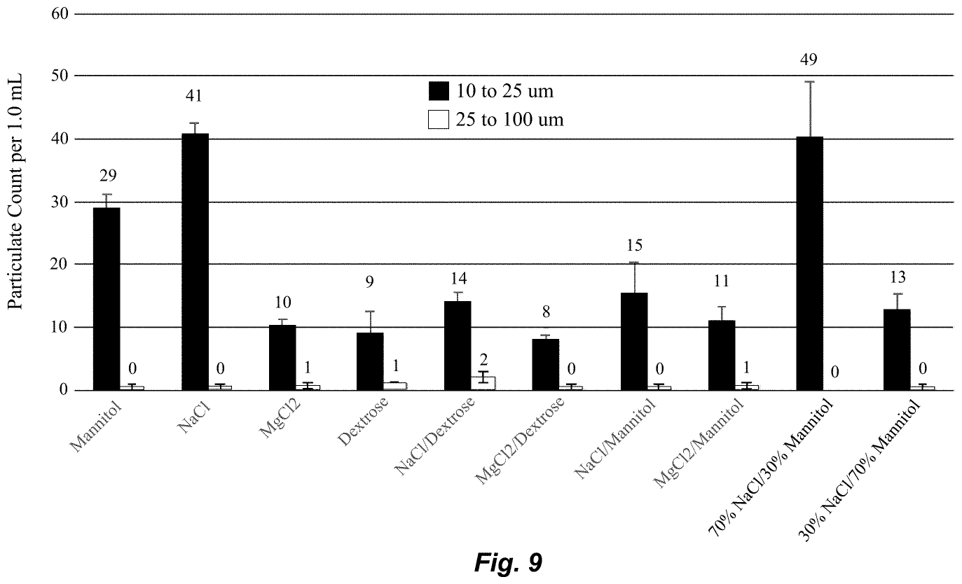

FIG. 9 represents subvisible particulate measurement formulations indicated in Table 15. Magnesium chloride and dextrose form fewer particulate relative to sodium chloride and mannitol. The 50% MgCl₂ and 50% dextrose combination forms the fewest subvisible particulates.

### Example 11. Surfactant Selection

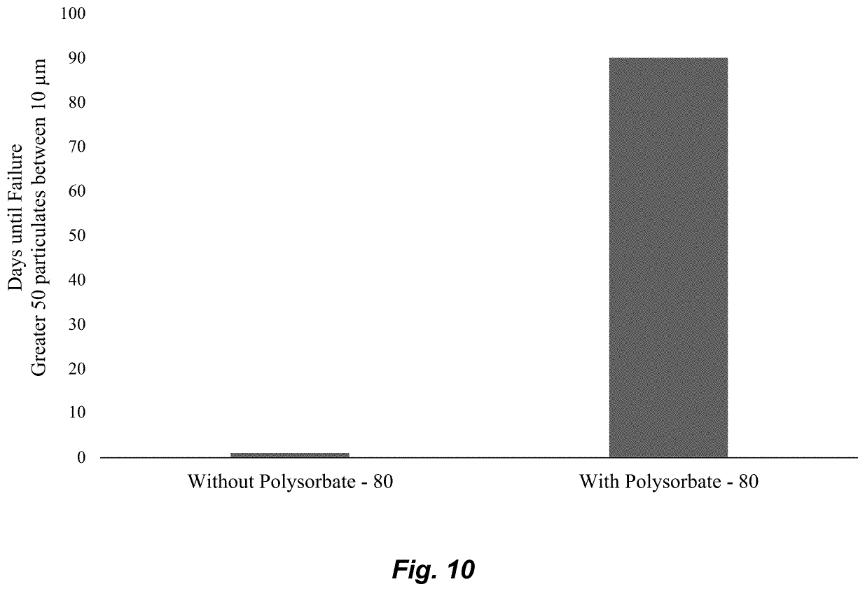

Two compendial surfactants permitted for ophthalmic solutions, poly sorbate-20 and polysorbate-80, were obtained from Croda and evaluated at concentrations indicated in Table 16. All formulations were manufactured using 21.5 mM sodium acetate, 3.5 mM acetic acid, and 1.0% wt./wt, SDP-4 and stored in Type 1 borosilicate glass under 40° C./75% relative humidity for 12 weeks. Analysis of visual appearance, pH, total protein by UV/Vis, and particulate matter tested by the Coulter method were performed. Table 16 identifies the formulation by excipient concentration and Table 17 shows the result of the screening after twelve weeks. Only formulations that passed appearance testing (essentially free of visible particulates) are shown in Table 17; these formulations all contain polysorbate-80. Polysorbate-20 formulations formed high numbers of visual particulates, even more so than formulations that do not contain surfactants. The result of the screening showed that formulations containing polysorbate-80 have passed particulate matter using the criterium in USP <789> and maintained their pH and total protein content. FIG. 10 shows the results of formulations that do not contain polysorbate-80 and FIG. 11 shows the result of particulate count between formulations containing polysorbate-80 and polysorbate-20. The result of this study showed that a surfactant is required to maintain long term physical stability of the SDP-4 formulation. However, the correct surfactant must also be selected. Even though polysorbate-20 and polysorbate-80 are very similar in chemical structure, polysorbate-80 increases stability of SDP-4 formulations by inhibiting particulate formation. Polysorbate-20 accelerates the formation of particulates.

TABLE 16
Surfactant Screening for SDP-4 Formulations

| Formulation | MgCh | Dextrose | Polysorbate-80 | Polysorbate-20 |
|---|---|---|---|---|
| ID | (mM) | (mM) | (% wt./wt.) | (% wt./wt.) |
| 1 | N/A | N/A | N/A | N/A |
| 2 | 38 | 39 | N/A | N/A |
| 3 | 38 | 39 | 0.1% | N/A |
| 4 | 38 | 39 | N/A | 0.1% |
| 5 | 54 | N/A | N/A | N/A |
| 6 | N/A | 130 | N/A | N/A |
| 7 | 38 | 39 | 0.05% | N/A |
| 8 | 38 | 39 | 0.25% | N/A |
| 9 | 38 | 39 | 0.50% | N/A |
| 10 | 54 | N/A | 0.10% | N/A |
| 11 | N/A | 130 | 0.10% | N/A |
| 12 | 38 | 39 | N/A | 0.05% |
| 13 | 38 | 39 | N/A | 0.50% |
| 14 | 54 | N/A | N/A | 0.10% |

TABLE 17
Analysis of Formulation after Twelve Weeks

| | Particulates | | | | Total |
|---|---|---|---|---|---|
| Formulation | Visible | 10 to | >25 | | Protein |
| ID | Particulates | 25 μm | μm | pH | (% wt./wt.) |
| 3 | 1 | 30 | 3 | 5.426 | 1.073 |
| 7 | 1 | 14 | 1 | 5.352 | 1.058 |
| 8 | 0 | 13 | 0 | 5.365 | 1.060 |
| 9 | 0 | 49 | 1 | 5.338 | 1.057 |
| 10 | 0 | 17 | 1 | 5.323 | 1.053 |
| 11 | 1 | 9 | 1 | 5.493 | 1.057 |

### Example 12, Formulation Screening of Standard Ophthalmic Buffers

Additional formulation studies were performed to investigate if other commonly used ophthalmic buffers will produce the same results of inhibiting particulate formation in conjunction with known particulate inhibiting excipients (magnesium chloride, dextrose, polysorbate-80). A selection of three buffers were investigated and includes sodium phosphate, citric phosphate, and tris hydrochloride.

Sodium Phosphate Monobasic, Monohydrate T. Baker) and Sodium Phosphate, Dibasic, 12-Hydrate (J.T. Baker) were mixed in the desired ratio to achieve a pH of 7.0. Citric acid monohydrate and sodium phosphate dibasic were mixed in the desired ratio to achieve a pH of 7.0. Iris hydrochloride (J.T. Baker) was titrated using sodium hydroxide (VWR) to achieve a desired pH of 7.0. Magnesium chloride hexahydrate and dextrose anhydrous were purchased from J.T. Baker and mixed in stock solution. Super Refined Polysorbate 80 was purchased from Croda.

The order of addition for compounding was as follows: polysorbate-80 was initially added, followed by 80% of the water amount, followed by buffer stock solution, followed by magnesium chloride and dextrose. Silk Derived Protein-4 was then added followed by a final addition of water.

The formulation was then filtered using polyethersulfone filters (VWR) and aliquoted into Type I, glass borosilicate vials (Prince Sterilization). The vials were placed in a stability chamber at 40° C. and 75% Relative Humidity and evaluated for particulates using visual appearance testing. The results of the screening can be seen in Table 18,

TABLE 18
Formulation Screening of Standard Ophthalmic Buffers

| | | Osmo- | | |
|---|---|---|---|---|
| | pH of | lality | Time in | |
| | formu- | (mOsm/ | Stability | |
| Formulation | lation | kg) | Chamber * | Result |
| 20 mM Sodium | 7.1 | 167 | 0 Days | Formation of |
| Phosphate, 38 mM | | | | insoluble |
| Magnesium Chloride, | | | | particulates |
| 39 mM Dextrose, | | | | during |
| 0.05% | | | | titration with |
| wt./wt. Polysorbate- | | | | Sodium |
| 80, and 1.0% wt./wt. | | | | Hydroxide. |
| SDP-4 | | | | Failed |
| | | | | screening. |
| 20 mM Citric | 7.0 | 190 | 0 Days | Formation of |
| Phosphate, 29 Mm | | | | insoluble |
| Magnesium Chloride, | | | | particulates |
| 29 mM Dextrose, | | | | during |
| 0.05% wt./wt. | | | | titration with |
| Polysorbate- | | | | Sodium |
| 80, and 1.0% wt./wt. | | | | Hydroxide. |
| SDP-4 | | | | Failed |
| | | | | screening. |
| 20 mM Tris | 6.9 | 190 | 6 Weeks | Greater than |
| Hydrochloride, 40 | | | | 2 visible |
| mM Magnesium | | | | particulate |
| Chloride, 41 mM | | | | per mL. |
| Dextrose, 0.05% | | | | Failed |
| wt./wt, Polysorbate- | | | | screening. |
| 80, and 1.0% wt./wt. | | | | |
| SDP-4 | | | | |

* Time in Stability Chamber = 40° C./75% Relative Humidity.

The results of the study show that formulations compounded using sodium phosphate, citric phosphate, and tris hydrochloride in conjunction with known particulate inhibiting excipients (magnesium chloride, dextrose, polysorbate-80) does not inhibit particulate formation. Two of the buffers, sodium phosphate and citric phosphate, immediately formed insoluble particulates during titration to the desired pH. Tris hydrochloride failed the visual appearance screening test after 6 weeks under 40° C., storage conditions.

### Example 13, Formulation Development Using Standard Surfactants

Additional formulation studies were performed to investigate if other commonly used ophthalmic surfactants will produce the same results of inhibiting particulate formation in conjunction with known particulate inhibiting excipients (magnesium chloride, dextrose, acetate). A selection of four surfactants were investigated and includes poloxamer 188, poloxamer 407, polyethylene glycol 300, polyethylene glycol 400, and polyethylene glycol 600.

The order of addition for compounding was as follows: 80% of the desired water amount was added, followed by direct addition of surfactants, magnesium chloride, dextrose, sodium acetate trihydrate and glacial acetic acid. The formulation was then mixed until all excipients were full dissolved, Silk Derived Protein-4 was then added, followed by a final addition of water.

The formulation was then filtered using polyethersulfone filters (VWR) and aliquoted into Type I, glass borosilicate vials (Prince Sterilization). The vials were placed in a stability chamber at 40° C. and 75% Relative Humidity and evaluated for particulates using visual appearance testing. The results of the screening can be seen in Table 19.

TABLE 19
Formulation screening using standard ophthalmic surfactants.

| | | Osmo- | | |
|---|---|---|---|---|
| | pH of | lality | Time in | |
| | formu- | (mOsm/ | Stability | |
| Surfactant | lation | kg) | Chamber * | Result |
| 20 mM acetate buffer, 37 | 5.5 | 180 | 1 Week | Greater than 2 |
| mM magnesium chloride, | | | | visible |
| 38 mM dextrose, 1.0% | | | | particulate |
| wt./wt. SDP-4, and | | | | per mL |
| Poloxamer 188 (0.05% | | | | Failed |
| wt./wt.) | | | | screening. |
| 20 mM acetate buffer, 37 | 5.5 | 179 | 3 Weeks | Greater than 2 |
| mM magnesium chloride, | | | | visible |
| 38 mM dextrose, 1.0% | | | | particulate |
| wt./wt. SDP-4, and | | | | per mL |
| Poloxamer 407 | | | | Failed |
| (0.05% wt./wt.) | | | | screening. |
| 20 mM acetate buffer, 37 | 5.5 | 181 | 1 Week | Greater than 2 |
| mM magnesium chloride, | | | | visible |
| 38 mM dextrose, 1.0% | | | | particulate |
| wt./wt. SDP-4, and | | | | per mL |
| Polyethylene Glycol | | | | Failed |
| (PEG) 300 | | | | screening. |
| (0.05% wt./wt.) | | | | |
| 20 mM acetate buffer, 37 | 5.5 | 180 | 2 Weeks | Greater than 2 |
| mM magnesium chloride, | | | | visible |
| 38 mM dextrose, 1.0% | | | | particulate |
| wt./wt. SDP-4, and | | | | per mL |
| Polyethylene Glycol | | | | Failed |
| (PEG) 400 | | | | screening. |
| (0.05% wt./wt.) | | | | |
| 20 mM acetate buffer, 37 | 5.5 | 182 | 2 Weeks | Greater than 2 |
| mM magnesium chloride, | | | | visible |
| 38 mM dextrose, 1.0% | | | | particulate |
| wt./wt. SDP-4, and | | | | per mL |
| Polyethylene Glycol | | | | Failed |
| (PEG) 600 | | | | screening. |
| (0.05% wt./wt.) | | | | |

* Time in Stability Chamber = 40° C./75% Relative Humidity.

The results of the study show that formulations compounded using poloxamer 188, poloxamer 407, PEG-300, PEG-400, and PEG-600 with other particulate inhibiting excipients (magnesium chloride, dextrose, polysorbate-80) do not inhibit particulate formation. For all items in Table 19, the surfactants failed the screening process between 1 and 3 weeks.

### Example 14. SDP-4 Ophthalmic Solution Drug Product

Dosage Form. Silk-Derived Protein-4 (SDP-4) Sterile Topical Ophthalmic Solution Drug Product (DP) contains SDP-4 Drug Substance (DS) in single-use vials. The osmotic agents are adjusted to establish an osmolality of 180 mOsm/kg (±1%) (Tables 21-23).

TABLE 21
Composition of SDP-4 0.1% wt./wt. Drug Product Formulation

| | Amount per | | |
|---|---|---|---|
| Component | unit (wt./wt.) | Function | Quality Standard |
| SDP-4 DS | 0.1% | Drug Substance | See Specification |
| Sodium Acetate | 0.248% | Buffering Agent | USP |
| Trihydrate | | | |
| Glacial Acetic Acid | 0.011% | Buffering Agent | USP |
| Magnesium Chloride | 0.813% | Osmotic agent | USP |
| Hexahydrate | | | |
| Dextrose | 0.813% | Osmotic agent | USP |
| Monohydrate | | | |
| Polysorbate-80 | 0.050% | Surfactant | USP, Super Refined |

TABLE 22
Composition of SDP-4 1.0% wt./wt. Drug Product Formulation

| | Amount per | | |
|---|---|---|---|
| Component | unit (wt./wt.) | Function | Quality Standard |
| SDP-4 DS | 1.0% | Drug Substance | See Specification |
| Sodium Acetate | 0.248% | Buffering Agent | USP |
| Trihydrate | | | |
| Glacial Acetic Acid | 0.011% | Buffering Agent | USP |
| Magnesium Chloride | 0.752% | Osmotic agent | USP |
| Hexahydrate | | | |
| Dextrose | 0.753% | Osmotic agent | USP |
| Monohydrate | | | |
| Polysorbate-80 | 0.050% | Surfactant | USP, Super Refined |

TABLE 23
Composition of SDP-4 3.0% wt./wt. Drug Product Formulation

| | Amount per | | |
|---|---|---|---|
| Component | unit (wt./wt.) | Function | Quality Standard |
| SDP-4 DS | 3.0% | Drug Substance | See Specification |
| Sodium Acetate | 0.248% | Buffering Agent | USP |
| Trihydrate | | | |
| Glacial Acetic Acid | 0.011% | Buffering Agent | USP |
| Magnesium Chloride | 0.651% | Osmotic agent | USP |
| Hexahydrate | | | |
| Dextrose | 0.654% | Osmotic agent | USP |
| Monohydrate | | | |
| Polysorbate-80 | 0.050% | Surfactant | USP, Super Refined |

Type of Container and Closure for Dosage Form. The DP was supplied in single unit dose (SUD) low-density polyethylene (LDPE) vial with a 0.512-0.589 g fill range. The DP and the vial underwent blow-fill-seal (BFS) manufacturing utilizing a sterile filling process of DP into the BFS vial allowing for 20 μL-50 μL drop volume size.

Type of Container and Closure for Drug Product. A sealed SUD with a 1 mL total liquid volume capacity was produced from LDPE using a Blow-Fill-Seal (BFS) process.

TABLE 24
Composition of SDP-4 Drug Product Formulation

| | Amount per BFS | | Quality |
|---|---|---|---|
| Component | unit (wt./wt.) | Function | Standard |
| SDP-4 DS | 0.1 to 15.0% wt./wt. | Drug Substance | See |
| | | | specification |
| Sodium Acetate | 0.10 to 1.0% wt./wt. | Buffering Agent | USP |
| Trihydrate | | | |
| Glacial Acetic Acid | 0.01 to 0.1% wt./wt. | Buffering Agent | USP |
| Magnesium Chloride | 0.10 to 2.0% wt./wt. | Osmotic agent | USP |
| Hexahydrate | | | |
| Dextrose | 0.10 to 2.0% wt./wt. | Osmotic agent | USP |
| Monohydrate | | | |
| Polysorbate - 80 | 0.02 to 2.0% wt./wt. | Surfactant | USP, Super |
| | | | Refined |

Stability studies were performed on the formulations contained in Tables 21-23. The environmental conditions of the stability studies were 40° C./75% relative humidity. Initial measurements were taken at the time of manufacture and at the 6-month time point. Each formulation was tested for visual appearance, pH, osmolality, and particulate matter. Tables 25-27 shows the results of the stability studies. The formulations have been shown to be inhibit particulate formation, maintain solution pH and osmolality under conditions of 40° C./75% relative humidity in a low-density polyethylene container closure. The development and summation of the formulation work resulted in a formulation that meets all specification in a container closure that is favorable to commercial ophthalmic under storage conditions that are normally unfavorable to therapeutic proteins.

TABLE 25
Stability results of SDP-4 0.1% wt./wt. Drug Product Formulation

| Attribute | Specification | Initial | 6 Month |
|---|---|---|---|
| Appearance | Clear, slightly | Clear, slightly | Clear, slightly |
| | yellow, | yellow, | yellow, essentially |
| | essentially free of | essentially free of | free of visible |
| | visible particulates | visible particulates | particulates |
| pH | 5.2-5.7 | 5.5 | 5.6 |
| Osmolality | 160-200 mOsm/kg | 187 | 189 |
| Particulate | Particles ≥10 μm | Particles ≥10 μm | Particles ≥10 μm |
| Matter | No more than | 2 per mL | 4 per mL2 |
| | (NMT) 50 per mL | Particles ≥25 μm | Particles ≥25 μm |
| | Particles ≥25 μm | 0 per mL | 1 per mL2 |
| | NMT 5 per mL | Particles ≥50 μm | Particles ≥50 μm |
| | Particles ≥50 μm | 0 per mL | 1 per mL2 |
| | NMT 2 per mL | | |
| Meets | PASS | PASS | PASS |
| Specification | | | |

TABLE 26
Stability results of SDP-4 1.0% wt./wt. Drug Product Formulation

| Attribute | Specification | Initial | 6 Month |
|---|---|---|---|
| Appearance | Clear, slightly | Clear, slightly | Clear, slightly |
| | yellow, essentially | yellow, essentially | yellow, essentially |
| | free of visible | free of visible | free of visible |
| | particulates | particulates | particulates |
| pH | 5.2-5.7 | 5.5 | 5.6 |
| Osmolality | 160-200 mOsm/kg | 183 | 187 |
| Particulate | Particles ≥10 μm | Particles ≥10 μm | Particles ≥10 μm |
| Matter | NMT 50 per mL | 2 per mL | 6 per mL2 |
| | Particles ≥25 μm | Particles ≥25 μm | Particles ≥25 μm |
| | NMT 5 per mL | 0 per mL | 3 per mL2 |
| | Particles ≥50 μm | Particles ≥50 μm | Particles ≥50 μm |
| | NMT 2 per mL | 0 per mL | 0 per mL2 |
| Meets | PASS | PASS | PASS |
| Specification | | | |

TABLE 27
Stability results of SDP-4 3.0% wt./wt. Drug Product Formulation

| Attribute | Specification | Initial | 6 Month |
|---|---|---|---|
| Appearance | Clear, slightly | Clear, slightly | Clear, slightly |
| | yellow,e ssentially | yellow, essentially | yellow, essentially |
| | free of visible | free of visible | free of visible |
| | particulates | particulates | particulates |
| pH | 5.2-5.7 | 5.5 | 5.6 |
| Osmolality | 160-200 mOsm/kg | 162 | 182 |
| Particulate | Particles ≥10 μm | Particles ≥10 μm | Particles ≥10 μm |
| Matter | NMT 50 per mL | 5 per mL | 11 per mL2 |
| | Particles ≥25 μm | Particles ≥25 μm | Particles ≥25 μm |
| | NMT 5 per mL | 0 per mL | 3 per mL2 |
| | Particles ≥50 μm | Particles ≥50 μm | Particles ≥50 μm |
| | NMT 2 per mL | 0 per mL | 2 per mL2 |
| Meets | PASS | PASS | PASS |
| Specification | | | |

### Example 15. Treatment of Dry Eye: Ophthalmic Formulations with and without SDP-4

The primary objective of this study was to assess the safety and efficacy of SDP-4 Ophthalmic Solution in subjects with DED over a 12-week (84-day) treatment period.

Study Design:

This was a Phase 2, multicenter, double-masked, randomized, vehicle-controlled, dose-response, parallel-group study designed to evaluate the ocular and systemic safety and efficacy of SDP-4 ophthalmic solution in subjects with moderate to severe DED in both eyes (OU) over a 12-week (84-day) treatment period.

Subjects were randomized to 1 of 3 concentrations (0.1%, 1.0% and 3.0%) of SDP-4 Ophthalmic Solution or vehicle in a 1:1:1:1 ratio in parallel groups. All investigational products (IP) (SDP-4 concentrations and vehicle) were provided in single-use dose (SUD) containers seal packed into foil pouches. Subjects, the Investigator, and all site personnel responsible for performing study assessments remained masked to treatment assignment.

The IP was administered via topical ocular instillation, one drop per eye, twice daily (BID) for 12 weeks (84 days). Both eyes were treated. A 2-week screening/run-in period on BID vehicle preceding the 12-week randomized treatment period.

Subjects must have had a Symptom Assessment in Dry Eye (SANDE) total score of ≥40 at Visit 1/Screening and Visit 2/Day 1 to enter the trial. For subjects with a qualifying SANDE score who meet all other inclusion/exclusion criteria, the eye with the lower tear break-up time (TBUT) at Visit 2/Day 1 was designated as the study eye. In the event both eyes have the same TBUT scores, the eye with the lower Schirmer's test score was designated as the study eye. If both eyes have the same TBUT and Schirmer's test scores, the right eye was designated as the study eye.

The study consisted of 7 clinic visits, 2 visits during the screening period and 5 on treatment visits: Visit 1 (Day-14±2/Screening Visit), followed by the 2-week run-in period on BID vehicle, Visit 2 (Day 1/Confirmatory and Randomization Visit), Visit 3 (Day 7±2), Visit 4 (Day 14±2), Visit 5 (Day 28±2), Visit 6 (Day 56±4) and Visit 7 (Day 84±4/End of Study Assessments).

If a subject complained of persistent dry eye symptoms, the site was allowed to provide the subject with unpreserved artificial tears (provided by the Sponsor), to be used only if necessary. The subject was to return all used and unused artificial tears at each visit so the site can conduct accountability to assess the use of artificial tears. Artificial tears could not be used within 2 hours prior to any study visit.

### Efficacy Measurements

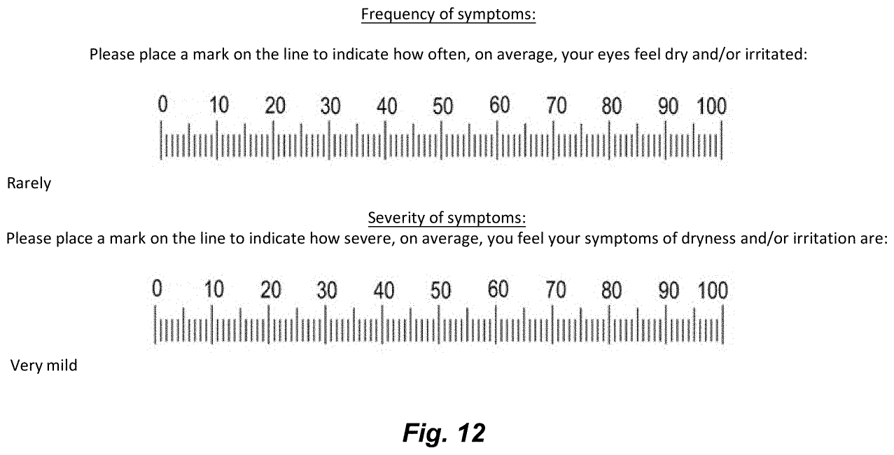

Efficacy was measured by assessment of DED symptoms (SANDE total score, individual symptoms rated on a visual analogue scale (VAS): itching, foreign body sensation, burning/stinging, fluctuating vision, eye dryness, eye discomfort, photophobia, and eye pain) and signs (TBUT, Schirmer's test [anesthetized], corneal fluorescein staining, conjunctival lissamine green staining, and conjunctival hyperemia) (FIG. 12). All efficacy assessments were conducted at the timepoints shown on the Schedule of Visits and Examinations.

### Primary Efficacy Variable

The primary efficacy endpoint (SANDE) was summarized using continuous summary statistics by treatment group and visit. The primary analysis utilized a repeated measures mixed model where the dependent variable is the change from baseline score, treatment group is a fixed effect, baseline score is a covariate, and visit is a repeated measure on subject. The repeated measures mixed model was utilized to account for the effect of missing data under the assumption that the data are missing at random. Least squares means were used to test each concentration of SDP-4 to vehicle. Sensitivity analyses for the primary endpoint was performed using last observation carried forward (LOCF).

### Analysis of Efficacy

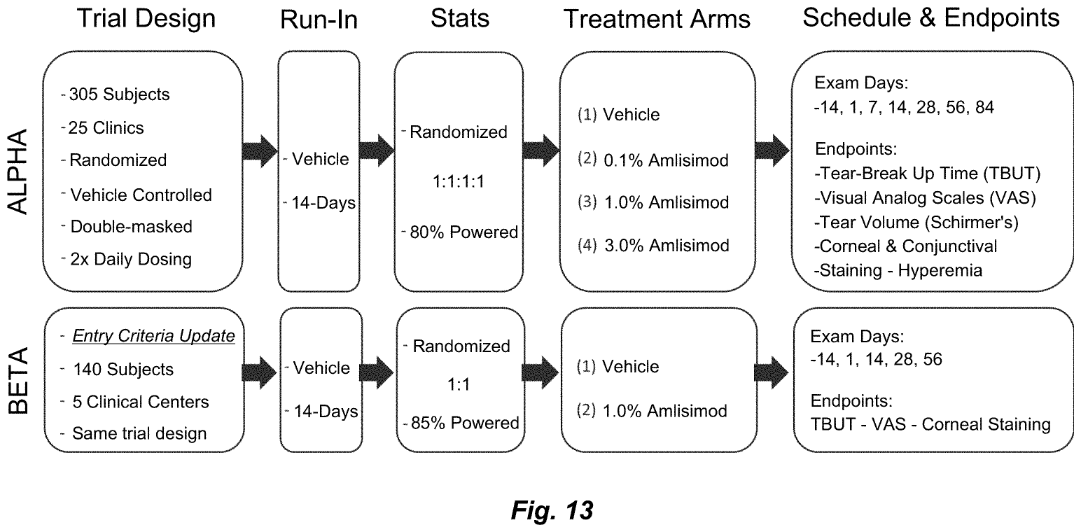

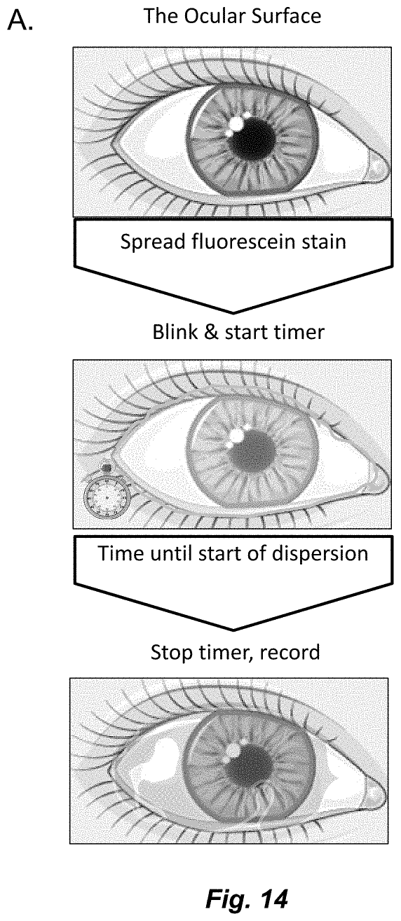

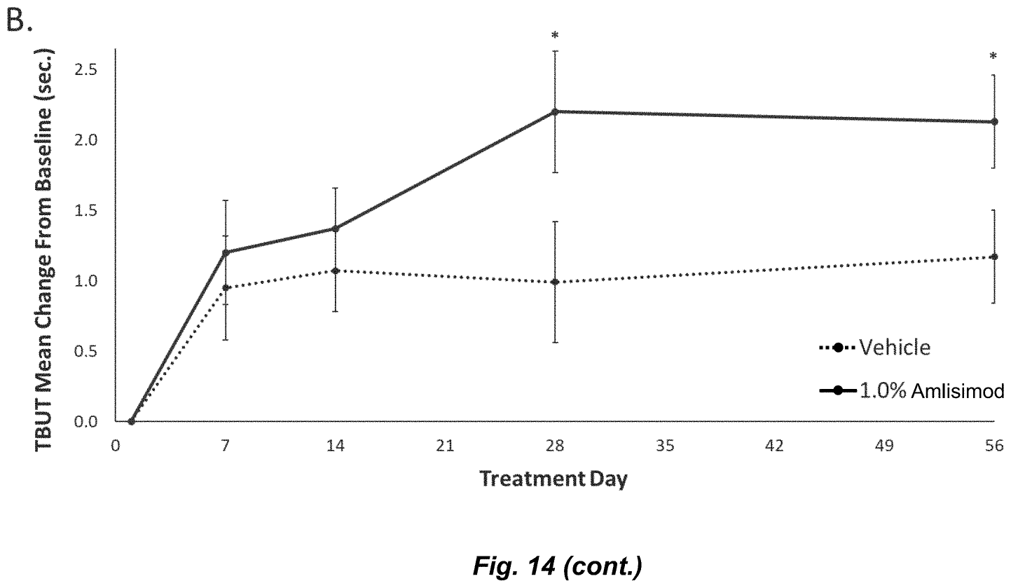

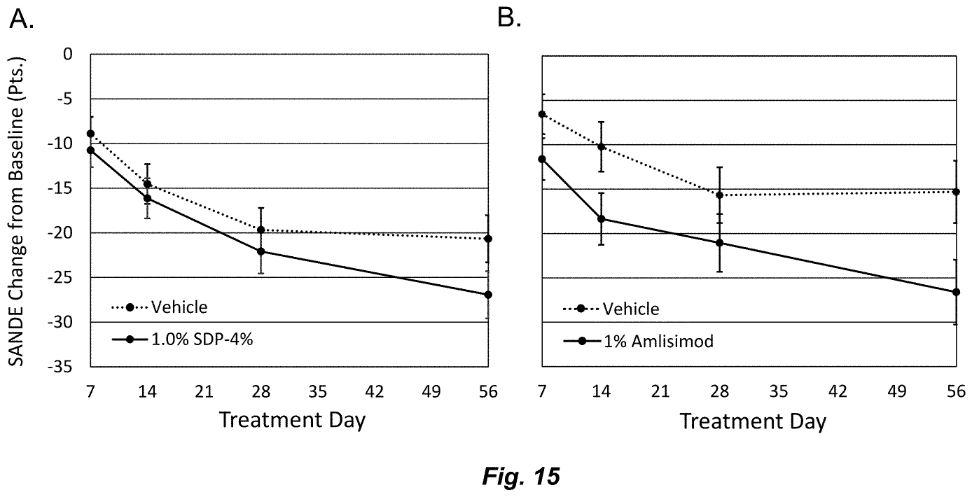

At baseline, mean total SANDE score ranged from 67 to 71 units (0-100 scale). This measure improved (decreasing value) starting at Day 7 in all treatment groups and continuing to improve throughout the study. At Day 84, the primary outcome measure, mean reduction in this measure was 25, 30, 25 and 26 in the 0.1%, 1.0% and 3.0% SDP-4 and vehicle groups, respectively. See Table 28, FIGS. 13-15. The mean total SANDE score difference (active group versus vehicle group) for a patient population that included patients with starting SANDE scores greater than or equal to 70 was not statistically significant until after 28 days (p=0.2839 to 0.7952, FIG. 15A). Therefore the vehicle formulation also provides an effective treatment for the symptoms of dry eye. However, the Amlisimod 1% formulation was statistically more effective than the vehicle for patient subpopulations that did not include patients with starting SANDE scores greater than or equal to 70 and showed a significant improvement over the whole population (FIG. 15B), demonstrating that the SDP-4 (Amlisimod) formulation (Table 22) is highly effective for the treatment of dry eye.

TABLE 28
Mean Change from Baseline in Total SANDE Score (ITT Population)

| Visit | Vehicle |
|---|---|
| Statistics | (N = 76) |
| Screening | |
| N | 76 |
| Mean (SD) | 73.00 (13.949) |
| Median | 74.83 |
| Min, Max | 44.7, 100.0 |
| Day 1 | |
| N | 76 |
| Mean (SD) | 69.22 (15.087) |
| Median | 66.67 |
| Min, Max | 42.4, 100.0 |
| Day 7 | |
| N | 76 |
| Mean (SD) | 60.30 (20.663) |
| Median | 64.91 |
| Min, Max | 0.0, 100.0 |
| Change from Baseline | |
| n | 76 |
| Mean (SD) | −8.92 (15.906) |
| Median | −4.92 |
| Min, Max | −60.5, 14.8 |
| LS Mean, SE (1) | −8.90, 1.88 |
| LSM Difference, SE (2) | |
| 95% CIs (2) | |
| p value (3) | |
| Day 14 | |
| n | 76 |
| Mean (SD) | 54.67 (24.179) |
| Median | 57.28 |
| Min, Max | 5.7, 99.5 |
| Change from Baseline | |
| n | 76 |
| Mean (SD) | −14.55 (21.156) |
| Median | −8.35 |
| Min, Max | −76.0, 26.3 |
| LS Mean, SE (1) | −14.53, 2.23 |
| LSM Difference, SE (2) | |
| 95% CIs (2) | |
| p value (3) | |
| Day 28 | |
| n | 76 |
| Mean (SD) | 49.54 (26.147) |
| Median | 49.75 |
| Min, Max | 2.7, 99.5 |
| Change from Baseline | |
| n | 76 |
| Mean (SD) | −19.68 (22.805) |
| Median | −12.06 |
| Min, Max | −77.8, 10.2 |
| LS Mean, SE (1) | −19.66, 2.45 |
| LSM Difference, SE (2) | |
| 95% CIs (2) | |
| p value (3) | |
| Day 56 | |
| n | 76 |
| Mean (SD) | 48.52 (26.605) |
| Median | 50.00 |
| Min, Max | 2.0, 100.0 |
| Change from Baseline | |
| n | 76 |
| Mean (SD) | −20.70 (25.790) |
| Median | −13.56 |
| Min, Max | −90.0, 22.4 |
| LS Mean, SE (1) | −20.67, 2.64 |
| LSM Difference, SE (2) | |
| 95% CIs (2) | |
| p value (3) | |
| Day 84 | |
| n | 76 |
| Mean (SD) | 43.56(27.691) |
| Median | 45.45 |
| Min, Max | 0.0, 100.0 |
| Change from Baseline | |
| n | 76 |
| Mean (SD) | −25.66 (26.685) |
| Median | −18.75 |
| Min, Max | −85.9, 26.3 |
| LS Mean, SE (1) | −25.64, 2.89 |
| LSM Difference, SE (2) | |
| 95% CIs (2) | |
| p value (3) | |

Scale: 0-100 (none to severe)
Test statistics and estimates are from a restricted maximum likelihood repeated measures mixed model on change from baseline values with baseline as a covariate
and visit, and its interaction with treatment group as repeated measures using an unstructured covariance structure.
(1) Least square (LS) mean and standard error (SE) per treatment group.
(2) Treatment Effect: Least square mean (LSM) difference, standard error (SE), and 95% confidence intervals (CIs) between SDP-4 and vehicle.
(3) p value comparing SDP-4 and vehicle.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A formulation comprising:
(a) a fibroin-derived protein composition wherein the primary amino acid sequences of the fibroin-derived protein composition differ from native fibroin by at least 4% with respect to the absolute values of the combined differences in amino acid content of serine, glycine, and alanine;
cysteine disulfide bonds between the fibroin heavy and fibroin light protein chains of the fibroin-derived protein are reduced or eliminated; the protein composition has a serine content that is reduced by greater than 25% compared to native fibroin, wherein the serine content is at least about 5%; and the average molecular weight of the fibroin-derived protein composition is between 15 kDa and 36 kDa;
(b) polysorbate-80;
(c) one or more buffering agents, wherein the buffering agent comprises histidine, acetate, citrate, glutamate, or a combination thereof, wherein a buffer concentration formed by the buffering agent is about 10 millimolar to about 50 millimolar, or the concentration of each of the one or more osmotic agents in the formulation is about 30 millimolar to about 40 millimolar, and wherein the buffering agent comprises about 0.1 wt. % to about 1 wt. % sodium acetate and about 0.01 wt. % to about 0.1 wt. % acetic acid;
(d) one or more osmotic agents; and
wherein the formulation has a pH of 4.5 to 6.0 and a particulate count of 50/mL or less after a storage period of greater than 12 weeks at 4° C. to 40° C., with respect to particulates having a diameter of 10 micrometers or more.

2. The formulation of claim 1 wherein the protein composition comprises greater than 46.5% glycine amino acids, the protein composition comprises greater than 30.5% alanine amino acids, or a combination thereof.

3. The formulation of claim 1 wherein the protein composition has a serine content that is reduced by greater than 40% compared to native fibroin protein such that the protein composition comprises less than 8% serine amino acids.

4. The formulation of claim 1 wherein greater than 50% of the protein chains of the protein composition have a molecular weight within the range of 10 kDa to 40 kDa.

5. The formulation of claim 1 wherein the primary amino acid sequences of the fibroin-derived protein composition differ from native fibroin by at least by at least 6% with respect to the combined difference in serine, glycine, and alanine content; the average molecular weight of the fibroin-derived protein is about 15 kDa to about 30 kDa; and the pH of the formulation is between about 5.2 and about 5.8.

6. The formulation of claim 1 wherein the fibroin-derived protein composition has an average molecular weight of about 15 kDa to about 25 kDa, and the pH of the formulation is 5.3 to 5.7.

7. The formulation of claim 1 wherein the fibroin-derived protein composition has an average molecular weight of about 18 kDa to about 25 kDa.

8. The formulation of claim 1 wherein the wt. % of the fibroin-derived protein is about 0.05% to about 10%.

9. The formulation of claim 1 wherein the osmolality of the formulation is about 170 mOsm/kg to about 300 mOsm/kg.

10. (canceled)

11. (canceled)

12. (canceled)

13. (canceled)

14. (canceled)

15. The formulation of claim 1 wherein the osmotic reagent comprises mannitol, dextrose, sodium chloride, magnesium chloride, or a combination thereof; wherein the osmotic reagent comprises about 0.10 wt. % to about 2 wt. % dextrose and about 0.10 wt. % to about 2 wt. % magnesium chloride.

16. The formulation of claim 1 wherein the wt. % of polysorbate-80 is about 0.02% to about 2%.

17. The formulation of claim 1 wherein the formulation is stored in a vessel comprising glass or polyethylene, wherein optionally, the vessel is Type I borosilicate glass or LDPE.

18. (canceled)

19. An aqueous formulation comprising:
(a) about 0.1 wt. % to about 3 wt. % fibroin-derived protein wherein the primary amino acid sequences of the fibroin-derived protein differ from native fibroin by at least 6% with respect to the absolute values of the combined differences in amino acid content of serine, glycine, and alanine; cysteine disulfide bonds between the fibroin heavy and fibroin light protein chains of the fibroin-derived protein are reduced or eliminated; the fibroin-derived protein comprises greater than 46% glycine amino acids and greater than 30% alanine amino acids; the fibroin-derived protein has a serine content that is reduced by greater than 40% compared to native fibroin protein such that the fibroin-derived protein comprises less than 8% serine amino acids; and the average molecular weight of fibroin-derived protein is about 15 kDa to about 35 kDa;
(b) polysorbate-80;
(c) about 10 millimolar to about 50 millimolar acetate buffer, wherein the acetate buffer comprises about 0.2 wt. % to about 0.3 wt. % sodium acetate and about 0.01 wt. % to about 0.03 wt. % acetic acid; and
(d) one or more osmotic agents;
wherein the formulation has a pH of 5.2 to 5.8; an osmolality of 175 mOsm/kg to 185 mOsm/kg; and a particulate count of 50/mL or less after a storage period of greater than 12 weeks at 4° C. to 40° C., with respect to particulates having a diameter of 10 micrometers or more.

20. The formulation of claim 19 wherein the osmotic agent comprises about 0.6 wt. % to about 0.9 wt. % dextrose and about 0.6 wt. % to about 0.9 wt. % magnesium chloride hexahydrate.

21. The formulation of claim 20 wherein the wt. % of polysorbate-80 is about 0.01% to about 0.1%.

22. A method for treating an ophthalmic disease comprising administering an effective amount of the formulation of claim 1 to a subject having an ophthalmic disease, thereby treating the ophthalmic disease.

23. The method of claim 22 wherein the ophthalmic disease is Dry Eye Syndrome.


SEQUENCE LISTING (1 sequence)

SEQ ID NO: 1 - protein, 4 aa, Artificial Sequence
  Description of Artificial Sequence Synthetic peptide
GAGA